(19) United States
(12) Patent Application Publication
(10) Pub. No.: US 2017/0313735 A1
(43) Pub. Date: Nov. 2, 2017

(54) Improved Fluorination Process

(71) Applicant: Sandoz AG, Basel (CH)

(72) Inventors: Olga Schone, Kundl (AT); Thorsten Wilhelm, Kundl (AT); Hans-Peter Spitzenstatter, Kundl (AT); Julia Juen, Kundl (AT)

(73) Assignee: Sandoz AG, Basel (CH)

(21) Appl. No.: 15/522,199

(22) PCT Filed: Jun. 22, 2015
(86) PCT No.: PCT/EP2015/063999

§ 371 Date: Apr. 26, 2017

(30) Foreign Application Priority Data
Oct. 31, 2014 (EP) 14191279.0

(51) Int. Cl. C07H 19/06

(52) U.S. Cl. CPC C07H 19/06

(57) ABSTRACT

A process comprising (i) providing a mixture comprising a compound of formula (I) or isomers, stereoisomers, diastereomers, enantiomers or salts thereof; (ii) subjecting the mixture provided in (i) to fluorinating conditions in the presence of a fluorination agent selected from the group consisting of diethylamino (difluoro) sulfonium tetrafluoroborate and difluoro(morpholino) sulfonium tetrafluoroborate obtaining a mixture comprising a compound of formula (II) or isomers, stereoisomers diastereomers, enantiomers or salts thereof; (iii) optionally subjecting the mixture obtained in (ii) to deprotection conditions, obtaining a mixture comprising the compound of formula (III) or isomers, stereoisomers, diastereomers, enantiomers or salts thereof.

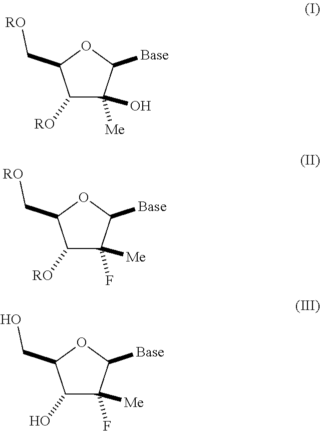

The present invention relates to a novel process for the synthesis of 2′-deoxy-2′-fluoro-2′-C-methyl-ribofuranosyl nucleosides as well as to novel intermediate compounds. The present invention further relates to the use of said intermediates for the preparation of nucleoside phosphoramidate derivatives such as sofosbuvir.

Sofosbuvir according to formula (A)

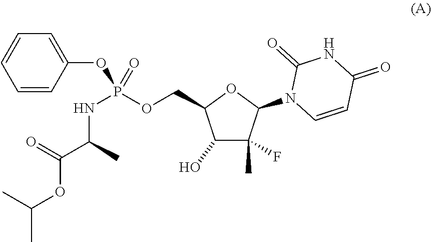 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate is a drug inhibiting the RNA polymerase used by the hepatitis C virus to replicate its RNA. 2′-deoxy-2-fluoro-2′C-methyluridine of formula

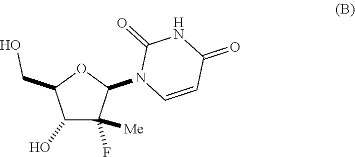 [SMILES: C[C@@]1(F)[C@H](O)[C@@H](CO)O[C@H]1n1ccc(=O)[nH]c1=O]

is a 2′-deoxy-2′-fluoro-2′-C-methyl-ribofuranosyl nucleoside and an intermediate in the synthesis of sofosbuvir.

There are two general approaches for the synthesis of 2′-deoxy-2-fluoro-2′C-methyluridine or, in general, for the synthesis of 2′-deoxy-2′-fluoro-2′-C-methyl-ribofuranosyl nucleosides.

The first approach is the de novo synthesis of a fluorinated sugar (ribonolactone or ribofuranosyl) using early-stage fluorination or a simple fluorinated building block. The sugar is then coupled with the nucleobase to make the nucleoside, according to the following scheme:

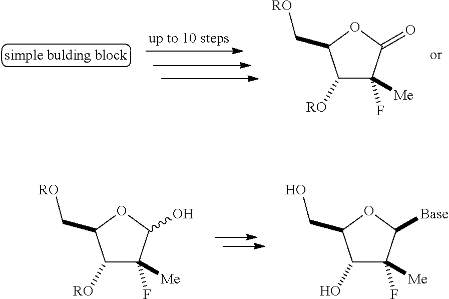

This approach is disclosed in patent applications WO 2006/031725 A, WO 2008/045419 A and in J. Org. Chem 2009, 74, page 6819. These references disclose the preparation of 2′-deoxy-2′ fuoro′-2′-C-methyl-D-ribofuranosyl nucleosides. The reagent HF-Et₃N is used for fluorination in a process with 10 to 11 steps and with an overall yield of from 2 to 12%.

WO 2008/090046 A discloses a process for the preparation of fluorinated nucleosides starting from the expensive fluorinated building block fluoropropionic acid. A mixture of ribonolactone diastereomers is obtained which have to be separated by crystallization in 6 to 7 steps leading to a low overall yield. Three additional steps are necessary to obtain the corresponding nucleoside.

WO 2007/075876 A discloses a process for the preparation of fluorinated nucleoside employing the expensive fluorinating reagent tris(dimethylamino)sulfonium difluorotrimethylsilicate (TASF). The process comprises 5 steps with an overall yield of 10% to obtain the ribonolactone, while three additional steps are necessary to obtain the corresponding nucleoside.

J. Am. Chem. Soc, 2014, 136, 16, pages 5900-5903, discloses a process for the preparation of fluorinated nucleosides. This route involves the use of a non-commercially available fluorinated building block, and the process leading to the nucleoside requires several steps with an overall yield of 25%. The process is not economic for industrial application due to the expensive reagents and the use of chiral catalysts.

The second approach entails the functionalization of a preformed (often natural) nucleoside precursor using a late-stage fluorination reaction according to the following scheme:

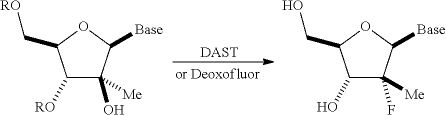

This approach represents a much faster process but mandatorily requires the use of either DAST (diethylaminosulfur trifluoride) or Deoxofluor® (bis(2-methoxyethyl)aminosulfur trifluoride) as fluorinating reagent. Both fluorinating reagents are expensive, hazardous, explosive, and, thus, incompatible with industrial synthesis. The fluorination yields are generally below 20%, and the reaction gives mixtures from which the desired product must be separated chromatographically. This approach is disclosed in WO 2013/096680 A, WO 2005/003147 A, and in Nucleosides, Nucleotides and Nucleic Acids, 2012, 31, page 277; Carbohydr. Chem. 2006, 25, page 461; J. Med. Chem. 2005, 48, page 5504; and in Nucleosides, Nucleotides and Nucleic Acids, 2011, 30, page 886.

Thus, there is a need for the provision of a novel process leading to 2′-fluorinated nucleosides in general, and to 2′-deoxy-2′-fluoro-2′-C-methyl-ribofuranosyl nucleosides in particular, which are suitable for industrial synthesis, i.e. which are cost-effective and which do not involve the use of toxic or hazardous reagents. Therefore, the problem underlying the present invention is the provision of a novel industrially applicable fluorination process for the preparation of 2′-fluoro nucleosides such as 2′-deoxy-2′-fluoro-2′-C-methyl-ribofuranosyl nucleosides which is carried out under mild and simple conditions, is economic and provides the corresponding 2′-fluoro nucleoside such as a 2′-deoxy-2′-fluoro-2′-C-methyl-ribofuranosyl nucleoside in good yields, leads to a product which can be easily purified and used directly in subsequent reactions such as for the preparation of 2′-deoxy-2-fluoro-2′C-methyluridine phosphoramidates such as sofosbuvir.

It was surprisingly found that the fluorination reaction of 2′-hydroxy-2′-methyl nucleosides of formula (I)

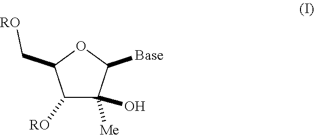

in which the primary and secondary hydroxyl groups (i.e. the 3′ and 5′ hydroxyl groups) are protected with an inert electron-withdrawing OH-protecting group R with XTalFluor leads to the formation of the corresponding fluorinated nucleosides in good yields. In addition, the formation of one undesired by-product is suppressed. Further, the process is carried out using the comparatively inexpensive reagent XTalFluor which is a stable, free-flowing solid that does not generate hazardous, corrosive hydrofluoric acid (HF), has significantly better thermal stability than other reagents such as DAST and shows no explosive behavior.

Process for Preparing Compound (II) or (III)

Therefore, the present invention relates to a process for the preparation of a compound of formula (II) or a compound of formula (III) including all isomers, stereoisomers, enantiomers and diastereomers thereof

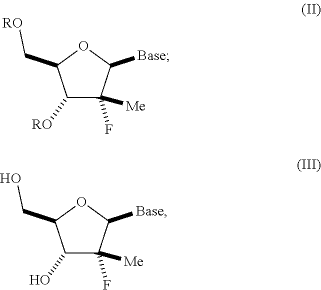

and salts thereof, preferably of the compound of formula (II) or the compound of formula (III), the process comprising
(i) providing a mixture comprising a compound of formula (I) or isomers, stereoisomers, diastereomers, enantiomers or salts thereof

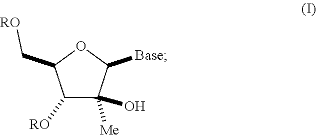

(ii) subjecting the mixture provided in (i) to fluorinating conditions in the presence of a fluorination agent selected from the group consisting of diethylamino(difluoro)sulfonium tetrafluoroborate and difluoro(morpholino)sulfonium tetrafluoroborate obtaining a mixture comprising a compound of formula (II) or isomers stereoisomers, diastereomers, enantiomers or salts thereof

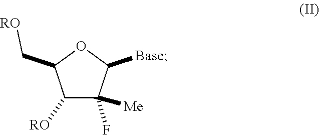

(iii) optionally subjecting the mixture obtained in (ii) to deprotection conditions, obtaining a mixture comprising the compound of formula (III) or isomers, stereoisomers, diastereomers, enantiomers or salts thereof

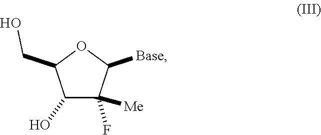

wherein at each occurrence R is an inert electron withdrawing OH protecting group; and Base is a purinyl residue or a pyrimidinyl residue linked to the furanose ring according to formulae (I), (II) and (III) through a carbon or a nitrogen atom. In the present context, the carbon or the nitrogen atom belong to the Base.

Thus, the present invention relates to a process for the preparation of a compound of formula (II) or a compound of formula (III) including all isomers, stereoisomers, enantiomers and diastereomers thereof

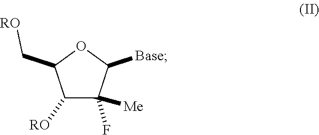

and salts thereof, preferably of the compound of formula (II), the process comprising
(i) providing a mixture comprising a compound of formula (I) or isomers, stereoisomers, diastereomers, enantiomers or salts thereof, preferably the compound of formula (I)

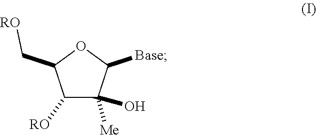

(ii) subjecting the mixture provided in (i) to fluorinating conditions in the presence of a fluorination agent selected from the group consisting of diethylamino(difluoro)sulfonium tetrafluoroborate and difluoro(morpholino)sulfonium tetrafluoroborate obtaining a mixture comprising a compound of formula (II) or isomers, stereoisomers, diastereomers, enantiomers or salts thereof, preferably the compound of formula (II)

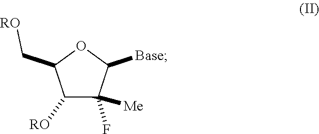

wherein at each occurrence R is an inert electron withdrawing OH protecting group; and Base is a purinyl residue or a pyrimidinyl residue linked to the furanose ring according to formulae (I), (II) and (III) through a carbon or a nitrogen atom.

Also, the present invention relates to a process for the preparation of a compound of formula formula (III) including all isomers, stereoisomers, enantiomers and diastereomers thereof

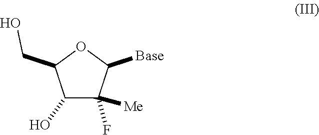

and salts thereof, preferably of the compound of formula (III), the process comprising
(i) providing a mixture comprising a compound of formula (I) or isomers, stereoisomers, diastereomers, enantiomers or salts thereof, preferably the compound of formula (I)

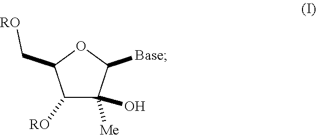

(ii) subjecting the mixture provided in (i) to fluorinating conditions in the presence of a fluorination agent selected from the group consisting of diethylamino(difluoro)sulfonium tetrafluoroborate and difluoro(morpholino)sulfonium tetrafluoroborate obtaining a mixture comprising a compound of formula (II) or isomers, stereoisomers, diastereomers, enantiomers or salts thereof, preferably the compound of formula (II)

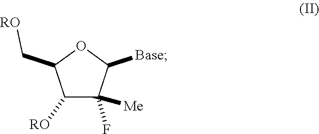

(iii) optionally subjecting the mixture obtained in (ii) to deprotection conditions, obtaining a mixture comprising the compound of formula (III) or isomers, stereoisomers, diastereomers, enantiomers or salts thereof, preferably the compound of formula (III)

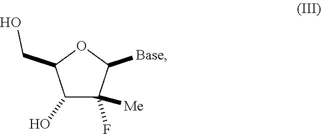

wherein at each occurrence R is an inert electron withdrawing OH protecting group; and Base is a purinyl residue or a pyrimidinyl residue linked to the furanose ring according to formulae (I), (II) and (III) through a carbon or a nitrogen atom.

It has been found that during the fluorination process commonly used electron-withdrawing protecting groups such as benzoyl (Bz), acetyl (Ac) and pivaloyl (Piv) react with at the tertiary carbon of the furanose ring, leading to the formation of undesired byproducts and lowering the overall reaction yield. In particular, these protecting groups engage in nucleophilic neighboring group participation (for example in the presence of DAST [see J. Carbohydrate Chem. 2001, 20, 431]) by reacting at the tertiary carbon of the furanose ring, which leads to the formation of undesired byproducts and lowers the overall reaction yield.

The term “inert electron-withdrawing hydroxyl protecting groups” in the context of the present invention refers to protecting groups which do not react at the neighboring tertiary carbon of the furanose ring, such as in position 2′, in particular these protecting groups do not engage in nucleophilic neighboring group participation by reacting at the tertiary carbon of the furanose ring, such as the tertiary carbon in position 2′. This lack of neighbouring group participation has been suggested to be due to stereoelectronic effects or geometrical constraints. (reference is made to page 11 in: Capon, B.; McManus, S. P. Neighbouring Group Participation; Plenum: New York, 1976 and in: Capon, B. Q. Rev. Chem. Soc. 1964, 18, 45-111 herein incorporated by reference),

It has also been found that during the fluorination process commonly used electron-donating protecting groups such as benzyl (Bn) and para-methoxy-benzyl (PMB) lead to formation of undesired by-products by rearrangements, in particular the hydride-shift induced rearrangements, lowering the overall reaction yield.

Thus, the use of the inert electron withdrawing hydroxyl protecting groups R of this invention results in combination with, for example, the fluorinating agents XTalFluor E (diethylamino(difluoro)sulfonium tetrafluoroborate) or XTalFluor M (difluoro(morpholino)sulfonium tetrafluoroborate) in higher-yielding fluorination reactions. In the fluorination process described in this invention, the use of XTalFluor E or XTalFluor M is preferred, the use of XTalFluor E is more preferred.

The fluorination reaction using fluorinating agents known in the art (such as e.g. DAST) in combination with the inert electron withdrawing hydroxyl protecting groups R of this invention leads to low reaction yields, as does the fluorination reaction with XTal Fluor E and M in combination with commonly used electron-withdrawing protecting groups such as benzoyl (Bz), acetyl (Ac) and pivaloyl (Piv) that react with the tertiary carbon of the furanose ring. It is the combination of the fluorinating agent XTal Fluor with the inert electron withdrawing hydroxyl protecting groups R of this invention that results in significantly increased reaction yields.

More preferably, the present invention relates to the above disclosed process wherein the inert electron withdrawing hydroxyl protecting group R is selected from the group consisting of X₃₋ₙHₙCC(O) wherein X is halogen and n is 0, 1, or 2; or

R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl); or

R is selected from SO₂Ph or SO₂-o-CF₃-Ph (ortho-trifluoromethylphenyl); or

R is

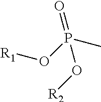

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or

R is selected from CH═CH₂—CO₂R₃ or C(O)—CH₂—CO₂R₃ wherein R₃ is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group selected from C(O), C(O)—(CH₂)ₜ—CO or

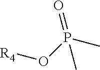

wherein R₄ is selected from the group consisting of alkyl, aryl and cycloalkyl and

wherein t is 1 or 2.

Regarding R₁, when R₁ is alkyl, the alkyl is preferably C₁-C₆ alkyl, more preferably C₁-C₄ alkyl, even more preferably C₁-C₂ alkyl; when R₁ is an aryl, aryl is preferably selected from phenyl or naphtyl, more preferably is phenyl.

Regarding R₂, when R₂ is alkyl, the alkyl is preferably is C₁-C₆ alkyl, more preferably C₁-C₄ alkyl, even more preferably C₁-C₂ alkyl, when R₂ is cycloalkyl, cycloalkyl is preferably a C₃-C₆ cycloalkyl, more preferably is a C₅-C₆ cycloalkyl; when R₂ is an aryl, aryl is preferably selected from phenyl or naphtyl, more preferably phenyl.

Regarding R₃, when R₃ is alkyl, the alkyl is preferably is C₁-C₆ alkyl, more preferably C₁-C₄ alkyl, even more preferably C₁-C₂ alkyl, when R₃ is cycloalkyl, cycloalkyl is preferably a C₃-C₆ cycloalkyl, more preferably is a C₅-C₆ cycloalkyl; when R₃ is an aryl, aryl is preferably selected from phenyl or naphtyl, more preferably phenyl.

Regarding R₄, when R₄ is alkyl, the alkyl is preferably is C₁-C₆ alkyl, more preferably C₁-C₄ alkyl, even more preferably C₁-C₂ alkyl, when R₄ is cycloalkyl, cycloalkyl is preferably a C₃-C₆ cycloalkyl, more preferably is a C₅-C₆ cycloalkyl; when R₄ is an aryl, aryl is preferably selected from phenyl or naphtyl, more preferably phenyl.

Regarding q, q is preferably selected from 2, 3 and 4.

Even more preferably, the present invention relates to said process wherein the inert electron withdrawing hydroxyl protecting group R is selected from the group consisting of X₃₋ₙHₙCC(O) wherein X is halogen and n is 0, 1, or 2; or

R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl).

Most preferably, the present invention relates to said process wherein the inert electron withdrawing hydroxyl protecting group R is selected from the group consisting of X₃₋ₙHₙCC(O) wherein X is halogen and n is 0, 1, or 2. Most preferably, the present invention relates to said process wherein the inert electron withdrawing hydroxyl protecting group R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl).

Such a protecting group can be a halogenated ester of the general formula X₃₋ₙHₙCC(O) wherein X is halogen and n is 0, 1, or 2 or a sulfonyl-containing group selected from the group consisting of SO₂Ph, SO₂Me, SO₂-p-Me-Ph (tosyl), SO₂-p-NO₂-Ph (para-nosyl), SO₂-o-NO₂-Ph (ortho-nosyl), SO2-o-CF3-Ph (ortho-trifluoromethylphenyl) and SO₂CF₃ (triflyl). In the context of the present invention the term “halogen” refers to halogen atoms such as I, Br, Cl and F.

Preferably, such a protecting group can be a halogenated ester of the general formula X₃₋ₙHₙCC(O) wherein X is halogen and n is 0, 1, or 2 or a sulfonyl-containing group selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).

More preferably, the inert electron withdrawing hydroxyl protecting group R is selected from the group consisting of F₃CC(O), Cl₃CC(O), ClH₂CC(O), Cl₂HCC(O), F₂HCC(O), FH₂CC(O) and SO₂Me. Even more preferably is selected from Cl₃CC(O).

Regarding Base, the present invention relates to a process wherein Base is selected from the group consisting of uridine, protected uridine, thymine, protected thymine, cytidine, protected cytidine, adenosine, protected adenosine, guanine and protected guanine. More preferably Base is selected from the group consisting of uridine, thymine, cytidine, adenosine, guanine. More preferably, Base is uridine.

Preferably, the present invention relates to a process wherein the compound of formula (I) is a compound of any of formulae (I-1) to (I-13)

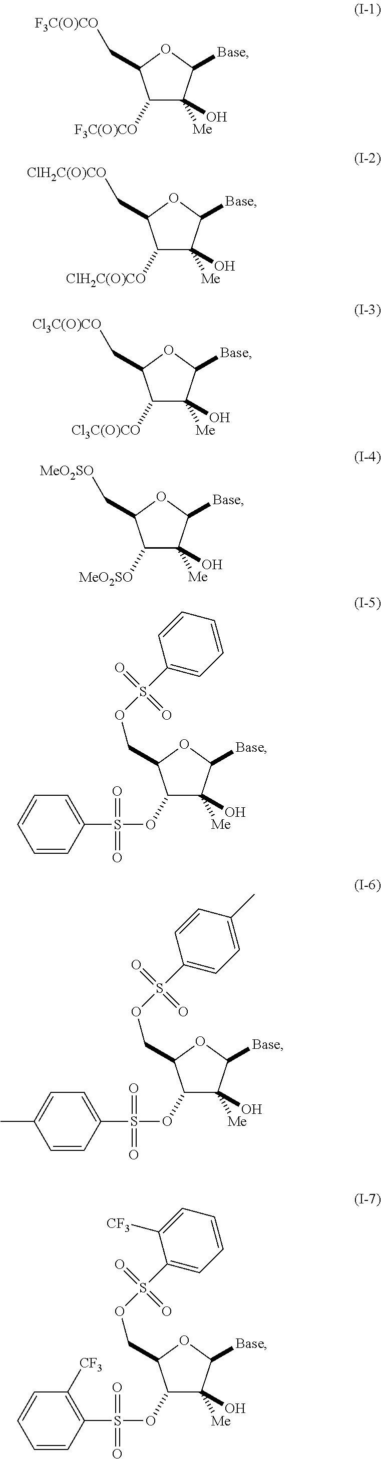

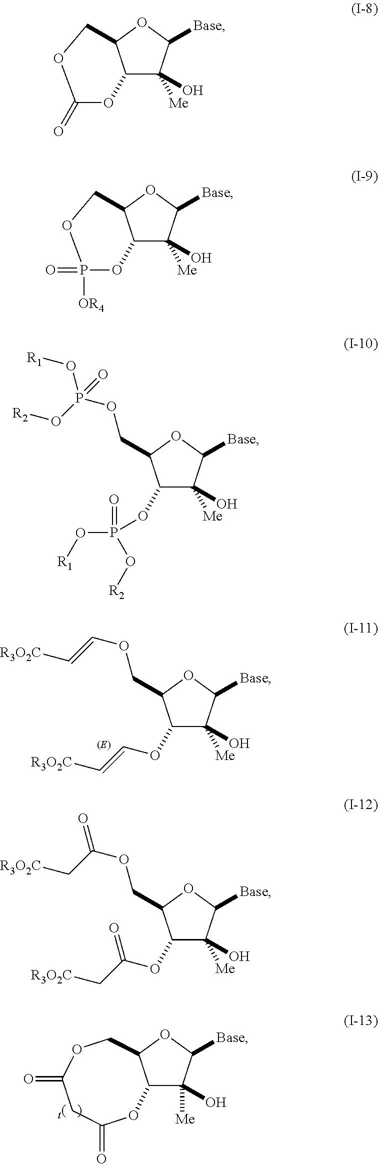

wherein in the formulae Base, R₁, R₂, R₃, R₄ and t are as defined above.

More preferably, the present invention relates to a process wherein the compound of formula (I) is a compound of formula (I-1) or (I-2) or (I-3) or (I-4)

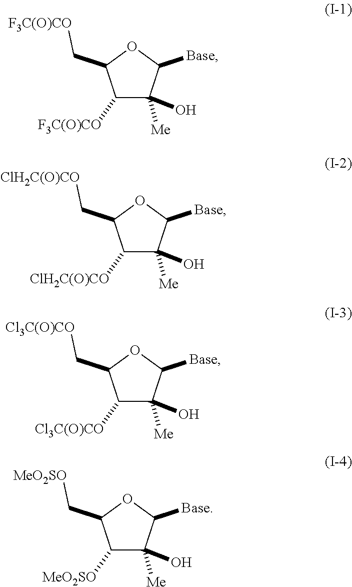

Preferably, the present invention relates to a process wherein the compound of formula (II) is a compound of any formulae (II-1) to (II-13)

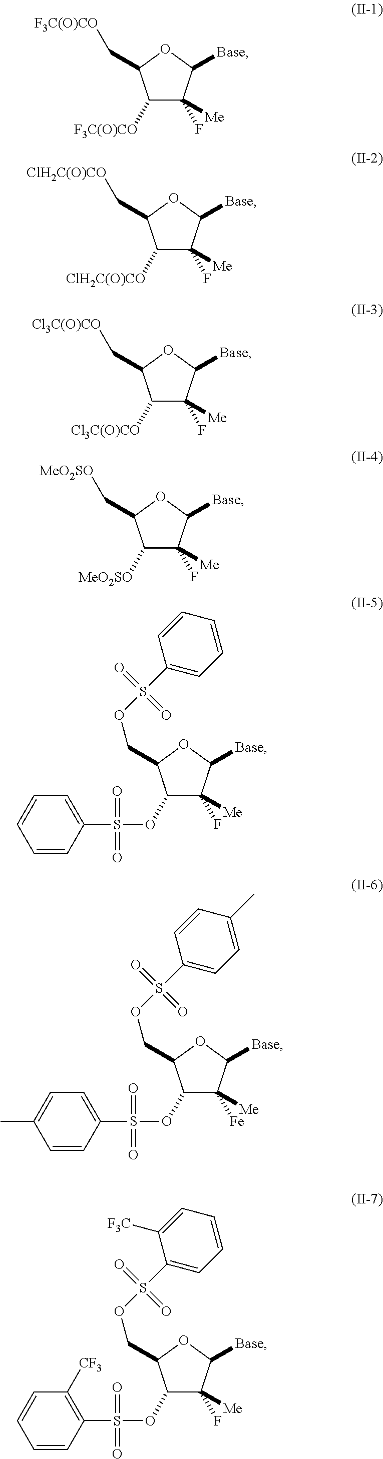

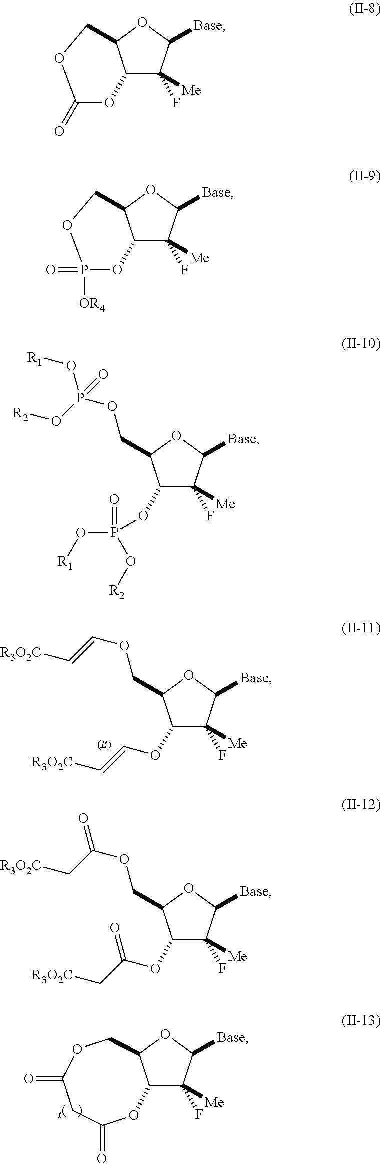

wherein in the formulae Base R₁, R₂, R₃, R₄ and t are as defined above.

More preferably, the present invention relates to a process wherein the compound of formula II is a compound of formula (II-1) or (II-2) or (II-3) or (II-4)

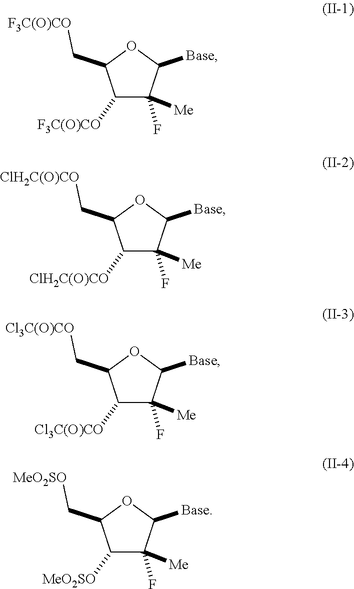

Even more preferably, the present invention relates to a process wherein the compound of formula (I) is a compound of any of formula (I-1′) to (I-13′)

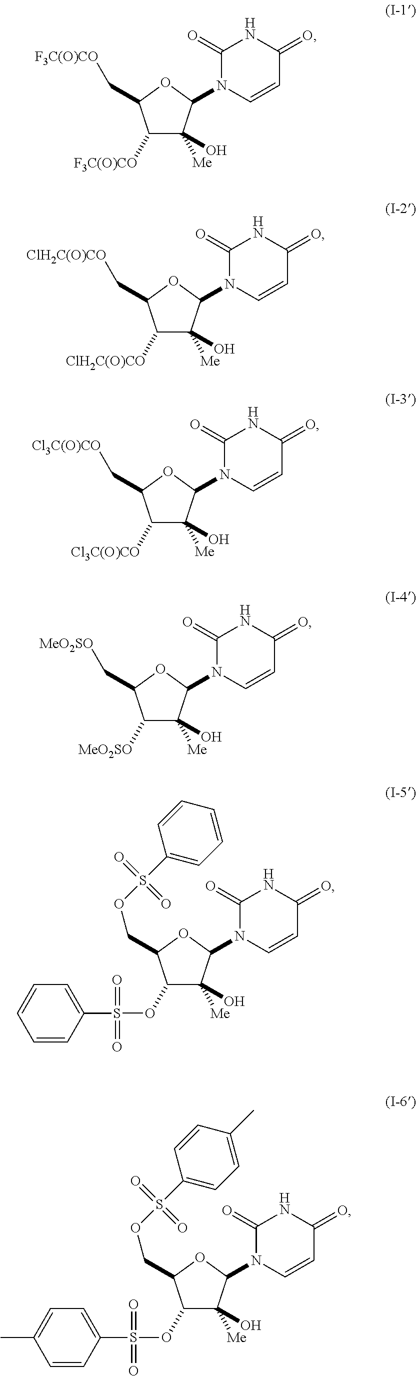

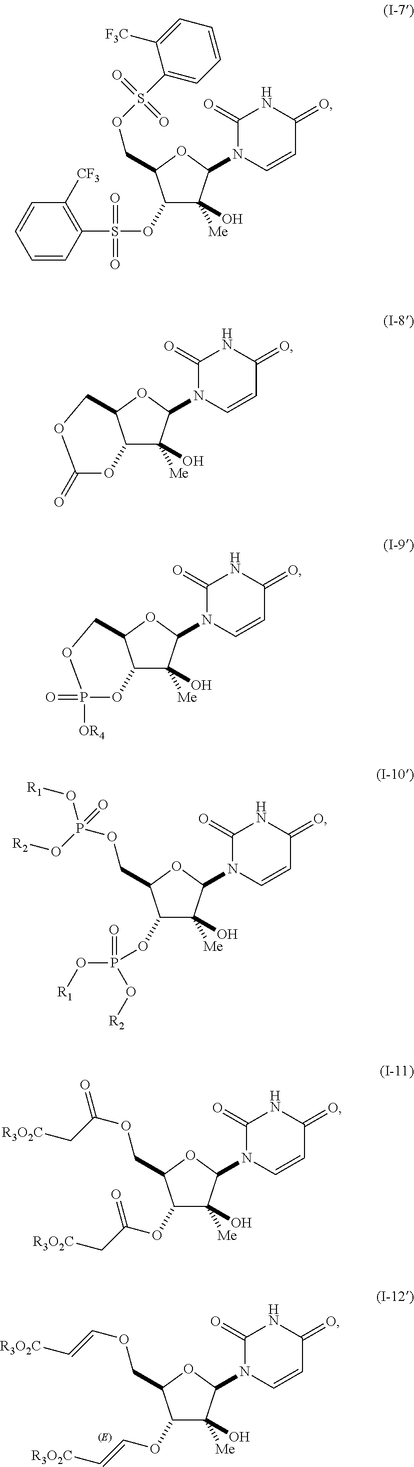

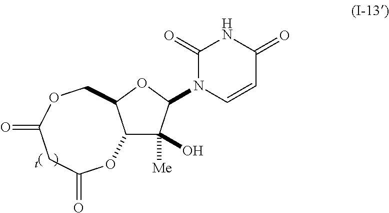

wherein in the formulae Base, R₁, R₂, R₃, R₄ and t are as defined above. Even more preferably, the present invention relates to a process wherein the compound of formula (I) is a compound of formula (I-1′) or (I-2′) or (I-3′) or (I-4′)

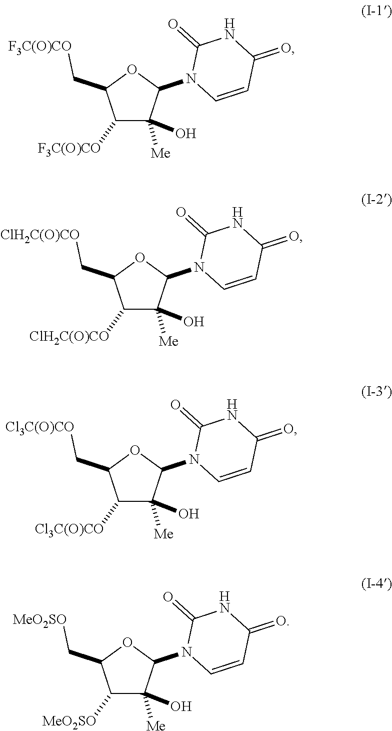

Even more preferably, the present invention relates to a process wherein the compound of formula (I) is a compound of formula (I-1′)

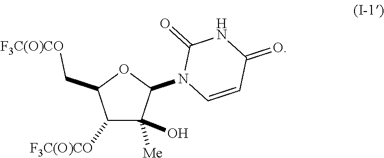

Even more preferably, the present invention relates to a process wherein the compound of formula (I) is a compound of formula (I-2′)

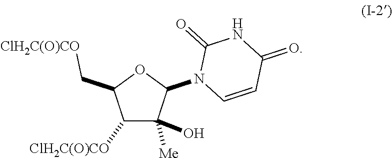

Even more preferably, the present invention relates to a process wherein the compound of formula (I) is a compound of formula (I-3′)

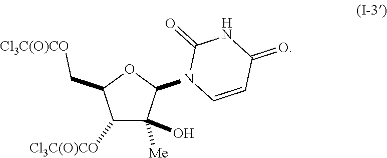

Even more preferably, the present invention relates to a process wherein the compound of formula (I) is a compound of formula (I-4′)

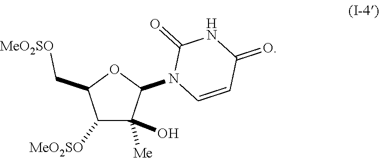

More preferably, the present invention relates to a process wherein the compound of formula (II) is a compound of any of formulae (II-1′) to (II-13′)

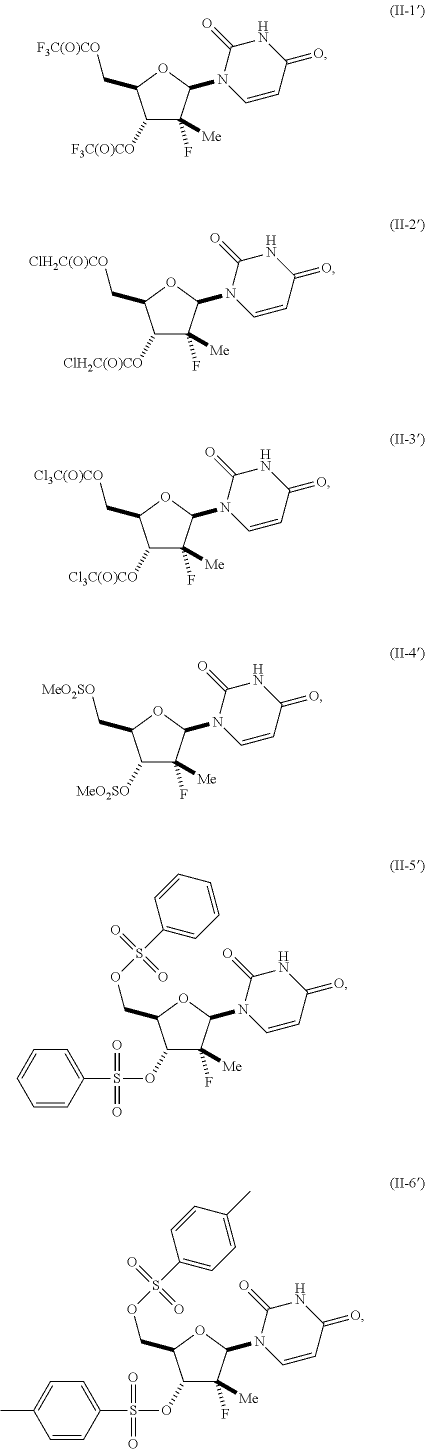

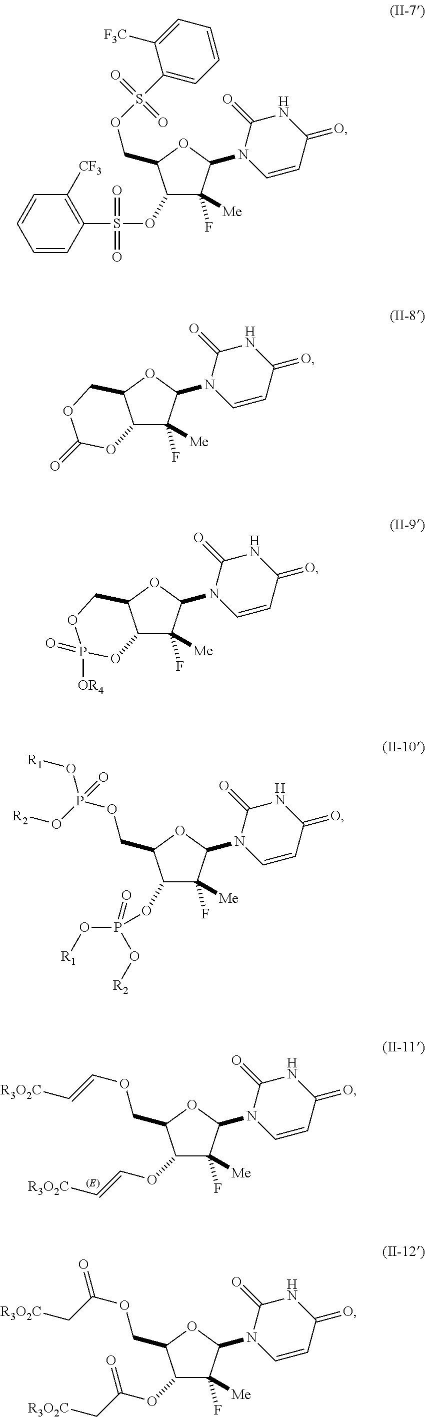

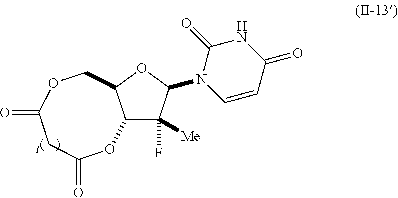

wherein in the formulae Base, R₁, R₂, R₃, R₄ and t are as defined above.

Even more preferably, the present invention relates to a process wherein the compound of formula (II) is a compound of formula (II-1′) or (II-2′) or (II-3′) or (II-4′)

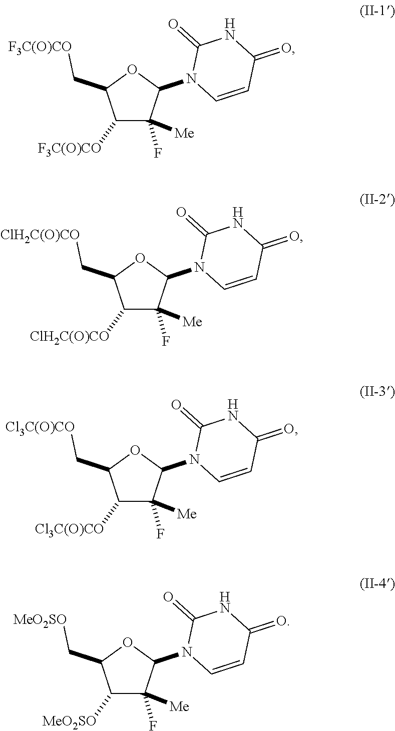

Even more preferably, the present invention relates to a process wherein the compound of formula (II) is a compound of formula (II-1′)

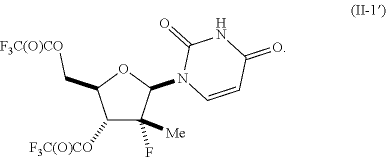

Even more preferably, the present invention relates to a process wherein the compound of formula (II) is a compound of formula (II-2′)

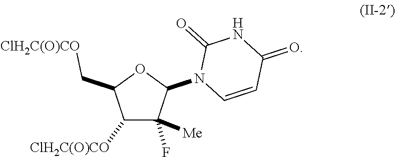

Even more preferably, the present invention relates to a process wherein the compound of formula (II) is a compound of formula (II-3′)

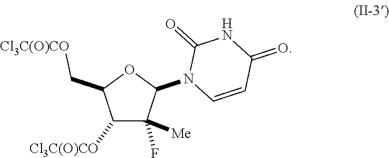

Even more preferably, the present invention relates to a process wherein the compound of formula (II) is a compound of formula (II-4′)

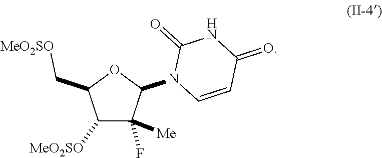

More preferably, the present invention relates to a process wherein the compound of formula (III) is the compound of formula (III)

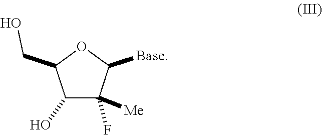

More preferably, the present invention relates to a process wherein the compound of formula (III) is the compound of formula (III′)

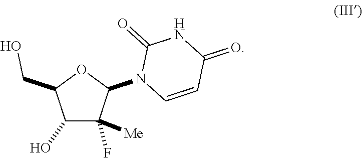 [SMILES: C[C@@]1(F)[C@H](O)[C@@H](CO)O[C@H]1n1ccc(=O)[nH]c1=O]

According to the present invention, it is preferred that the mixture provided in (i) comprises, in addition to the compound of formula (I), one or more solvents. Preferably, the one or more solvents are organic solvents. More preferably, the one or more organic solvents are one or more aprotic organic solvents. More preferably, the one or more organic solvents are one or more apolar aprotic organic solvents.

More preferably, the one or more organic solvents are selected from the group consisting of CH₂Cl₂, dichloroethane, chloroform, toluene, acetone, acetonitrile, 1,4-dioxane, tetrahydrofuran (THF), methyl tetrahydrofuran, methyl tert-butyl ether, methyl ethyl ketone, ethyl acetate, butyl acetate, nitromethane and a mixture of two or more thereof. More preferably the one or more organic solvents are selected from the group consisting of CH₂Cl₂, dichloroethane, chloroform, toluene, tetrahydrofuran, methyl tert-butyl ether, 1,4-dioxane, nitromethane and a mixture of two or more thereof. More preferably, the solvent is CH₂Cl₂ or tetrahydrofuran. More preferably, the solvent is CH₂Cl₂. According to the present invention, it is preferred that the solvent is anhydrous.

According to the present invention, it is preferred that the mixture provided in (i) comprises, in addition to the compound of formula (I) and preferably in addition to the one or more solvents or organic solvents, one or more organic bases. No specific limitation exists with regard to the chemical nature of the one or more bases provided that the reaction according to (ii) can be carried out, preferably in the one or more solvents mentioned above. Preferably the one or more organic bases are tertiary nitrogen bases. More preferably, the one or more bases are selected from the group consisting of triethylamine, pyridine, N,N′-diisopropylethylamine, 1,8-diazabicycloundec-7-ene, quinoline, isoquinoline, acridine, pyrazine, and imidazole, preferably one or more of triethylamine, N,N′-diisopropylethylamine, 1,8-diazabicycloundec-7-ene, and pyridine and a mixture of two or more thereof. More preferably, the base is triethylamine.

Regarding the molar ratio of the one or more bases relative to the compound of formula (I), no specific limitation exists provided that in (ii), the compound of formula (II) is obtained. Preferably, in the mixture provided in (i), the one or more bases and the compound of formula (I) are present in a molar ratio of the one or more bases relative to the compound of formula (I) in the range of from 0.1:1 to 3:1, preferably in the range of from 0.75:1 to 1.5:1, more preferably in the range of from 0.95:1 to 1.05:1. If more than one base is comprised in the mixture, the molar ratios relate to the total molar amount of all bases.

According to the present invention, it is preferred that the mixture provided in (i) comprises, in addition to the compound of formula (I) and preferably in addition to the one or more solvents or to the one or more organic bases, an agent selected from the group consisting of triethylamine trihydrofluoride (TEA 3HF), triethylamine dihydrofluoride (TEA 2HF), diazabicycloundec-7-ene (DBU), and a mixture of two or more thereof. Preferably, the agent is triethylamine trihydrofluoride or triethylamine dihydrofluoride.

Regarding the molar ratio of the agent relative to the compound of formula (I), no specific limitation exists provided that in (ii), the compound of formula (II) is obtained. Preferably, in the mixture provided in (i), the agent and the compound of formula (I) are present in a molar ratio of the agent relative to the compound of formula (I) in the range of from 0.1:1 to 3:1, preferably in the range of from 1.75:1 to 2.5:1, more preferably in the range of from 1.95:1 to 2.05:1.

According to the present invention, it is preferred that the mixture provided in (i) is provided in an inert gas atmosphere, preferably in an inert atmosphere comprising nitrogen. Therefore, according to the present invention, it is preferred that the mixture provided in (i) comprises, in addition to a compound of formula (I), one or more solvents and the agent, or the one or more solvents and the agent and the one or more bases.

Fluorinating agent is selected from the group consisting of XTalFluor E (diethylamino(difluoro)sulfoniumtetrafluoroborate) and XTalFluor M (difluoro(morpholino)sulfonium tetrafluoroborate). Preferably, the fluorinating agent is XTalFluor E (diethylamino(difluoro)sulfoniumtetrafluoroborate).

According to the present invention, it is preferred that the mixture provided in (i) comprises, in addition to the compound of formula (I) and preferably in addition to the one or more solvents or to the one or more organic bases or to the agent, XTalFluor E (diethylamino(difluoro)sulfonium tetrafluoroborate) or XTalFluor M (difluoro(morpholino)sulfonium tetrafluoroborate). Preferably, the mixture provided in (i) comprises, in addition to the compound of formula (I) and preferably in addition to the one or more solvents or to the one or more organic bases or to the agent, XTalFluor E (diethylamino(difluoro)sulfonium tetrafluoroborate).

Therefore, according to the present invention, it is preferred that the mixture provided in (i) comprises in addition to a compound of formula (I) XTalFluor E (diethylamino(difluoro)sulfonium tetrafluoroborate) or XTalFluor M (difluoro(morpholino)sulfonium tetrafluoroborate), the one or more solvents, the agent and optionally the one or more bases.

Preferably, the mixture provided in (i) comprises in addition to a compound of formula (I) XTalFluor E (diethylamino(difluoro)sulfonium tetrafluoroborate), the one or more solvents, the agent and optionally the one or more bases.

Regarding the molar ratio of XTalFLuor E or XTalFluor M relative to the compound of formula (I), no specific limitation exists provided that in (ii), the compound of formula (II) is obtained. Preferably, the fluorinating agent is employed in an amount so that prior to (ii) XTalFLuor E (diethylamino(difluoro)sulfonium tetrafluoroborate) or XTalFluor M (difluoro(morpholino)sulfonium tetrafluoroborate) is present in a molar ratio of XTalFLuor E or XTalFluor M relative to the compound of formula (I) in the range of from 0.1:1 to 3:1, preferably in the range of from 1.25:1 to 2.0:1, more preferably in the range of from 1.45:1 to 1.65:1.

In (ii), the mixture provided in (i) is subjected to fluorinating conditions in the presence of XTalFluor E (diethylamino(difluoro)sulfonium tetrafluoroborate) or XTalFluor M (difluoro(morpholino)sulfonium tetrafluoroborate), thereby obtaining a mixture comprising a compound of formula (II)

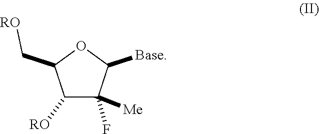

Regarding the reaction temperature in (ii), no specific limitation exists provided that the compound of formula (II) is obtained. Preferably, the temperature during (ii) is in the range of from −80 to 40° C., more preferably in the range of from 20 to 30° C., more preferably in the range of from 20 to 25° C.

Regarding the time during which the mixture is subjected to the reaction conditions, no specific limitation exists provided that in (ii), the compound of formula (II) is obtained. Preferably, according to (ii) the mixture is subjected to the fluorination conditions for a period of time in the range of from 0.5 to 24 h, more preferably in the range of from 0.5 to 2 h, more preferably in the range of from 0.5 to 1.5 h.

Preferably, prior to (iii), the compound of formula (II) is separated from the mixture obtained in (ii), and the process of the present invention further comprises, (ii′) separating the compound of formula (II) from the mixture obtained in (ii).

More preferably, the separating in (ii′) comprises

(ii′-1) extracting the compound of formula (II) from the mixture obtained in (ii);

(ii′-2) separating the compound of formula (II) from the mixture obtained in (ii′-1).

More preferably, the separating according to (ii′) or the separating according to (ii′-2) comprises filtration, centrifugation, drying, or a combination of two or more thereof.

In (iii), the mixture obtained in (ii) is optionally subjected to deprotection conditions, obtaining a mixture comprising a compound of formula (III)

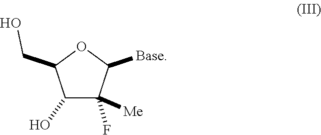

Preferably, subjecting the mixture obtained in (ii) to deprotection conditions further comprises adding to the mixture obtained in (ii) one or more deprotection reagents, preferably selected from the group consisting of water, a mixture of NH₃ and MeOH, and a mixture of NaOMe and MeOH.

Regarding the reaction temperature in (iii), no specific limitation exists provided that in (iii), the compound of formula (III) is obtained. Preferably, the deprotection conditions in (iii) comprise a temperature of the mixture in the range of from 15 to 35° C., preferably in the range of from 20 to 30° C., more preferably in the range of from 20 to 25° C.

Regarding the time during which the mixture is subjected to the reaction conditions in (iii), no specific limitation exists provided that in (iii), the compound of formula (III) is obtained.

Preferably, in (iii) the mixture is subjected to the deprotection conditions for a period of time in the range of from less than 1 to 120 min or from 1 to 120 min, preferably in the range of from less than 1 to 50 min or from 1 to 50 min.

Preferably, the compound of formula (III) is separated after (iii) from the mixture obtained in (iii), and the process of the present invention further comprises,

(iv) separating the compound of formula (III) from the mixture obtained in step (iii).

More preferably, the separating in (iv) comprises

(iv-1) extracting the compound of formula (III) from the mixture obtained in (iii), and

(iv-2) separating the compound of formula (III) from the mixture obtained in (iv-1).

More preferably, the separating according to (iv) or the separating according to (iv-2) comprises filtration, centrifugation, drying, or a combination of two or more thereof.

More preferably, the separating according to (iv) or according to (iv-2) comprises

(iv-1′) crystallizing the compound of formula (III) from the mixture obtained in (iv) or in (iv-2),

(iv-2′) separating the compound of formula (III) in the mixture obtained from (iv-1′) from its mother liquor.

During the separating according to (iv) or according to (iv-2), the compound of formula (III) is preferably crystallized. Preferably, the crystallizing according to (iv-1′) comprises seeding with seeds of the compound of formula (III). Preferably, crystallizing according to (iv-1′) is carried out in a suitable solvent which is preferably selected from the group consisting of ethyl acetate, isopropanol and tetrahydrofuran.

Process for Preparing Compound (I)

Further, the present invention relates to a process as described above which further comprises providing the mixture according to (i) by a process comprising
(a) providing a mixture comprising a compound of formula (IV)

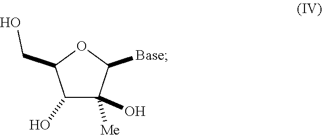

(b) subjecting the mixture provided in (a) to protection conditions in the presence of an OH-protecting agent comprising an inert electron-withdrawing OH-protecting group R, obtaining a mixture comprising the compound of formula (I)

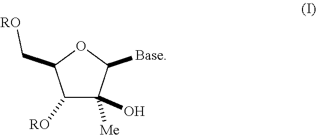

Further, the present invention relates to a process for the preparation of a mixture comprising a compound of formula (I) as described above comprising
(a) providing a mixture comprising a compound of formula (IV)

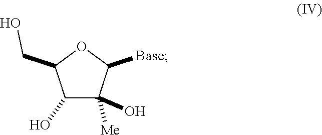

(b) subjecting the mixture provided in (a) to protection conditions in the presence of an OH-protecting agent comprising an inert electron-withdrawing OH-protecting group R, obtaining a mixture comprising a compound of formula (I)

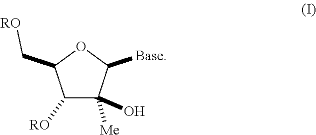

Further, the present invention relates to a process for the preparation a compound of formula (I) as described above comprising
(a) providing a mixture comprising a compound of formula ((IV)

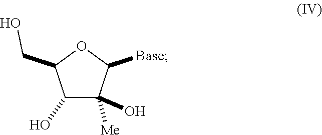

(b) subjecting the mixture provided in (a) to protection conditions in the presence of an OH-protecting agent comprising an inert electron-withdrawing OH-protecting group R, obtaining a compound of formula (I)

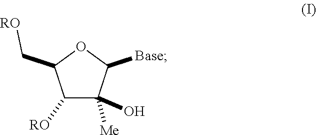

wherein at each occurrence R is an inert electron withdrawing OH protecting group; and Base is a purinyl residue or a pyrimidinyl residue linked to the furanose ring according to formulae (I) and (IV) through a carbon or a nitrogen atom.

It has been found that during the fluorination process commonly used electron-withdrawing protecting groups such as benzoyl (Bz), acetyl (Ac) and pivaloyl (Piv) react at the tertiary carbon of the furanose ring, leading to the formation of undesired byproducts and lowering the overall reaction yield. In particular, it has been suggested and confirmed that these groups engage in a nucleophilic neighboring group participation reacting at the tertiary carbon of the furanose ring, leading to the formation of undesired byproducts and lowering the overall reaction yield. It has also been found that the use of electron-donating protecting groups such as benzyl (Bn) and para-methoxy-benzyl (PMB) lead to formation of undesired by-products by rearrangements, in particular hydride-shift induced rearrangements, lowering the overall reaction yield.

The term “inert electron-withdrawing hydroxyl protecting groups” in the context of the present invention refers to protecting groups which do not react at the neighboring tertiary carbon of the furanose ring, such as in position 2′, in particular these protecting groups do not engage in nucleophilic neighboring group participation by reacting at the tertiary carbon of the furanose ring, such as the tertiary carbon in position 2′. This lack of neighbouring group participation has been suggested to be due to stereoelectronic effects or geometrical constraints. (reference is made to page 11 in: Capon, B.; McManus, S. P. Neighbouring Group Participation; Plenum: New York, 1976 and in: Capon, B. Q. Rev. Chem. Soc. 1964, 18, 45-111 herein incorporated by reference).

More preferably, the present invention relates to any of the aforementioned processes wherein the inert electron withdrawing hydroxyl protecting group R is selected from the group consisting of X₃₋ₙHₙCC(O) wherein X is halogen, wherein preferably halogen is Cl or F, more preferably halogen is Cl and n is 0, 1, or 2; or R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl) or R is selected from SO₂Ph or SO₂-o-CF₃-Ph (ortho-trifluoromethylphenyl); or R is

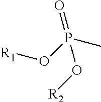

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or

R is selected from CH═CH₂—CO₂R₃ or C(O)—CH₂—CO₂R₃ wherein R₃ is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group selected from C(O), C(O)—(CH₂)ₜ—CO or

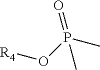

wherein R₄ is selected from the group consisting of alkyl, aryl and cycloalkyl and

wherein t is 1 or 2.

Regarding R₁, when R₁ is alkyl, the alkyl is preferably C₁-C₆ alkyl, more preferably C₁-C₄ alkyl, even more preferably C₁-C₂ alkyl; when R₁ is an aryl, aryl is preferably selected from phenyl or naphtyl, more preferably is phenyl.

Regarding R₂, when R₂ is alkyl, the alkyl is preferably is C₁-C₆ alkyl, more preferably C₁-C₄ alkyl, even more preferably C₁-C₂ alkyl, when R₂ is cycloalkyl, cycloalkyl is preferably a C₃-C₆ cycloalkyl, more preferably is a C₅-C₆ cycloalkyl; when R₂ is an aryl, aryl is preferably selected from phenyl or naphtyl, more preferably phenyl.

Regarding R₃, when R₃ is alkyl, the alkyl is preferably is C₁-C₆ alkyl, more preferably C₁-C₄ alkyl, even more preferably C₁-C₂ alkyl, when R₃ is cycloalkyl, cycloalkyl is preferably a C₃-C₆ cycloalkyl, more preferably is a C₅-C₆ cycloalkyl; when R₃ is an aryl, aryl is preferably selected from phenyl or naphtyl, more preferably phenyl.

Regarding R₄, when R₄ is alkyl, the alkyl is preferably is C₁-C₆ alkyl, more preferably C₁-C₄ alkyl, even more preferably C₁-C₂ alkyl, when R₄ is cycloalkyl, cycloalkyl is preferably a C₃-C₆ cycloalkyl, more preferably is a C₅-C₆ cycloalkyl; when R₄ is an aryl, aryl is preferably selected from phenyl or naphtyl, more preferably phenyl.

Regarding q, q is preferably selected from 2, 3 and 4.

Even more preferably, the present invention relates to any of the aforementioned processes wherein the inert electron withdrawing hydroxyl protecting group R is selected from the group consisting of X₃₋ₙHₙCC(O) wherein X is halogen, wherein preferably halogen is Cl or F, more preferably halogen is Cl and n is 0, 1, or 2; or R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).

Such a protecting group R can be a halogenated ester of the general formula X₃₋ₙHₙCC(O) wherein X is halogen and n is 0, 1, or 2 or a sulfonyl-containing group selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl). In the context of the present invention the term “halogen” refers to halogen atoms such as I, Br, Cl and F.

More preferably, the inert electron withdrawing hydroxyl protecting group R is selected from the group consisting of F₃CC(O), Cl₃CC(O), ClH₂CC(O), Cl₂HCC(O), F₂HCC(O), FH₂CC(O) and SO₂Me. Preferably, the inert electron withdrawing hydroxyl protecting group R is C(O)CCl₃, C(O)CF₃, C(O)CH₂Cl, more preferably C(O)CCl₃, C(O)CH₂Cl.

Regarding the OH-protecting agent comprising an inert electron-withdrawing OH-protecting group R, there is no particular limitation with respect to its nature provided that such agent is suitable for the introduction of the above-described inert electron withdrawing hydroxyl protecting group R. Preferably, the OH-protecting agent comprising an inert electron-withdrawing OH-protecting group R is Cl—C(O)CCl₃, Cl—C(O)CF₃, O(C(O)CF₃)₂Cl—C(O)CH₂Cl, O(C(O)CH₂Cl)₂Cl₂HCC(O)—Cl, F₂HCC(O)—Cl, FH₂CC(O)—Cl or Cl—SO₂Me, preferably the agent is Cl—C(O)CCl₃, O(C(O)CF₃)₂ or O(C(O)CH₂Cl)₂, Cl—SO₂Ar wherein Ar is p-NO₂Ph o-NO₂Ph or p-MePh, O(SO₂CF₃)₂, (R₁O)(R₂O)P(O)—Cl wherein R₁ and R₂ are defined as above.

Regarding the Base, the present invention relates to processes wherein the Base is selected from the group consisting of uridine, protected uridine, thymine, protected thymine, cytidine, protected cytidine, adenosine, protected adenosine, guanine and protected guanine. More preferably Base is selected from the group consisting of uridine, thymine, cytidine, adenosine, guanine. More preferably, the Base is uridine.

According to the present invention, it is preferred that the mixture provided in (a) comprises, in addition to the compound of formula (IV), one or more solvents. Preferably, the one or more solvents are organic solvents. More preferably, the one or more organic solvents are one or more polar organic solvents.

More preferably, the one or more organic solvents are selected from the group consisting of CH₂Cl₂, pyridine toluene, acetone, acetonitrile, dioxane, tetrahydrofuran (THF), methyl tetrahydrofuran, methyl ethyl ketone, ethyl acetate, butyl acetate, dimethylformamide and a mixture of two or more thereof. More preferably the solvent is CH₂Cl₂, tetrahydrofuran, pyridine, dimethylformamide and a mixture of two or more thereof. More preferably the solvent is pyridine or dimethylformamide. According to the present invention, it is preferred that the solvent be anhydrous.

According to the present invention, it is preferred that the mixture provided in (a) comprises, in addition to the compound of formula (IV) and preferably in addition to the one or more organic solvents, one or more organic or one or more inorganic bases or mixtures of two or more thereof. No specific limitation exists with regard to the chemical nature of the one or more bases provided that the reaction according to (b) can be carried out, preferably in the one or more solvents mentioned above.

When the mixture provided in (a) comprises, in addition to the compound of formula (IV) and preferably in addition to the one or more organic solvents, one or more inorganic bases it is preferred that the mixture provided in (a) comprises a carbonate, more preferably an alkali metal carbonate, more preferably sodium carbonate.

When the mixture provided in (a) comprises, in addition to the compound of formula (IV) and preferably in addition to the one or more organic solvents, one or more organic bases it is preferred that the mixture provided in (a) comprises one or more organic tertiary nitrogen bases.

Preferably the one or more bases are one or more selected from the group consisting of pyridine, 2,6 dimethylpyridine, triethylamine N,N′-diisopropylethylamine, 1,8-diazabicycloundec-7-ene, quinoline, isoquinoline, acridine, pyrazine, and imidazole, preferably one or more of triethylamine, N,N′-diisopropylethylamine, 1,8-diazabicycloundec-7-ene, pyridine and a mixture of two or more thereof. More preferably, the base is pyridine or 2,6 dimethylpyridine.

Regarding the molar ratio of the one or more bases relative to the compound of formula (IV), no specific limitation exists provided that in (b), the compound of formula (I) is obtained. Preferably, in the mixture provided in (a), the one or more bases and the compound of formula (IV) are present in a molar ratio of the one or more bases relative to the compound of formula (IV) in the range of from 3:1 to 30:1, preferably in the range of from 10:1 to 25:1, more preferably in the range of from 17:1 to 22:1. If more than one base is comprised in the mixture, the molar ratios relate to the total molar amount of all bases.

According to the present invention, it is preferred that the mixture provided in (a) comprises, in addition to the compound of formula (IV) and preferably in addition to the one or more solvents or to the one or more organic bases, a reagent selected from the group consisting of N,N-dialkylaminopyridines and pyridine. More preferably the N,N-dialkylaminopyridine is N,N-dimethylaminopyridine (DMAP).

Regarding the molar ratio of the reagent relative to the compound of formula (IV), no specific limitation exists provided that in (b) the compound of formula (I) is obtained. Preferably, in the mixture provided in (a), the reagent selected from the group consisting of N,N-dialkylaminopyridines and pyridine, preferably wherein the N,N-dialkylaminopyridine is N,N-dimethylaminopyridine (DMAP) and the compound of formula (IV) are present in a molar ratio of reagent selected from the group consisting of N,N-dialkylaminopyridines and pyridine, preferably wherein the N,N-dialkylaminopyridine is N,N-dimethylaminopyridine (DMAP) relative to the compound of formula (IV) in the range of from 0.1:1 to 0.6:1.

Therefore, according to the present invention, it is preferred that the mixture provided in (a) comprises in addition to a compound of formula (IV) one or more solvents and the reagent, or one or more solvents and the reagent and the one or more bases.

Regarding the OH-protecting agent comprising an inert electron-withdrawing OH-protecting group R, there is no particular limitation with respect to its nature provided that such agent is suitable for the introduction of the above-described inert electron withdrawing hydroxyl protecting group R. It is preferred that the protecting agent and the compound of formula (IV) are present in the reaction mixture provided in (a) prior to subjecting the mixture to the protecting conditions of (b).

Regarding the molar ratio of the OH-protecting agent comprising an inert electron-withdrawing OH-protecting group R and the compound of formula (IV), it is preferred that the protecting agent and the compound of formula (IV) are present in the reaction mixture provided in (a) prior to subjecting the mixture to the protecting conditions of (b) in a molar ratio of protecting agent relative to the compound of formula (IV) in the range of from 1:1 to 10:1, preferably in the range of from 2:1 to 9:1, more preferably in the range of from 2.5:1 to 7:1.

Therefore, according to the present invention, it is preferred that the mixture provided in (a) comprises in addition to a compound of formula (IV) one or more solvents, the reagent, the OH-protecting agent comprising an inert electron-withdrawing OH-protecting group R and optionally the one or more bases.

In (b), the mixture provided in (a) is subjected to protection conditions in the presence of an OH-protecting agent comprising an inert electron-withdrawing OH-protecting group R, thereby obtaining a mixture comprising the compound of formula (I). Regarding the reaction temperature in (b), no specific limitation exists provided that in (b) the compound of formula (I) is obtained. Preferably, the temperature during (b) is in the range of from 15 to 35° C., preferably in the range of from 20 to 30° C.

Regarding the time during which the mixture is subjected to the reaction conditions, no specific limitation exists provided that in (b), the compound of formula (I) is obtained. Preferably, according to (b) the mixture is subjected to the protection conditions for a period of time in the range of from 1 to 24 h, preferably in the range of from 2 to 20 h.

Preferably the compound of formula (I) is separated from the mixture obtained in (b), and the above-mentioned processes of the present invention further comprise (c) separating the compound of formula (I) from the mixture obtained in (b).

More preferably, the separating in (c) comprises

(c-1) extracting the compound of formula (I) from the mixture obtained in (b), and

(c-2) separating the compound of formula (I) from the mixture obtained in (c-1).

More preferably, the separating according to (c) or the separating according to (c-2) comprises filtration, centrifugation, drying, or a mixture of two or more thereof.

According to the present invention, it is preferred that the mixture provided in (a) is provided in an inert gas atmosphere, preferably in an inert atmosphere comprising nitrogen.

### Compound of Formula (I)

Yet further, it is provided a compound of formula (I) or isomers, stereoisomers, diastereomers, enantiomers or salts thereof, preferably the compound of formula (I)

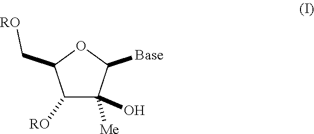

wherein at each occurrence,

R is an inert electron withdrawing OH protecting group; and

Base is a purinyl residue or a pyrimidinyl residue linked to the furanose ring according to formula (I) through a carbon or a nitrogen atom.

As described above, the term “inert electron-withdrawing hydroxyl protecting groups” in the context of the present invention refers to protecting groups which do not react at the neighboring tertiary carbon of the furanose ring, such as in position 2′, in particular these protecting groups do not engage in nucleophilic neighboring group participation by reacting at the tertiary carbon of the furanose ring, such as the tertiary carbon in position 2′. This lack of neighbouring group participation has been suggested to be due to stereoelectronic effects or geometrical constraints. (reference is made to page 11 in: Capon, B.; McManus, S. P. Neighbouring Group Participation; Plenum: New York, 1976 and in: Capon, B. Q. Rev. Chem. Soc. 1964, 18, 45-111 herein incorporated by reference).

Compounds of formula (I) are preferably compounds of formula (I) wherein R is selected from the group consisting of X₃₋ₙHₙCC(O) wherein X is halogen, preferably Cl or F, more preferably Cl and n is 0, 1, or 2; or R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl) or

R is selected from SO₂Ph or SO₂-o-CF₃-Ph (ortho-trifluoromethylphenyl); or

R is

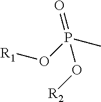

wherein R₁ and R₂ are independently selected from alkyl, aryl or R₁ and R₂ taken together are a (CH₂)_{q} group that forms a ring with the oxygen atoms to which R₁ and R₂ are bound and

wherein q is 2, 3, 4, 5, 6, 7; or

R is selected from CH═CH₂—CO₂R₃ or C(O)—CH₂—CO₂R₃ wherein R₃ is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group selected from C(O), C(O)—(CH₂)ₜ—CO or

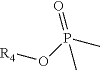

wherein R₄ is selected from the group consisting of alkyl, aryl and cycloalkyl and

wherein t is 1 or 2.

Regarding R₁, when R₁ is alkyl, the alkyl is preferably C₁-C₆ alkyl, more preferably C₁-C₄ alkyl, even more preferably C₁-C₂ alkyl; when R₁ is an aryl, aryl is preferably selected from phenyl or naphtyl, more preferably is pheny.

Regarding R₂, when R₂ is alkyl, the alkyl is preferably is C₁-C₆ alkyl, more preferably C₁-C₄ alkyl, even more preferably C₁-C₂ alkyl, when R₂ is cycloalkyl, cycloalkyl is preferably a C₃-C₆ cycloalkyl, more preferably is a C₅-C₆ cycloalkyl; when R₂ is an aryl, aryl is preferably selected from phenyl or naphtyl, more preferably phenyl.

Regarding R₃, when R₃ is alkyl, the alkyl is preferably is C₁-C₆ alkyl, more preferably C₁-C₄ alkyl, even more preferably C₁-C₂ alkyl, when R₃ is cycloalkyl, cycloalkyl is preferably a C₃-C₆ cycloalkyl, more preferably is a C₅-C₆ cycloalkyl; when R₃ is an aryl, aryl is preferably selected from phenyl or naphtyl, more preferably phenyl.

Regarding R₄, when R₄ is alkyl, the alkyl is preferably is C₁-C₆ alkyl, more preferably C₁-C₄ alkyl, even more preferably C₁-C₂ alkyl, when R₄ is cycloalkyl, cycloalkyl is preferably a C₃-C₆ cycloalkyl, more preferably is a C₅-C₆ cycloalkyl; when R₄ is an aryl, aryl is preferably selected from phenyl or naphtyl, more preferably phenyl.

Regarding q, q is preferably selected from 2, 3 and 4.

Even more preferably, the present invention relates to said compound of formula (I) wherein the inert electron withdrawing hydroxyl protecting group R is selected from the group consisting of X₃₋ₙHₙCC(O) wherein X is halogen and n is 0, 1, or 2; or

R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl).

Such a protecting group R can be a halogenated ester of the general formula X₃₋ₙHₙCC(O) wherein X is halogen and n is 0, 1, or 2 or a sulfonyl-containing group selected from the group consisting of SO₂Ph, SO₂Me, SO₂-p-Me-Ph (tosyl), SO₂-p-NO₂-Ph (para-nosyl), SO₂-o-NO₂-Ph (ortho-nosyl), SO2-o-CF3-Ph (ortho-trifluoromethylphenyl) and SO₂CF₃ (triflyl).

Compounds of formula (I) are more preferably compounds of formula (I) wherein R is selected from the group consisting of F₃CC(O), Cl₃CC(O), ClH₂CC(O), Cl₂HCC(O), F₂HCC(O), FH₂CC(O) and SO₂Me. Even more preferably is selected from Cl₃CC(O) or Cl₂HCC(O).

Regarding to the radical Base, Base is a purinyl residue or a pyrimidinyl residue linked to the furanose ring according to formula (I) through a carbon or nitrogen atom; preferably, Base is selected from the group consisting of uridine, protected uridine, thymine, protected thymine, cytidine, protected cytidine, adenosine, protected adenosine, guanine, protected guanine; more preferably Base is selected from the group consisting of uridine, thymine, cytidine, adenosine, guanine; more preferably Base is uridine.

More preferred compounds of formula (I) are compounds having any of formulae (I-1) to (I-13)

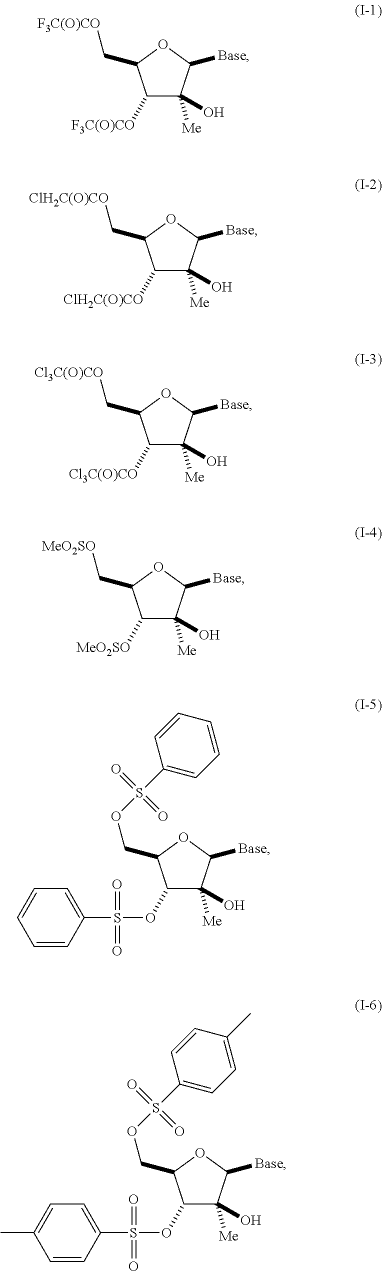

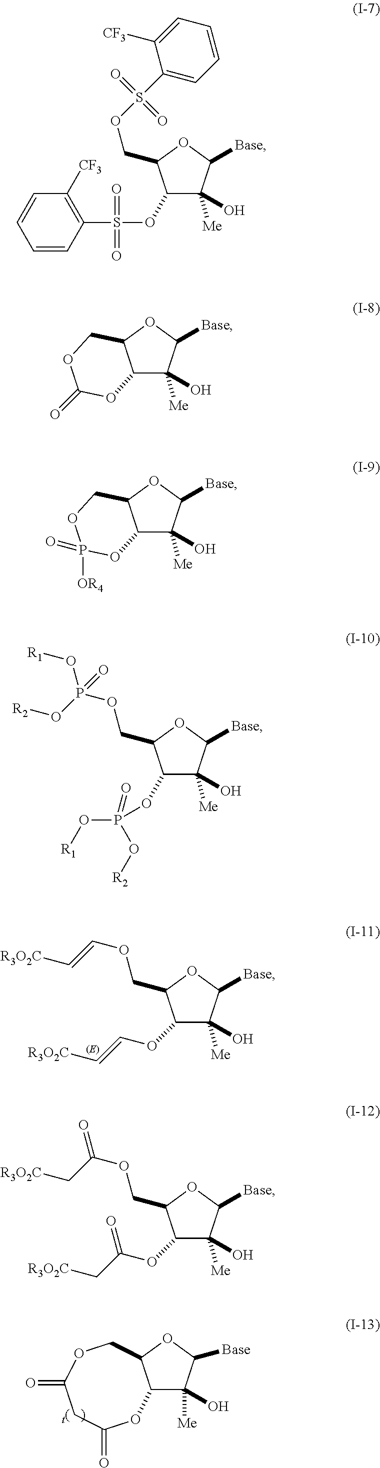

wherein in the formulae Base, R₁, R₂, R₃, R₄ and t are as defined above.

More preferred compounds of formula (I) are compounds having any of formulae (I-1), (I-2), (I-3) and (I-4)

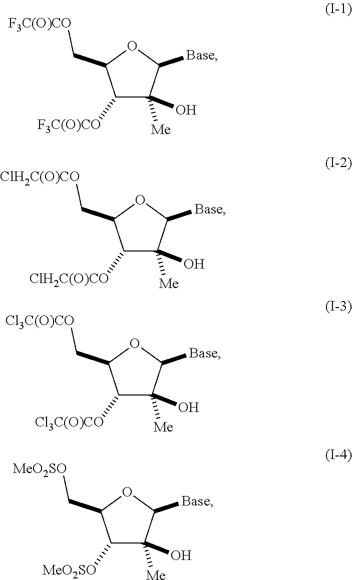

wherein Base is as defined for formula (I).

More preferred compounds of formula (I) are compounds having any of formulae (I-1′) to (I-13′)

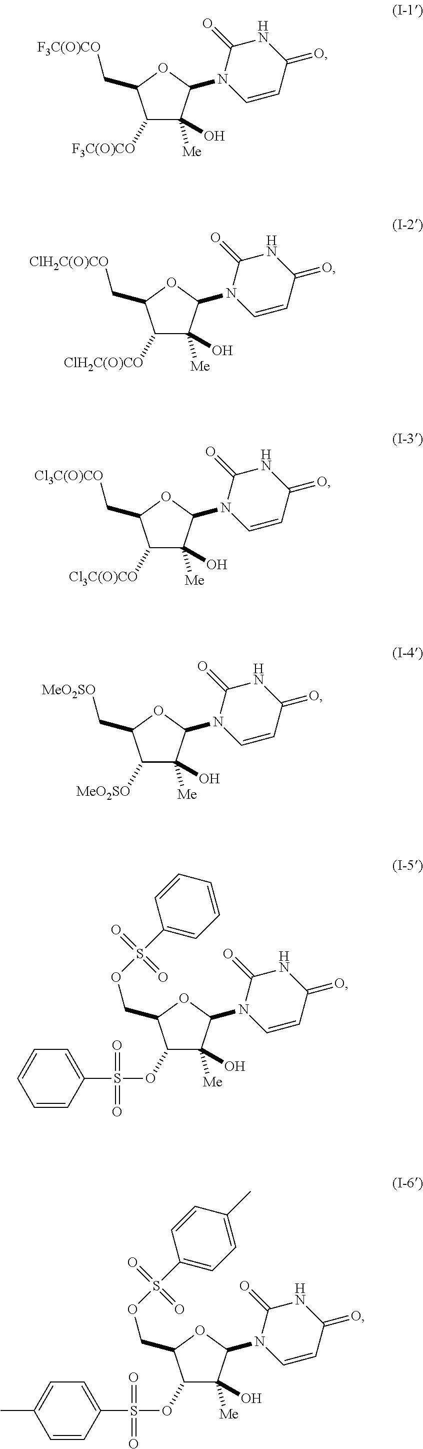

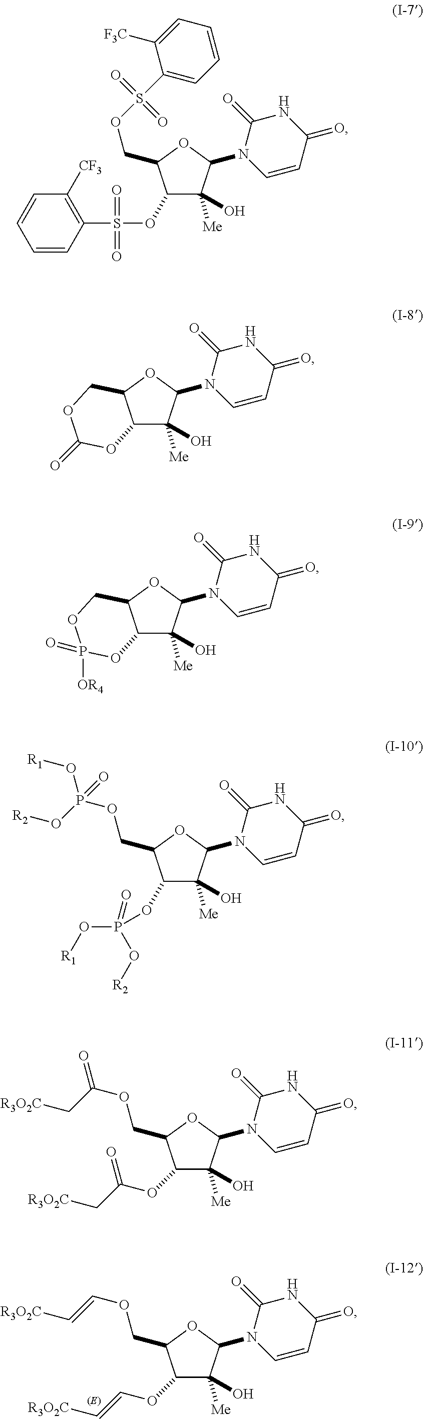

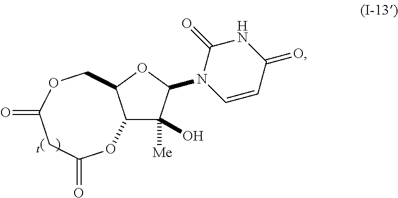

wherein in the formulae Base, R₁, R₂, R₃, R₄ and t are as defined above.

More preferred compounds of formula (I) are compounds having any of formulae (I-1′), (I-2′), (I-3′) and (I-4′)

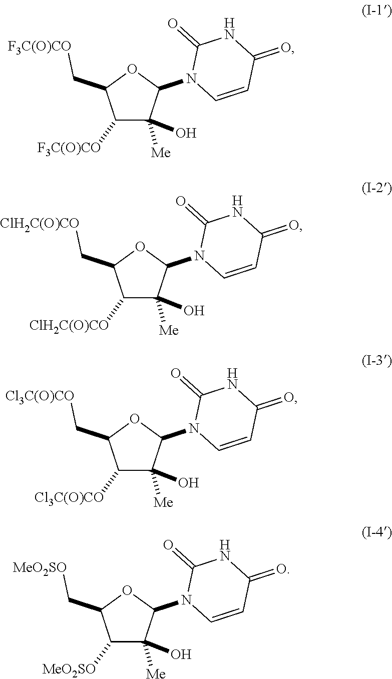

Further, it is provided a compound of formula (I) or isomers, stereoisomers, diastereomers, enantiomers or salts thereof, preferably a compound of formula (I), obtained or obtainable by any of the processes as disclosed above. Preferably, the compound of formula (I) is a compound of any of formulae (I-1), (I-2), (I-3), (I-4), (I-5), (I-6), (I-7), (I-8), (I-9), (I-10), (I-11), (I-12), (I-13), (I-1′), (I-2′), (I-3′), (I-4′) (I-5′), (I-6′), (I-7′), (I-8′), (I-9′), (I-10′), (I-11′), (I-12′), (I-13′), wherein all the formulae are as disclosed above; more preferably, the compound of formula (I) is a compound of any of formulae (I-1), (I-2), (I-3), (I-4) (I-1′), (I-2′), (I-3′) and (I-4′) wherein all the formulae are as disclosed above, even more preferably the compound of formula (I) is a compound of formula (I-2) or (I-3).

Further, it is provided a mixture comprising a compound of formula (I) or isomers, stereoisomers, diastereomers, enantiomers or salts thereof obtained or obtainable by any of the processes as disclosed above. Preferably, the compound of formula (I) is a compound of any of formulae (I-1), (I-2), (I-3), (I-4), (I-5), (I-6), (I-7), (I-8), (I-9), (I-10), (I-11), (I-12), (I-13), (I-1′), (I-2′), (I-3′), (I-4′) (I-5′), (I-6′), (I-7′), (I-8′), (I-9′), (I-10′), (I-11′), (I-12′), (I-13′), wherein all the formulae are as disclosed above. More preferably, the compound of formula (I) comprised in the mixture is a compound of any of formulae (I-1), (I-2), (I-3), (I-4), (I-1′), (I-2′), (I-3′) and (I-4′) wherein all the formulae are as disclosed above; even more preferably the compound of formula (I) is a compound of formula (I-2′) or (I-3′).

Compounds of formula (I) are used as intermediates in the preparation of compounds of formula (II). Advantageously, the use of the compounds of formula (I) in the fluorination process as provided herein leads to the corresponding fluorinated compounds of formulae (II) and (III) in a high yield. Compounds of formula (III) are intermediates in the synthesis of nucleoside phosphoramidates. Therefore the use of compounds of formula (I) for preparing compounds of formula (III) results in a high yield and hence in an efficient and economic process for the preparation of nucleoside phosphoramidates such as sofosbuvir.

### Compound of Formula (II)

Yet further, it is provided a compound of formula (II) or isomers, stereoisomers, diastereomers, enantiomers or salts thereof, preferably the compound of formula (II)

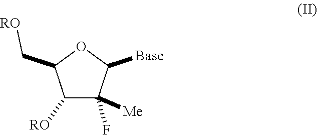

wherein radicals R and Base are as defined above for compounds of formula (I).

Preferred compounds of formula (II) are compounds having any of formulae (II-1) to (I-13)

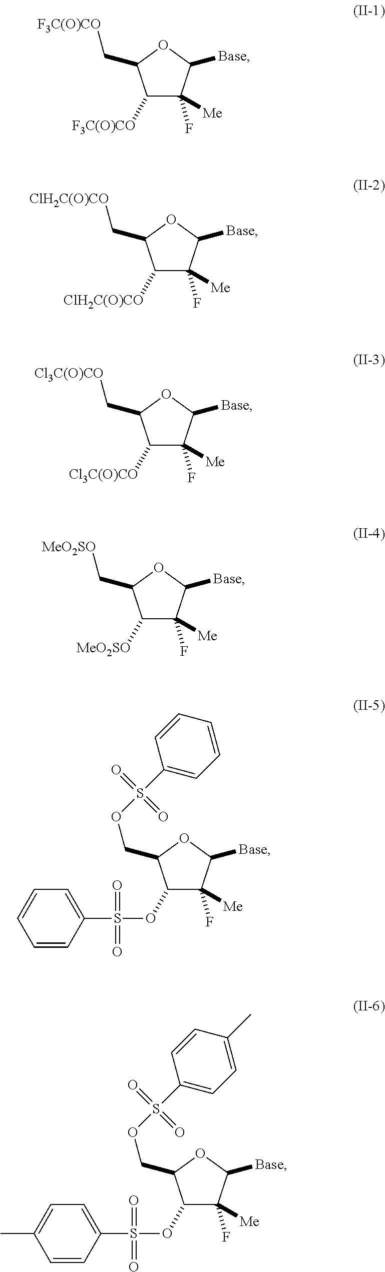

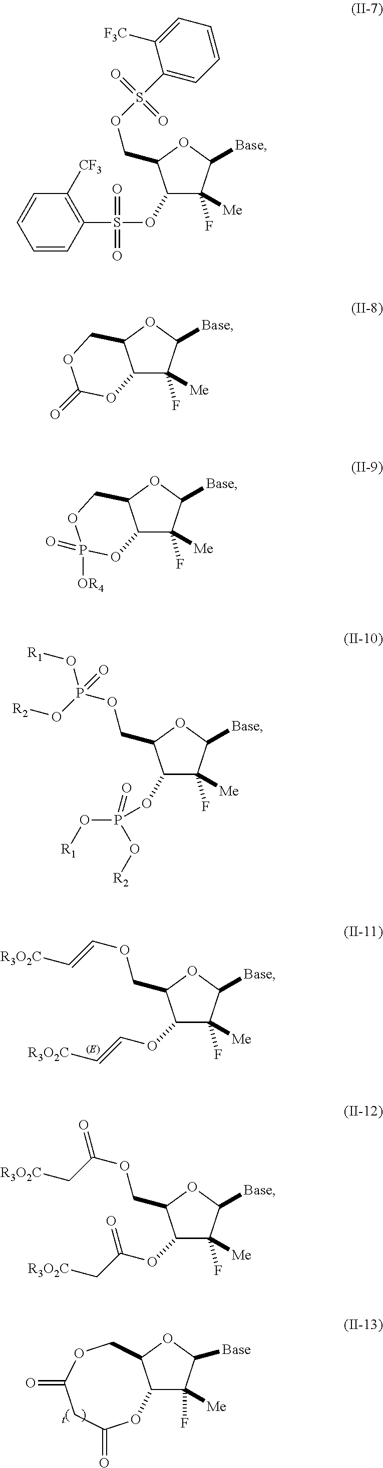

wherein in the formulae Base, R₁, R₂, R₃, R₄ and t are as defined above.

Preferred compounds of formula (II) are compounds having any of formulae (II-1), (II-2), (II-3) and (II-4)

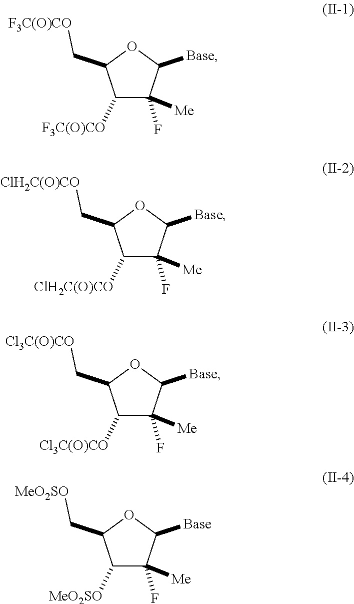

wherein Base is as defined for formulae (I) and (II).

More preferably, the present invention relates to compound of formula (II) having any of formula (II-1′) to (II-13′)

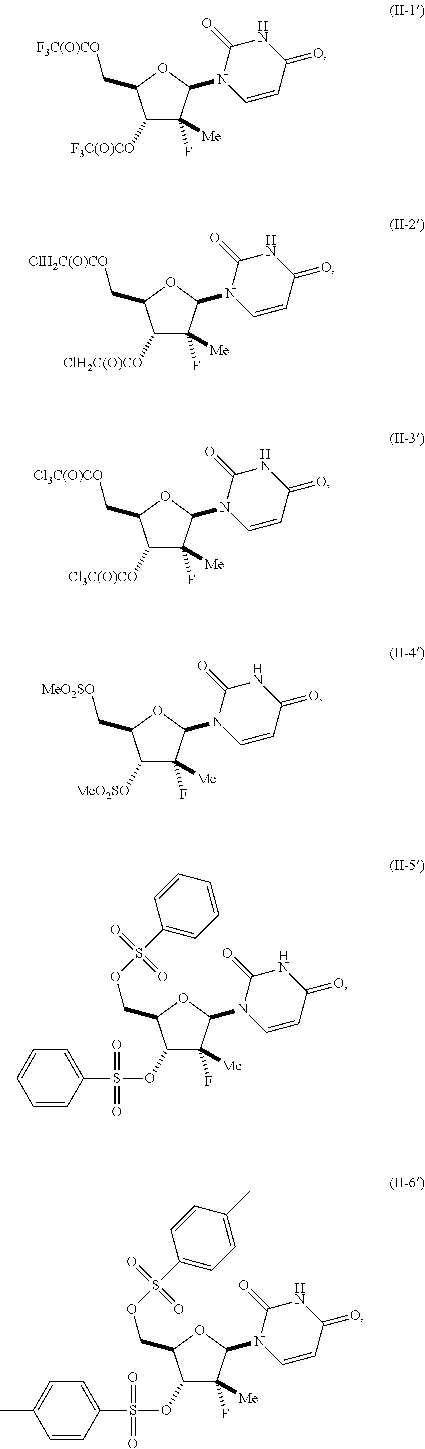

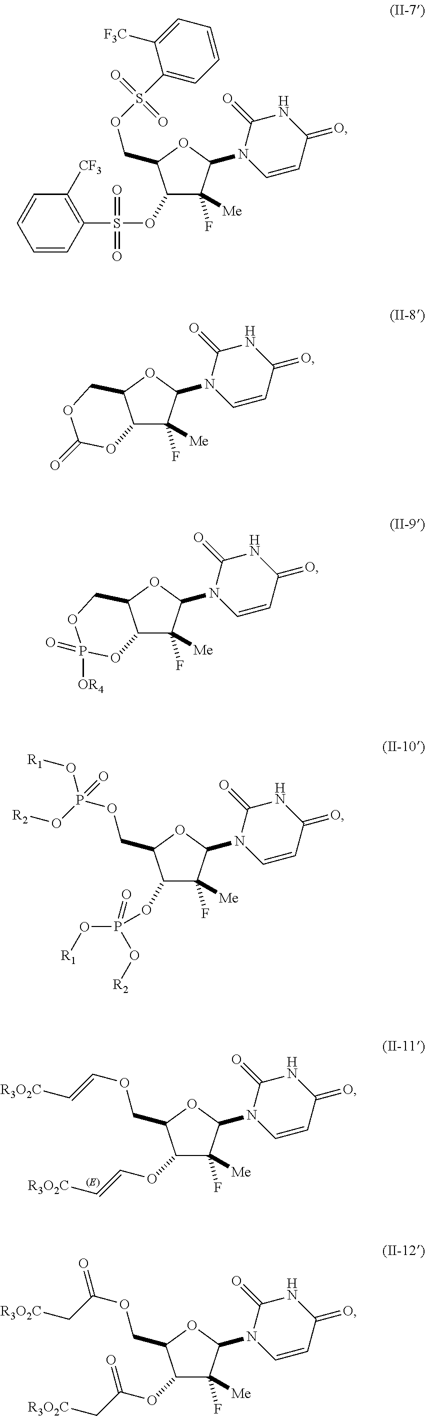

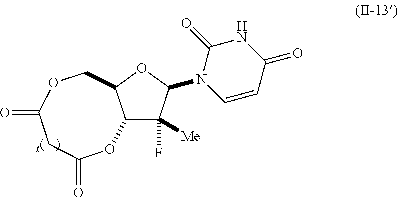

wherein in the formulae Base, R₁, R₂, R₃, R₄ and t are as defined above.

More preferred compounds of formula (II) are compounds of any of formulae (II-1′), (II-2′) or (II-3′) and (II-4′)

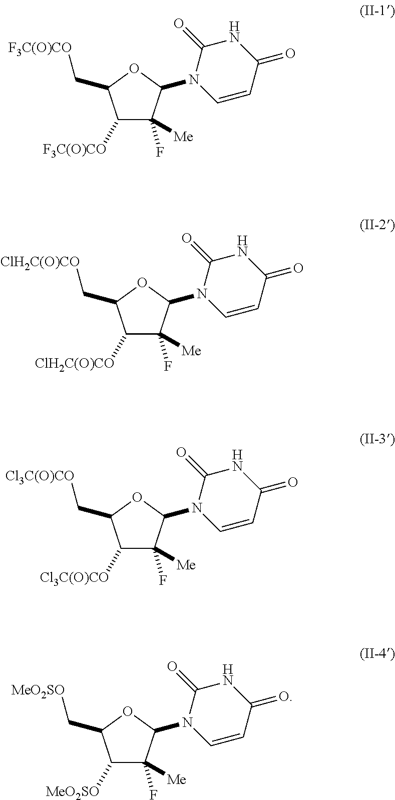

Further the present invention provides a compound of formula (II) or isomers, stereroisomers, diastereomers, enantiomers or salts thereof, obtained or obtainable by any of the processes as disclosed above. Preferably, the compound of formula (II) is a compound of any of formulae (II-1), (II-2), (II-3), (II-4), (II-5), (II-6), (II-7), (II-8), (II-9), (II-10), (II-11), (II-12), (II-13), (II-1′), (II-2′), (II-3′), (II-4′) (II-5′), (II-6′), (II-7′), (II-8′), (II-9′), (II-10′), (II-11′), (II-12′), (II-13′) wherein all the formulae are as disclosed above. More preferably, the compound of formula (II) is a compound of any of formulae (II-1), (II-2), (II-3), (II-4), (II-1′), (II-2′), (II-3′) and (II-4′) wherein all the formulae are as disclosed above. Even more preferably, the compound of formula (II) is a compound of formula (II-2) or (II-3).

Further, it is provided a mixture comprising a compound of formula (II) or isomers, stereoisomers, diastereomers, enantiomers or salts thereof obtained or obtainable by any of the processes as disclosed above. Preferably, the compound of formula (II) comprised in the mixture is a compound of any of formulae (II-1), (II-2), (II-3), (II-4), (II-5), (II-6), (II-7), (II-8), (II-9), (II-10), (II-11), (II-12), (II-13), (II-1′), (II-2′), (II-3′), (II-4′) (II-5′), (II-6′), (II-7′), (II-8′), (II-9′), (II-10′), (II-11′), (II-12′), (II-13′) wherein all the formulae are as disclosed above. More preferably, the compound of formula (II) comprised in the mixture is a compound of any of formulae (II-1), (II-2), (II-3), (II-4), (II-1′), (II-2′), (II-3′) and (II-4′) wherein all the formulae are as disclosed above.

Use of the Compound of Formula (I) as a Reagent for Preparing Nucleoside Phosphoramidate

Yet further, it is provided the use of a compound of formula (I) or isomers, stereoisomers, diastereomers, enantiomers or salts thereof as disclosed above, as a reagent in the preparation of a 2′ fluoro-nucleoside-phosphoramidate. Preferably, the compound of formula (I) is a compound of any of formulae (I-1), (I-2), (I-3), (I-4), (I-5), (I-6), (I-7), (I-8), (I-9), (I-10), (I-11), (I-12), (I-13), (I-1′), (I-2′), (I-3′), (I-4′) (I-5′), (I-6′), (I-7′), (I-8′), (I-9′), (I-10′), (I-11′), (I-12′), (I-13′), wherein all the formulae are as disclosed above. More preferably, the compound of formula (I) is a compound of any of formulae (I-1), (I-2), (I-3), (I-4), (I-1′), (I-2′), (I-3′), (I-4′), wherein all the formulae are as disclosed above, as a reagent in the preparation of a 2′ fluoro-nucleoside-phosphoramidate. Even more preferably, the compound of formula (I) is a compound of any of formulae (I-2), (I-3), (I-2′) and (I-3′). 2′ fluoro-nucleoside phosphoramidates are nucleoside prodrug compounds. The preparation of phosphoramidates as prodrug of nucleosides, in general, and of 2′ fluoro-nucleoside, in particular, is disclosed for example in patent application WO2008/121634 and in J. Org. Chem. 2011, 76, 8311 and Bioorg. Med. Chem. 2012, 20, pp. 4801.

The compound of formula (I) or isomers, stereoisomers, diastereomers, enantiomers or salts thereof, as disclosed above is preferably used as a reagent in the preparation of a 2′ fluoro-nucleoside phosphoramidates of formula (X)

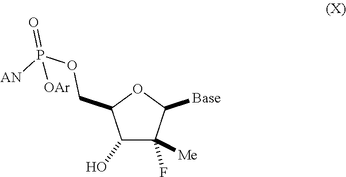

wherein

Ar is an optionally substituted aryl, preferably phenyl or naphtyl;

AN is an aryl ester or a C₁-C₅ alkyl ester of an amino acid, preferably of a natural amino acid, wherein preferably the natural amino acid is alanine; preferably, the ester is an isopropyl ester; and Base is as defined above in formula (I).

The phosphorous is a chiral atom having chirality (Sp) or (Rp). Hence, the compound of formula (X) can be a single diastereoisomer (Sp) or (Rp) or a diastereoisomer mixture thereof.

Preferably, the compound of formula (I) is a compound of any of formulae (I-1), (I-2), (I-3), (I-4), (I-5), (I-6), (I-7), (I-8), (I-9), (I-10), (I-11), (I-12), (I-13), (I-1′), (I-2′), (I-3′), (I-4′) (I-5′), (I-6′), (I-7′), (I-8′), (I-9′), (I-10′), (I-11′), (I-12′), (I-13′), wherein all the formulae are as disclosed above. More preferably, the compound of formula (I) is a compound of any of formulae (I-1), (I-2), (I-3), (I-4), (I-1′), (I-2′), (I-3′), (I-4′) wherein all the formulae are as disclosed above. Even more preferably, the compound of formula (I) is a compound of any of formulae (I-2), (I-3), (I-2′) and (I-3′).

Preferably, the 2′ fluoro-nucleoside phosphoramidate compound of formula (X) is sofosbuvir of the formula (X′)

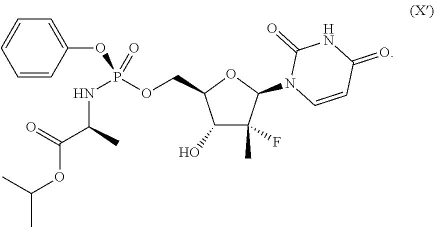 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

As already mentioned above, compounds of formula (I) are used as intermediates in the preparation of compounds of formula (II). Advantageously, the use of the compounds of formula (I) in the fluorination process as provided herein leads to the corresponding fluorinated compounds of formula (II) in a high yield. This ultimately results in a high yield and hence in an efficient and economic process for the preparation of nucleoside phosphoramidates such as sofosbuvir.

Use of the Compound of Formula (II) as a Reagent for Preparing Nucleoside Phosphoramidate

Yet further, it is provided the use of a compound of formula (II) as disclosed above or isomers, stereoisomers, diastereomers, enantiomers or salts thereof, as disclosed above, as a reagent in the preparation of a 2′ fluoro-nucleoside phosphoramidate, preferably of a 2′ fluoro-nucleoside phosphoramidate of formula (X) as disclosed above, more preferably of a 2′ fluoro-nucleoside phosphoramidate of formula (X′)

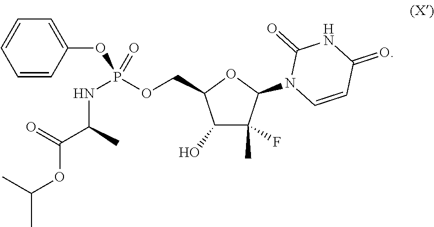 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

Preferably, the compound of formula (II) is a compound of any of formulae (II-1), (II-2), (II-3), (II-4), (II-5), (II-6), (II-7), (II-8), (II-9), (II-10), (II-11), (II-12), (II-13), (II-1′), (II-2′), (II-3′), (II-4′) (II-5′), (II-6′), (II-7′), (II-8′), (II-9′), (II-10′), (II-11′), (II-12′), (II-13′) wherein all the formulae are as disclosed above. Preferably, the compound of formula (II) is a compound of any formulae (II-1), (II-2), (II-3), (II-4), (II-1′), (II-2′), (II-3′), (II-4′) all as disclosed above.

Preferably, the compound of formula (II) is comprised in a mixture obtainable or obtained by any of the processes as disclosed above.

Yet further, the present invention is directed to a process for the preparation of sofosbuvir of formula (X′)

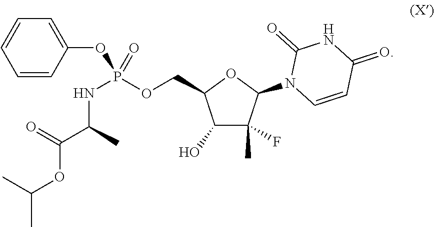 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

The process comprises

(x) reacting a compound of formula (II) and obtaining a compound of formula (III); and

(xx) reacting the compound of formula (III) and obtaining sofosbuvir, wherein the compounds of formulae (II) and (III) are as defined above.

As to the reacting of (x), no limitation exists as to the carry out of this reaction provided that a compound of formula (III) is obtained from a compound of formula (II). It is preferred that the reacting of (x) is carried out by any of the processes as disclosed above.

As to the reacting of (xx), no limitation exists as to the carry out of this reaction provided that a compound of formula (X′) is obtained from a compound of formula (III). By way of a non limiting example, the reacting of (xx) can be carried out according to a process disclosed in patent application WO 2008/121634 and in J. Org. Chem. 2011, 76, 8311 and Bioorg. Med. Chem. 2012, 20, 4801.

Yet further, it is provided a process for the preparation of sofosbuvir of formula (X′)

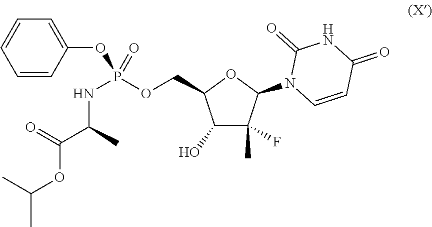 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

comprising

(xx′) reacting the compound of formula (III′)

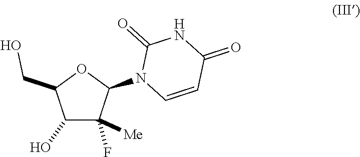 [SMILES: C[C@@]1(F)[C@H](O)[C@@H](CO)O[C@H]1n1ccc(=O)[nH]c1=O]

to sofosbuvir.

It is preferred that the compound of formula (III′) is prepared by any of the processes as disclosed above.

Mixture Comprising a Compound of Formula (II) or (III)

Further, it is provided a mixture comprising a compound of formula (II) or isomers, stereoisomers, diastereomers, enantiomers or salts thereof

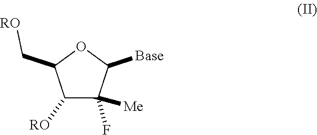

wherein the radicals R and Base are as defined above in formula (I),

wherein the mixture preferably comprises a compound of any of formulae (II-1), (II-2), (II-3), (II-4), (II-5), (II-6), (II-7), (II-8), (II-9), (II-10), (II-11), (II-12), (II-13), (II-1′), (II-2′), (II-3′), (II-4′) (II-5′), (II-6′), (II-7′), (II-8′), (II-9′), (II-10′), (II-11′), (II-12′), (II-13′) wherein all the formulae are as disclosed above; more preferably the mixture comprises a compound of any of formulae (II-1), (II-2), (II-3) and (II-4)

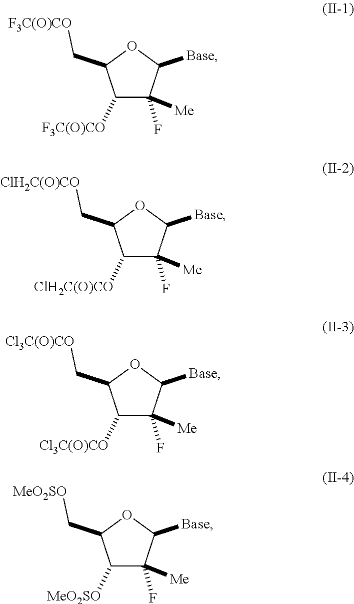

wherein Base is as defined for formulae (I) and (II),

wherein more preferably the mixture comprises a compound of any of formulae (II-1′) or (II-2) or (II-3′) or (II-4′)

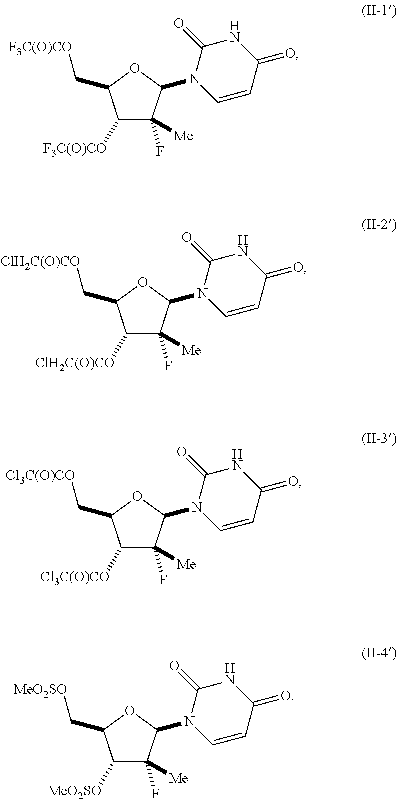

Preferably, the mixture, as defined above, comprising a compound of formula (II) has a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

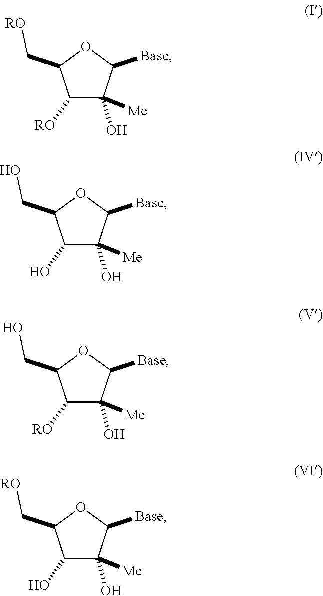

wherein at each occurrence

R is an inert electron withdrawing OH protecting group; and

Base is a purinyl residue or a pyrimidinyl residue linked to the furanose ring according to formulae (II), (I′), (IV′), (V′) and (VI′) through a carbon or a nitrogen atom,

wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).

It is preferred that the mixture comprising the compound of formula (II) has a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof, wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′); and

wherein in the formulae at each occurrence R is preferably selected from the group consisting of X₃₋ₙHₙCC(O) wherein X is halogen, preferably Cl or F, more preferably Cl and n is 0, 1, or 2; or R is preferably selected from the group consisting of SO₂Me, SO₂-p-Me-Ph (tosyl), SO₂-p-NO₂-Ph (para-nosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me, or

R is preferably selected from SO₂Ph or SO₂-o-CF₃-Ph (ortho-trifluoromethylphenyl); or

R is preferably

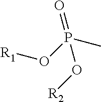

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or

R is preferably selected from CH═CH₂—CO₂R₃ or C(O)—CH₂—CO₂R₃ wherein R3 is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group selected from C(O), C(O)—(CH₂)ₜ—CO or

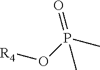

wherein R₄ is selected from the group consisting of alkyl, aryl and cycloalkyl and

wherein t is 1 or 2;

Regarding R₁, when R₁ is alkyl the alkyl is preferably C₁-C₆ alkyl, more preferably C₁-C₄ alkyl, even more preferably C₁-C₂ alkyl; when R₁ is an aryl, aryl is preferably selected from phenyl or naphtyl, more preferably is phenyl.

Regarding R₂, when R₂ is alkyl, the alkyl is preferably is C₁-C₆ alkyl, more preferably C₁-C₄ alkyl, even more preferably C₁-C₂ alkyl, when R₂ is cycloalkyl, cycloalkyl is preferably a C₃-C₆ cycloalkyl, more preferably is a C₅-C₆ cycloalkyl; when R₂ is an aryl, aryl is preferably selected from phenyl or naphtyl, more preferably phenyl.

Regarding R₃, when R₃ is alkyl, the alkyl is preferably is C₁-C₆ alkyl, more preferably C₁-C₄ alkyl, even more preferably C₁-C₂ alkyl, when R₃ is cycloalkyl, cycloalkyl is preferably a C₃-C₆ cycloalkyl, more preferably is a C₅-C₆ cycloalkyl; when R₃ is an aryl, aryl is preferably selected from phenyl or naphtyl, more preferably phenyl.

Regarding R₄, when R₄ is alkyl, the alkyl is preferably is C₁-C₆ alkyl, more preferably C₁-C₄ alkyl, even more preferably C₁-C₂ alkyl, when R₄ is cycloalkyl, cycloalkyl is preferably a C₃-C₆ cycloalkyl, more preferably is a C₅-C₆ cycloalkyl; when R₄ is an aryl, aryl is preferably selected from phenyl or naphtyl, more preferably phenyl.

Regarding q, q is preferably selected from 2, 3 and 4.

R is more preferably selected from the group consisting of X₃₋ₙHₙCC(O) wherein X is halogen, preferably Cl or F, more preferably Cl and n is 0, 1, or 2; or R is preferably selected from the group consisting of SO₂Me, SO₂-p-Me-Ph (tosyl), SO₂-p-NO₂-Ph (para-nosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me. Even more preferably R is selected from Cl₃CC(O) or Cl₂HCC(O).

Regarding the radical Base, Base is a purinyl residue or a pyrimidinyl residue linked to the furanose ring according to formulae (II), (I′), (IV′), (V′) and (VI′) through a carbon or nitrogen atom, preferably, Base is selected from the group consisting of uridine, protected uridine, thymine, protected thymine, cytidine, protected cytidine, adenosine, protected adenosine, guanine, protected guanine. More preferably Base is selected from the group consisting of uridine, thymine, cytidine, adenosine, guanine; more preferably Base is uridine.

Yet further, it is provided a mixture comprising a compound of formula (II) obtainable or obtained by any of the processes disclosed above, preferably by the reaction of (ii), having a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

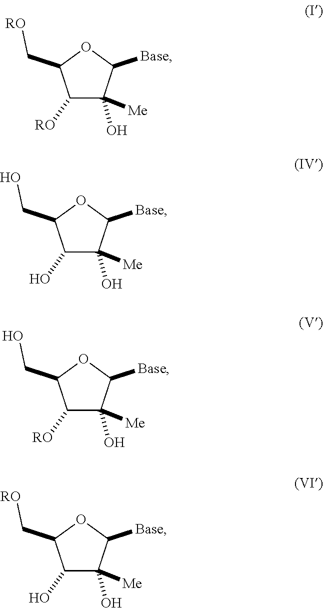

wherein at each occurrence R is an inert electron withdrawing OH protecting group; and Base is a purinyl residue or a pyrimidinyl residue linked to the furanose ring according to formulae (I), (II) and (III) through a carbon or nitrogen atom, wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).

It is more preferred that the mixture obtained or obtainable by any of the processes as disclosed above, preferably by the reaction of (ii), comprising the compound of formula (II) has a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof, wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′); and wherein R is preferably selected from the group consisting of X₃₋ₙHₙCC(O) wherein X is halogen preferably Cl or F, more preferably Cl and n is 0, 1, or 2; or R is preferably selected from the group consisting of SO₂Me, SO₂-p-Me-Ph (tosyl), SO₂-p-NO₂-Ph (paranosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me or R is preferably selected from SO₂Ph or SO₂-o-CF₃-Ph (ortho-trifluoromethylphenyl); or

R is preferably

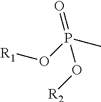

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or

R is preferably selected from CH═CH₂—CO₂R₃ or C(O)—CH₂—CO₂R₃ wherein R₃ is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group

selected from C(O), C(O)—(CH₂)ₜ—CO or

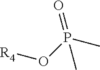

wherein R₄ is selected from the group consisting of alkyl, aryl and cycloalkyl and

wherein t is 1 or 2.

Regarding R₁, when R₁ is alkyl, the alkyl is preferably C₁-C₆ alkyl, more preferably C₁-C₄ alkyl, even more preferably C₁-C₂ alkyl; when R₁ is an aryl, aryl is preferably selected from phenyl or naphtyl, more preferably is phenyl.

Regarding R₂, when R₂ is alkyl, the alkyl is preferably is C₁-C₆ alkyl, more preferably C₁-C₄ alkyl, even more preferably C₁-C₂ alkyl, when R₂ is cycloalkyl, cycloalkyl is preferably a C₃-C₆ cycloalkyl, more preferably is a C₅-C₆ cycloalkyl; when R₂ is an aryl, aryl is preferably selected from phenyl or naphtyl, more preferably phenyl

Regarding R₃, when R₃ is alkyl, the alkyl is preferably is C₁-C₆ alkyl, more preferably C₁-C₄ alkyl, even more preferably C₁-C₂ alkyl, when R₃ is cycloalkyl, cycloalkyl is preferably a C₃-C₆ cycloalkyl, more preferably is a C₅-C₆ cycloalkyl; when R₃ is an aryl, aryl is preferably selected from phenyl or naphtyl, more preferably phenyl.

Regarding R₄, when R₄ is alkyl, the alkyl is preferably is C₁-C₆ alkyl, more preferably C₁-C₄ alkyl, even more preferably C₁-C₂ alkyl, when R₄ is cycloalkyl, cycloalkyl is preferably a C₃-C₆ cycloalkyl, more preferably is a C₅-C₆ cycloalkyl; when R₄ is an aryl, aryl is preferably selected from phenyl or naphtyl, more preferably phenyl.

Regarding q, q is preferably selected from 2, 3 and 4.

R is more preferably selected from the group consisting of X₃₋ₙHₙCC(O) wherein X is halogen, preferably Cl or F, more preferably Cl and n is 0, 1, or 2; or R is preferably selected from the group consisting of SO₂Me, SO₂-p-Me-Ph (tosyl), SO₂-p-NO₂-Ph (para-nosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me. Even more preferably, R is selected from Cl₃CC(O) or Cl₂HCC(O).

Regarding the radical Base, Base is a purinyl residue or a pyrimidinyl residue linked to the furanose ring according to formulae (II), (I′), (IV′), (V′) and (VI′) through a carbon or nitrogen atom, preferably, Base is selected from the group consisting of uridine, protected uridine, thymine, protected thymine, cytidine, protected cytidine, adenosine, protected adenosine, guanine, protected guanine, more preferably Base is selected from the group consisting of uridine, thymine, cytidine, adenosine, guanine; more preferably Base is uridine.

Hence, with respect to the prior art patent application WO 2005/003147 A wherein DAST is used as fluorinating agent, the present invention provides the advantage that no 2′ epimer of the starting non-fluorinated nucleoside is formed. On the contrary, in the fluorination process carried out with DAST by products are formed that need to be separated chromatographically, namely the 2′ epimer of the starting non-fluorinated nucleoside and the elimination product are formed such as compounds B and C exemplarily shown below in which the protecting group used is Bz.

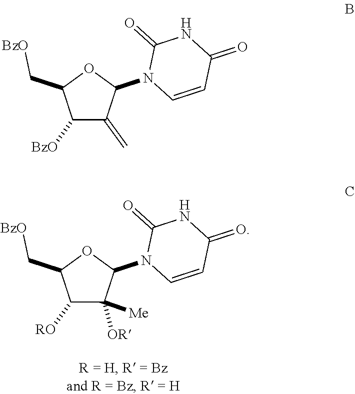

Yet, it is provided a mixture comprising a compound of formula (III) or isomers, stereoisomers, diastereomers, enantiomers or salts thereof obtainable or obtained by any of the processes as disclosed above, preferably by the reaction of (iii), having a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

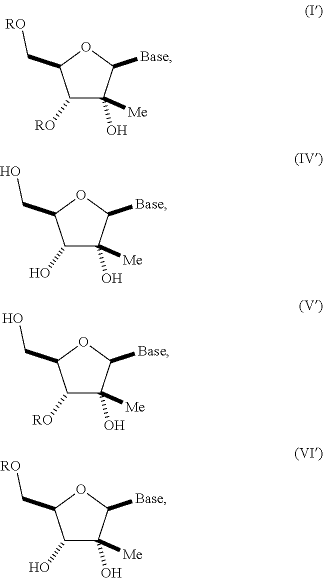

wherein at each occurrence,

R is an inert electron withdrawing OH protecting group; and

Base is a purinyl residue or a pyrimidinyl residue linked to the furanose ring according to formulae (III), (I′), (IV′), (V′) and (VI′) through a carbon or nitrogen atom,

wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).

It is more preferred that the mixture obtained or obtainable by any of the processes as disclosed above, preferably by the reaction of (iii), comprising the compound of formula (III) has a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof,

wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′); and wherein R is preferably selected from the group consisting of X₃₋ₙHₙCC(O) wherein X is halogen preferably Cl or F, more preferably Cl and n is 0, 1, or 2; or R is preferably selected from the group consisting of SO₂Me, SO₂-p-Me-Ph (tosyl), SO₂-p-NO₂-Ph (para-nosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me or R is preferably selected from SO₂Ph or SO₂-o-CF₃-Ph (ortho-trifluoromethylphenyl); or

R is preferably

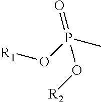

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or

R is preferably selected from CH═CH₂—CO₂R₃ or C(O)—CH₂—CO₂R₃ wherein R₃ is selected from the group consisting of alkyl, aryl and cycloalkyl; or

wherein the radical R attached to the oxygen in position 5′ of the sugar moiety taken together with the radical R attached to the oxygen in position 3′ of the sugar moiety forms a group

selected from C(O), C(O)—(CH₂)ₜ—CO or

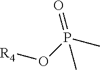

wherein R₄ is selected from the group consisting of alkyl, aryl and cycloalkyl and

wherein t is 1 or 2.

R is more preferably selected from the group consisting of X₃₋ₙHₙCC(O) wherein X is halogen preferably Cl or F, more preferably Cl and n is 0, 1, or 2; or R is preferably selected from the group consisting of SO₂Me, SO₂-p-Me-Ph (tosyl), SO₂-p-NO₂-Ph (para-nosyl), SO₂-o-NO₂-Ph (ortho-nosyl), SO₂CF₃ (triflyl), more preferably wherein R is selected from the group consisting of F₃CC(O), Cl₃CC(O), ClH₂CC(O), Cl₂HCC(O), F₂HCC(O), FH₂CC(O) and SO₂Me Even more preferably, R is selected from Cl₃CC(O) or Cl₂HCC(O).

Regarding the radical Base, Base is a purinyl residue or a pyrimidinyl residue linked to the furanose ring according to formulae (II), (I′), (IV′), (V′) and (VI′) through a carbon or nitrogen atom, preferably, Base is selected from the group consisting of uridine, protected uridine, thymine, protected thymine, cytidine, protected cytidine, adenosine, protected adenosine, guanine, protected guanine, more preferably Base is selected from the group consisting of uridine, thymine, cytidine, adenosine, guanine; more preferably Base is uridine.

The present invention is further illustrated by the following embodiments and combinations of embodiments as indicated by the respective dependencies and references.

Process for Preparing a Compound of Formula (II) or (III)
1. A process for the preparation of a compound of formula (II)

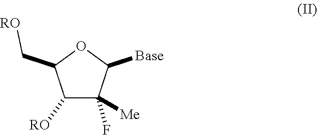

or of a compound of formula (III)

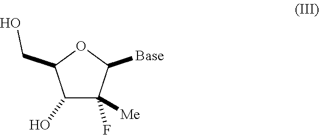

comprising
(i) providing a mixture comprising a compound of formula (I) or isomers, stereoisomers, diastereomers, enantiomers or salts thereof

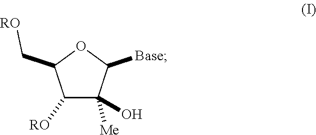

(ii) subjecting the mixture provided in (i) to fluorinating conditions in the presence of a fluorination agent selected from the group consisting of diethylamino(difluoro)sulfoniumtetrafluoroborate and difluoro(morpholino)sulfonium tetrafluoroborate obtaining a mixture comprising a compound of formula (II) or isomers, stereoisomers, diastereomers, enantiomers or salts thereof

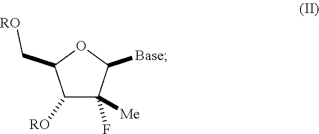

(iii) optionally subjecting the mixture obtained in (ii) to deprotection conditions, obtaining a mixture comprising the compound of formula (III) or isomers, stereoisomers, diastereomers, enantiomers, or salts thereof

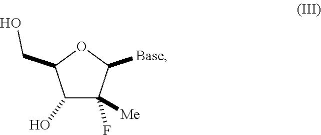

wherein at each occurrence
R is an inert electron withdrawing OH protecting group; and
Base is a purinyl residue or a pyrimidinyl residue linked to the furanose ring according to formulae (I), (II) and (III) through a carbon or a nitrogen atom.
2. A process for the preparation of a compound of formula (II)

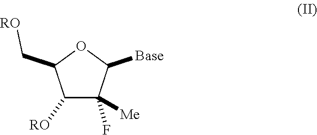

or of a compound of formula (III)

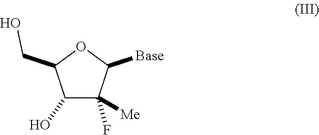

comprising
(i) providing a mixture comprising a compound of formula (I) or isomers, diastereomers, enantiomers or salts thereof

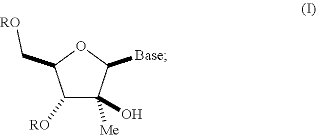

(ii) subjecting the mixture provided in (i) to fluorinating conditions in the presence of a fluorination agent selected from the group consisting of diethylamino(difluoro)sulfonium tetrafluoroborate and difluoro(morpholino)sulfonium tetrafluoroborate obtaining a mixture comprising a compound of formula (II) or isomers, diastereomers, enantiomers or salts thereof

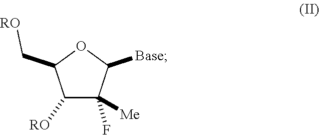

(iii) optionally subjecting the mixture obtained in (ii) to deprotection conditions, obtaining a mixture comprising the compound of formula (III) or isomers, diastereomers, enantiomers, or salts thereof

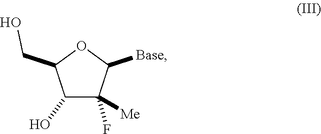

wherein at each occurrence
R is an inert electron withdrawing OH protecting group; and
Base is a purinyl residue or a pyrimidinyl residue linked to the furanose ring according to formulae (I), (II) and (III) through a carbon or a nitrogen atom.
3. The process of embodiment 1 or 2, wherein
   R is selected from the group consisting of X₃₋ₙHₙCC(O) wherein X is halogen and n is 0, 1, or 2; or R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).
4. The process of embodiment 1 or 2, wherein
   R is selected from SO₂Ph or SO₂-o-CF₃-Ph (ortho-trifluoromethylphenyl); or
   R is

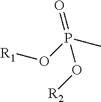

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or
R is selected from CH═CH₂—CO₂R₃ or C(O)—CH₂—CO₂R₃ wherein R₃ is selected from the group consisting of alkyl, aryl and cycloalkyl; or
wherein the radical R attached to the oxygen in position 5′ of the sugar moiety taken together with the radical R attached to the oxygen in position 3′ of the sugar moiety forms a group selected from C(O), C(O)—(CH₂)ₜ—CO or

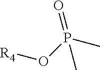

and
wherein R₄ is selected from the group consisting of alkyl, aryl and cycloalkyl and wherein t is 1 or 2.
5. The process of embodiment 4, wherein R₁ is C₁-C₆ alkyl, preferably C₁-C₄ alkyl, more preferably C₁-C₂ alkyl, or R₁ is an aryl selected from phenyl or naphtyl.
6. The process of embodiment 4 or 5, wherein R₂ is C₁-C₆ alkyl, preferably C₁-C₄ alkyl, more preferably C₁-C₂ alkyl, or R₂ is a C₃-C₆ cycloalkyl, preferably is a C₅-C₆ cycloalkyl or R₂ is an aryl selected from phenyl or naphtyl.
7. The process of embodiment 4, wherein R₃ is C₁-C₆ alkyl, preferably C₁-C₄ alkyl, more preferably C₁-C₂ alkyl, or R₃ is a C₃-C₆ cycloalkyl, preferably is a C₅-C₆ cycloalkyl or R₃ is an aryl selected from phenyl or naphtyl.
8. The process of embodiment 4, wherein R₄ is C₁-C₆ alkyl, preferably C₁-C₄ alkyl, more preferably C₁-C₂ alkyl, or R₄ is a C₃-C₆ cycloalkyl, preferably is a C₅-C₆ cycloalkyl or R₄ is an aryl selected from phenyl or naphtyl.
9. The process of any of embodiments 4 to 6, wherein q is selected from 2, 3 and 4.
10. The process of any of embodiments 1 to 3, wherein R is selected from the group consisting of F₃CC(O), Cl₃CC(O), ClH₂CC(O), Cl₂HCC(O), F₂HCC(O), FH₂CC(O) and SO₂Me.
11. The process of any of embodiments 1 to 10, wherein Base is selected from the group consisting of uridine, protected uridine, thymine, protected thymine, cytidine, protected cytidine, adenosine, protected adenosine, guanine and protected guanine.
12. The process of any of embodiments 1 to 11, wherein Base is uridine.
13. The process of any of embodiments 1, 2, 3, 10, 11, 12, wherein the compound of formula (I) is a compound of formula (I-1) or (I-2) or (I-3) or (I-4)

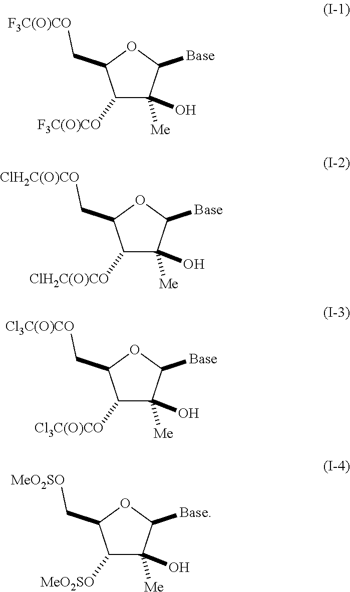

14. The process of any of embodiments 1 to 12, wherein the compound of formula (I) is a compound of any of formulae (I-5) to (I-13)

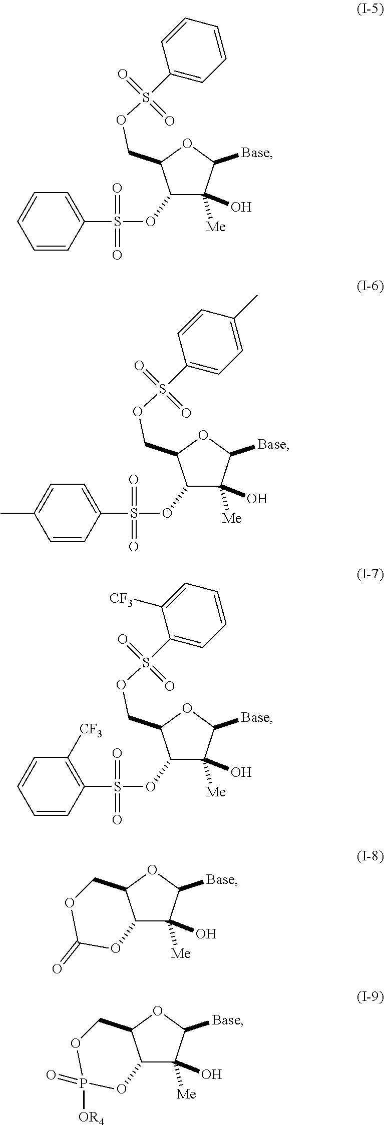

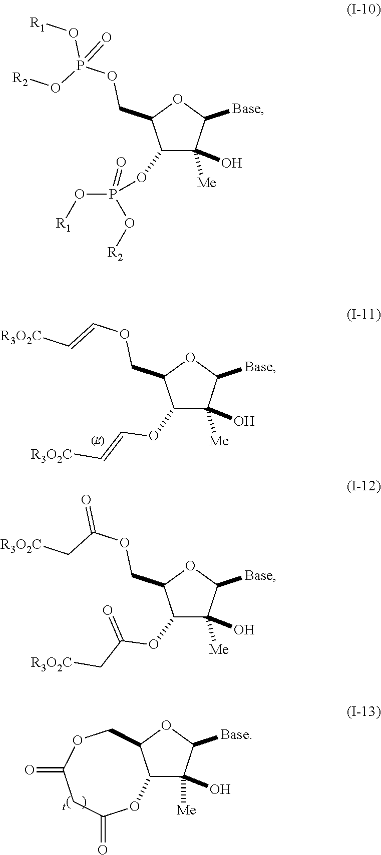

15. The process of any of embodiments 1, 2, 3, 10, 11, 12, 13, wherein the compound of formula (II) is a compound of formula (II-1) or (II-2) or (II-3) or (II-4):

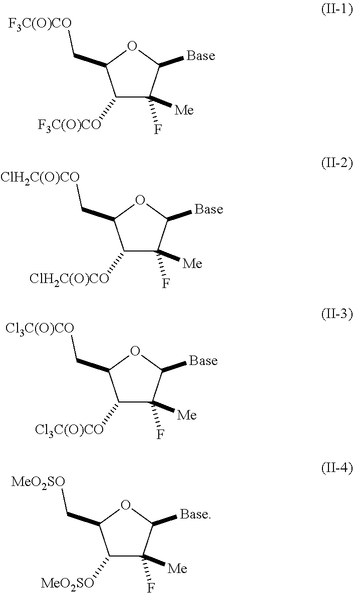

16. The process of any of embodiments 1 to 12, 14, wherein the compound of formula (II) is a compound of formula (II-5) to (II-13)

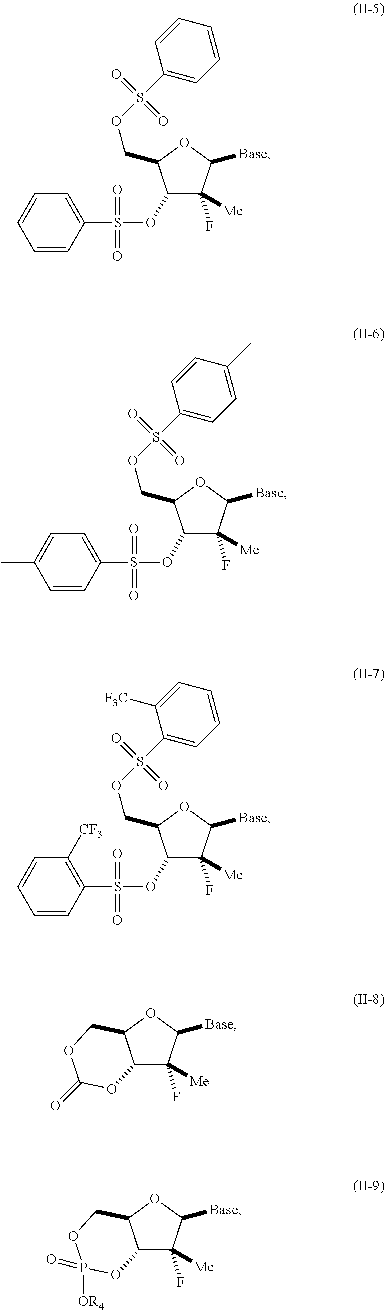

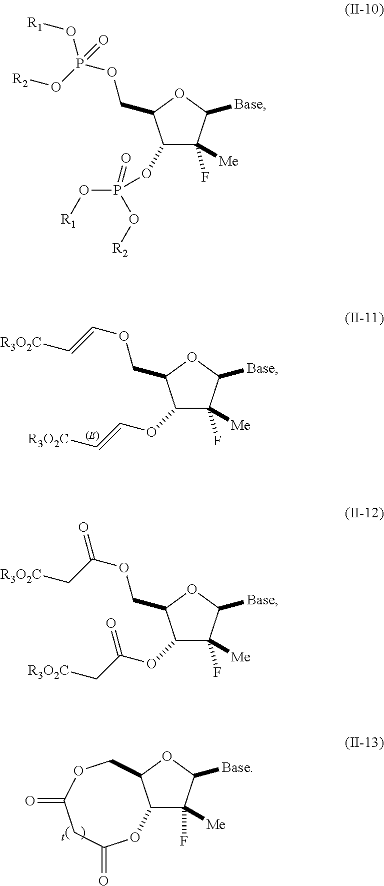

17. The process of any of embodiments 1, 2, 3, 10, 11, 12, 13 or 15, wherein the compound of formula (I) is a compound of formula (I-1′) or (I-2′) or (I-3′) or (I-4′)

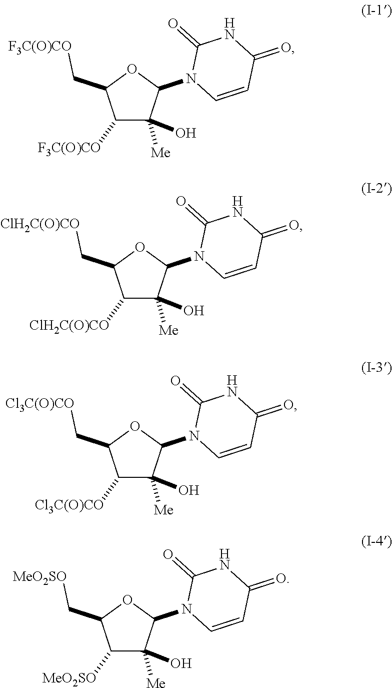

18. The process of any of embodiments 1 to 12 or 14, 16, wherein the compound of formula (I) is a compound of any of formula (I-5′) to (I-13′)

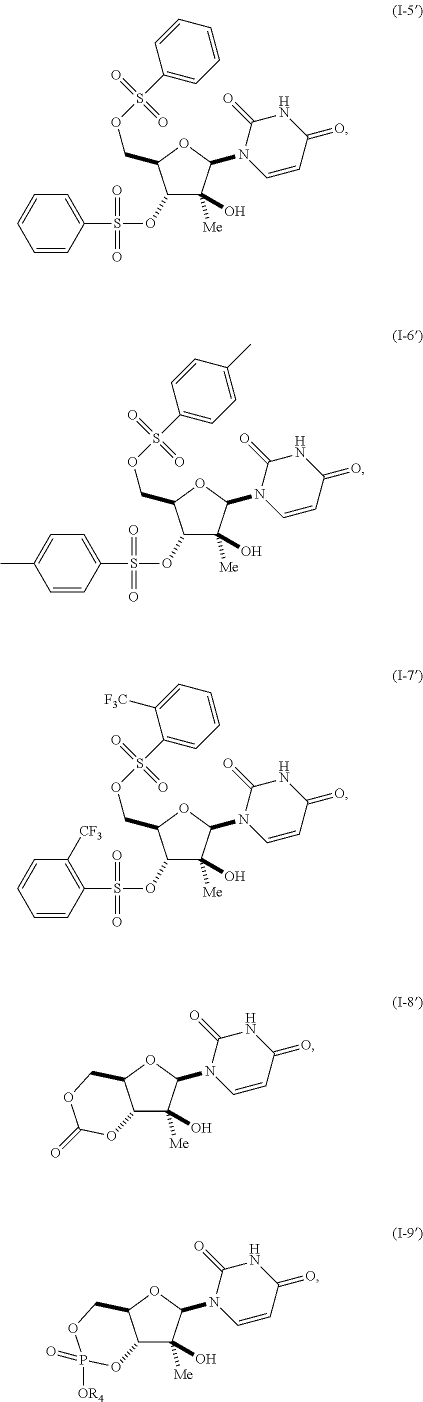

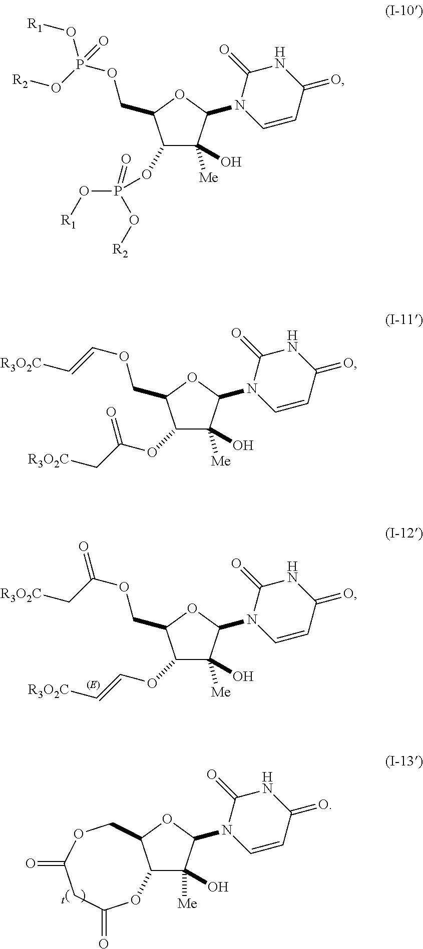

19. The process of any of embodiments 1, 2, 3, 10, 11, 12, 13, 15 or 17, wherein the compound of formula (II) is a compound of formula (II-1′) or (II-2′) or (II-3′) or (II-4′)

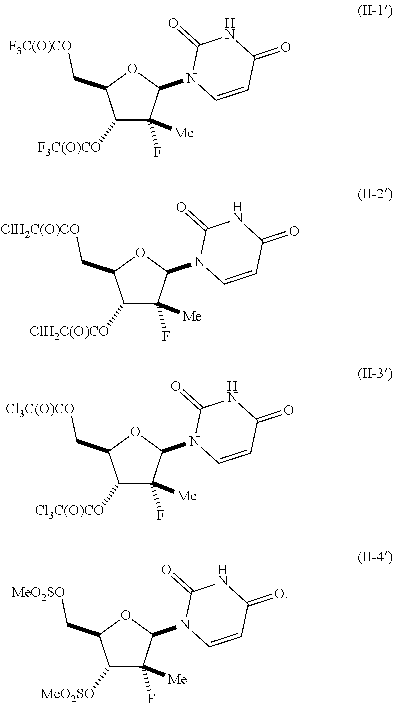

20. The process of any of embodiments 1 to 12, 14, 16 or 18, wherein the compound of formula (II) is a compound of formula (II-5′) to (II-13′)

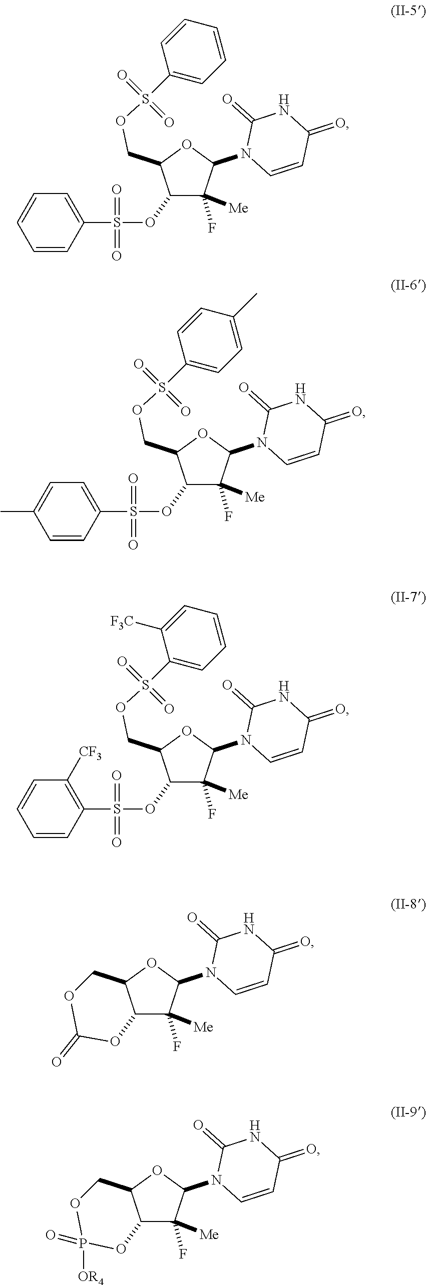

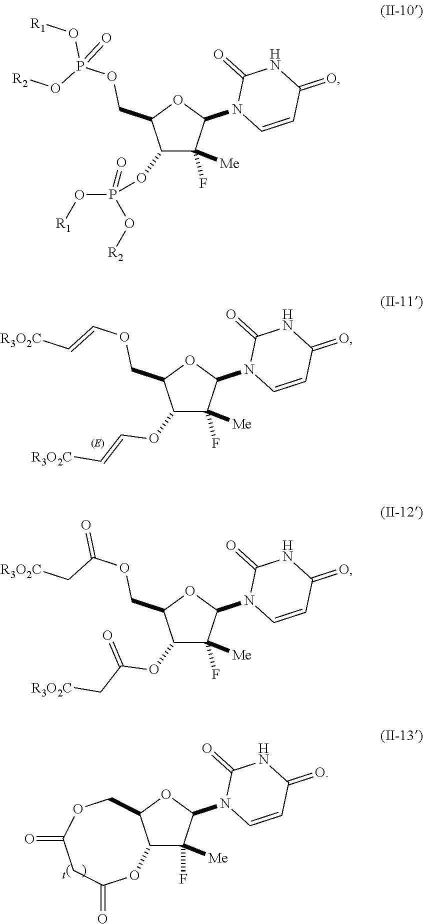

21. The process of any of embodiments 1 to 20, wherein the compound of formula (III) is the compound of formula (III)

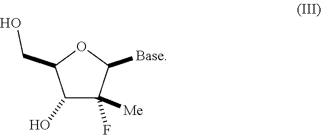

22. The process of any of embodiments 1 to 21, wherein the compound of formula (III) is the compound of formula (III′)

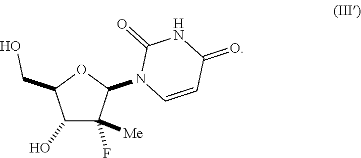 [SMILES: C[C@@]1(F)[C@H](O)[C@@H](CO)O[C@H]1n1ccc(=O)[nH]c1=O]

23. The process of any of embodiments 1 to 22, wherein the mixture provided in (i) further comprises one or more organic solvents.
24. The process of embodiment 23, wherein the one or more organic solvents are one or more aprotic organic solvents, preferably one or more apolar aprotic organic solvents.
25. The process of embodiment 23 or 24, wherein the one or more organic solvents are selected from the group consisting of CH₂Cl₂, dichloroethane, chloroform, toluene, acetone, acetonitrile, 1,4-dioxane, tetrahydrofuran (THF), methyl tetrahydrofuran, methyl tert-butyl ether, methyl ethyl ketone, ethyl acetate, butyl acetate and nitromethane and a mixture of two or more thereof, preferably the one or more organic solvents are selected from the group consisting of CH₂Cl₂, dichloroethane, chloroform, toluene, tetrahydrofuran, methyl tert-butyl ether, 1,4-dioxane and nitromethane and a mixture of two or more thereof.
26. The process of any of embodiments 23 to 25, wherein the solvent is CH₂Cl₂ or tetrahydrofuran, preferably CH₂Cl₂.
27. The process of any of embodiments 23 to 25, wherein the solvent is anhydrous.
28. The process of any of embodiments 1 to 27, wherein the mixture provided in (i) further comprises one or more organic bases, preferably one or more organic tertiary nitrogen bases.
29. The process of embodiment 28, wherein the one or more bases are selected from the group consisting of triethylamine, pyridine, N,N′-diisopropylethylamine, 1,8-diazabicycloundec-7-ene, quinoline, isoquinoline, acridine, pyrazine, and imidazole and a mixture of two or more thereof, preferably one or more of triethylamine, N,N′-diisopropylethylamine, 1,8-diazabicycloundec-7-ene, and pyridine and a mixture of two or more thereof.
30. The process of embodiment 28 or 29, wherein the base is triethylamine.
31. The process of any of embodiments 28 to 30, wherein in the mixture provided in (i), the one or more bases and the compound of formula (I) are present in a molar ratio of the one or more bases relative to the compound of formula (I) in the range of from 0.1:1 to 3:1, preferably in the range of from 0.75:1 to 1.5:1, more preferably in the range of from 0.95:1 to 1.05:1, wherein, if more than one base is comprised in the mixture, the molar ratios relate to the total molar amount of all bases.
32. The process of any of embodiments 1 to 31, wherein the mixture provided in (i) further comprises an agent selected from the group consisting of triethylamine trihydrofluoride (TEA 3HF), triethylamine dihydrofluoride (TEA 2HF), diazabicycloundec-7-ene (DBU), and a mixture of two or more thereof, wherein preferably, the agent is triethylamine trihydrofluoride or triethylamine dihydrofluoride.
33. The process of embodiment 32, wherein the agent and the compound of formula (I) are present in a molar ratio of the agent relative to the compound of formula (I) in the range of from 0.1:1 to 3:1, preferably in the range of from 1.75:1 to 2.5:1, more preferably in the range of from 1.95:1 to 2.05:1.
34. The process of any of embodiments 1 to 33, wherein the mixture provided in (i) is provided in an inert gas atmosphere, preferably an inert atmosphere comprising nitrogen.
35. The process of any of embodiments 23 to 34, wherein the mixture comprising a compound of formula (I) provided in (i) further comprises the one or more solvents, the agent and optionally the one or more bases.
36. The process of any of embodiments 23 to 35, wherein the mixture comprising a compound of formula (I) provided in (i) further comprises diethylamino(difluoro)sulfonium tetrafluoroborate or difluoro(morpholino)sulfonium tetrafluoroborate, the one or more solvents, the agent and optionally the one or more bases.
37. The process of any of embodiments 1 to 36, wherein prior to (ii) the diethylamino(difluoro)sulfoniumtetrafluoroborate or the difluoro(morpholino)sulfonium tetrafluoroborate is present in a molar ratio of the diethylamino(difluoro)sulfoniumtetrafluoroborate or the difluoro(morpholino)sulfonium tetrafluoroborate relative to the compound of formula (I) in the range of from 0.1:1 to 3:1, preferably in the range of from 1.25:1 to 2.0:1, more preferably in the range of from 1.45:1 to 1.65:1.
38. The process of any of embodiments 1 to 37, wherein the temperature during (ii) is in the range of from −80 to 40° C., preferably in the range of from 20 to 30° C., more preferably in the range of from 20 to 25° C.
39. The process of any of embodiments 1 to 38, wherein according to (ii) the mixture is subjected to the fluorination conditions for a period of time in the range of from 0.5 to 24 h, preferably in the range of from 0.5 to 2 h, more preferably in the range of from 0.5 to 1.5 h.
40. The process of any of embodiments 1 to 39, wherein prior to (iii), the process further comprises
   (ii′) separating the compound of formula (II) from the mixture obtained in (ii).
41. The process of embodiment 40, wherein the separating in (ii′) comprises
   (ii′-1) extracting the compound of formula (II) from the mixture obtained in (ii); and
   (ii′-2) separating the compound of formula (II) from the mixture obtained in (ii′-1).
42. The process of embodiment 40 or 41, wherein the separating according to (ii′) or the separating according to (ii′-2) comprises filtration, centrifugation, drying, or a combination of two or more thereof.
43. The process according to any of embodiments 1 to 42, wherein (iii) further comprises adding to the mixture obtained in (ii) one or more deprotection reagents selected from the group consisting of water, a mixture of NH₃ and MeOH, and a mixture of NaOMe and MeOH.
44. The process of any of embodiments 1 to 43, wherein the deprotection conditions in (iii) comprise a temperature of the mixture during deprotection in (iii) in the range of from 15 to 35° C., preferably in the range of from 20 to 30° C., more preferably in the range of from 20 to 25° C.
45. The process of any of embodiments 1 to 44, wherein according to (iii) the mixture is subjected to the deprotection conditions for a period of time in the range of from less than 1 to 120 min, preferably in the range of from less than 1 to 50 min.
46. The process of any of embodiments 1 to 45, which further comprises
   (iv) separating the compound of formula (III) from the mixture obtained in step (iii).
47. The process of embodiment 46, wherein the separating in (iv) comprises
   (iv-1) extracting the compound of formula (III) from the mixture obtained in (iii), and
   (iv-2) separating the compound of formula (III) from the mixture obtained in (iv-1).
48. The process of embodiment 46 or 47, wherein the separating according to (iv) or the separating according to (iv-2) comprises filtration, centrifugation, drying, or a combination of two or more thereof.
49. The process of any of embodiments 46 to 48, wherein the separating according to (iv) or according to (iv-2) comprises
   (iv-1′) crystallizing the compound of formula (III) from the mixture obtained in (iv) or in (iv-2),
   (iv-2′) separating the compound of formula (III) in the mixture obtained from (iv-1′) from its mother liquor.
50. The process of embodiment 49, wherein the crystallizing according to (iv-1′) comprises seeding with seeds of the compound of formula (III).
51. The process of embodiment 49 or 50, wherein crystallizing according to (iv-1′) is carried out in a solvent selected from the group consisting of ethyl acetate, isopropanol or tetrahydrofuran.
52. The process of any of embodiments 1 to 51, further comprising providing the mixture according to (i) by a process comprising
   (a) providing a mixture comprising a compound of formula (IV)

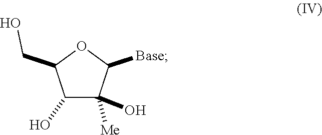

(b) subjecting the mixture provided in (a) to protection conditions in the presence of an OH-protecting agent comprising an inert electron-withdrawing OH-protecting group R, obtaining a mixture comprising the compound of formula (I)

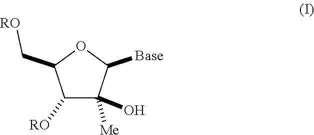

wherein at each occurrence
R is an inert electron withdrawing OH protecting group; and
Base is a purinyl residue or a pyrimidinyl residue linked to the furanose ring according to formulae (I) and (IV) through a carbon or a nitrogen atom.
53. A process for preparing a mixture comprising a compound of formula (I) comprising
   (a) providing a mixture comprising a compound of formula (IV)

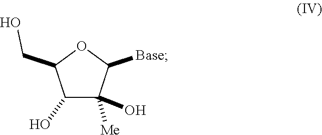

(b) subjecting the mixture provided in (a) to protection conditions in the presence of an OH-protecting agent comprising an inert electron-withdrawing OH-protecting group R, obtaining a mixture comprising a compound of formula (I)

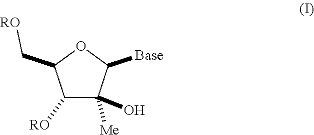

wherein at each occurrence
R is an inert electron withdrawing OH protecting group; and
Base is a purinyl residue or a pyrimidinyl residue linked to the furanose ring according
to formulae (I) and (IV) through a carbon or a nitrogen atom.
54. A process for preparing a compound of formula (I) comprising
   (a) providing a mixture comprising a compound of formula (IV)

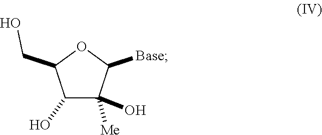

(b) subjecting the mixture provided in (a) to protection conditions in the presence of an OH-protecting agent comprising an inert electron-withdrawing OH-protecting group R, obtaining a compound of formula (I)

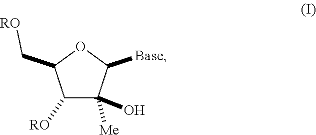

wherein at each occurrence
   R is an inert electron withdrawing OH protecting group; and
   Base is a purinyl residue or a pyrimidinyl residue linked to the furanose ring according to formulae (I) and (IV) through a carbon or nitrogen atom.
55. The process of any of embodiments 52 to 54, wherein R is selected from the group consisting of X₃₋ₙHₙCC(O) wherein X is halogen and n is 0, 1, or 2; or R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).
56. The process of any of embodiments 52 to 54, wherein
   R is selected from SO₂Ph or SO₂-o-CF₃-Ph (ortho-trifluoromethylphenyl); or
   R is

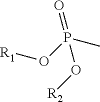

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or
R is selected from CH═CH₂—CO₂R₃ or C(O)—CH₂—CO₂R₃ wherein R₃ is selected from the group consisting of alkyl, aryl and cycloalkyl; or
wherein the radical R attached to the oxygen in position 5′ of the sugar moiety taken together with the radical R attached to the oxygen in position 3′ of the sugar moiety forms
a group selected from C(O), C(O)—(CH₂)ₜ—CO or

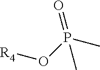

wherein R₄ is selected from the group consisting of alkyl, aryl and cycloalkyl and wherein t is 1 or 2.
57. The process of embodiment 56, wherein R₁ is C₁-C₆ alkyl, preferably C₁-C₄ alkyl, more preferably C₁-C₂ alkyl, or R₁ is an aryl selected from phenyl or naphtyl.
58. The process of embodiment 56 or 57, wherein R₂ is C₁-C₆ alkyl, preferably C₁-C₄ alkyl, more preferably C₁-C₂ alkyl, or R₂ is a C₃-C₆ cycloalkyl, preferably is a C₅-C₆ cycloalkyl or R₂ is an aryl selected from phenyl or naphtyl.
59. The process of embodiment 56, wherein R₃ is C₁-C₆ alkyl, preferably C₁-C₄ alkyl, more preferably C₁-C₂ alkyl, or R₃ is a C₃-C₆ cycloalkyl, preferably is a C₅-C₆ cycloalkyl or R₃ is an aryl selected from phenyl or naphtyl.
60. The process of any of embodiment 56, wherein R₄ is C₁-C₆ alkyl, preferably C₁-C₄ alkyl, more preferably C₁-C₂ alkyl, or R₄ is a C₃-C₆ cycloalkyl, preferably is a C₅-C₆ cycloalkyl or R₄ is an aryl selected from phenyl or naphtyl.
61. The process of any of embodiments 56 to 58, wherein q is selected from 2, 3 and 4.
62. The process of any of embodiments 52 to 55, wherein R is selected from the group consisting of F₃CC(O), Cl₃CC(O), ClH₂CC(O), Cl₂HCC(O), F₂HCC(O), FH₂CC(O) and SO₂Me, preferably C(O)CCl₃, C(O)CF₃, C(O)CH₂Cl, more preferably C(O)CCl₃, C(O)CH₂Cl, and the agent is Cl—C(O)CCl₃, Cl—C(O)CF₃, O(C(O)CF₃)₂Cl—C(O)CH₂Cl, O(C(O)CH₂Cl)₂Cl₂HCC(O)—Cl, F₂HCC(O)—Cl, FH₂CC(O)—Cl or Cl—SO₂Me, preferably the agent is Cl—C(O)CCl₃, O(C(O)CF₃)₂ or O(C(O)CH₂Cl)₂.
63. The process of any of embodiments 52 to 62, wherein Base is selected from the group consisting of uridine, protected uridine, thymine, protected thymine, cytidine, protected cytidine, adenosine, protected adenosine, guanine and protected guanine.
64. The process of any of embodiments 52 to 63, wherein Base is uridine.
65. The process of any of embodiments 52 to 64, wherein the mixture provided in (a) comprises one or more organic solvents.
66. The process of embodiment 65, wherein the one or more organic solvents are one or more polar organic solvents.
67. The process of any of embodiments 65 or 66, wherein the one or more organic solvents are selected from the group consisting of CH₂Cl₂, pyridine toluene, acetone, acetonitrile, dioxane, tetrahydrofuran (THF), methyl tetrahydrofuran, methyl ethyl ketone, ethyl acetate, butyl acetate and dimethylformamide and a mixture of two or more thereof.
68. The process of any of embodiments 65 to 67, wherein the solvent is CH₂Cl₂, tetrahydrofuran, pyridine or dimethylformamide, preferably pyridine or dimethylformamide and a mixture of two or more thereof.
69. The process of any of embodiments 65 to 68, wherein the solvent is anhydrous.
70. The process of any of embodiments 52 to 69, wherein the mixture provided in (a) further comprises one or more organic or one or more inorganic bases or mixtures of two or more thereof.
71. The process of embodiment 70, wherein the mixture provided in (a) comprises one or more inorganic bases, preferably comprising a carbonate, more preferably an alkali metal carbonate, more preferably sodium carbonate.
72. The process of embodiment 70, wherein the mixture provided in (a) comprises one or more organic bases, preferably one or more organic tertiary nitrogen bases.
73. The process of embodiment 70 or 72, wherein the one or more bases are one or more of pyridine, 2,6 dimethylpyridine, triethylamine N,N′-diisopropylethylamine, 1,8-diazabicycloundec-7-ene, quinoline, isoquinoline, acridine, pyrazine, and imidazole, preferably one or more of triethylamine, N,N′-diisopropylethylamine, 1,8-diazabicycloundec-7-ene, and pyridine and mixtures of two or more thereof.
74. The process of embodiment 73, wherein the base is pyridine or 2,6 dimethylpyridine.
75. The process of any of embodiments 70 to 74, wherein in the mixture provided in (a), the one or more bases and the compound of formula (IV) are present in a molar ratio of the one or more bases relative to the compound of formula (IV) in the range of from 3:1 to 30:1, preferably in the range of from 10:1 to 25:1, more preferably in the range of from 17:1 to 22:1, wherein, if more than one base is comprised in the mixture, the molar ratios relate to the total molar amount of all bases.
76. The process of any of embodiments 52 to 75, wherein the mixture provided in (a) further comprises a reagent selected from the group consisting of N,N-dialkylaminopyridines and pyridine, preferably wherein the N,N-dialkylaminopyridine is N,N-dimethylaminopyridine (DMAP).
77. The process of embodiment 76, wherein the reagent selected from the group consisting of N,N-dialkylaminopyridines and pyridine, preferably wherein the N,N-dialkylaminopyridine is N,N-dimethylaminopyridine (DMAP), and the compound of formula (IV) are present in a molar ratio of reagent selected from the group consisting of N,N-dialkylaminopyridines and pyridine, preferably wherein the N,N-dialkylaminopyridine is N,N-dimethylaminopyridine (DMAP), relative to the compound of formula (IV), in the range of from 0.1:1 to 0.6:1.
78. The process of any of embodiments 52 to 77, wherein the temperature during (b) is in the range of from 15 to 35° C., preferably in the range of from 20 to 30° C.
79. The process of any of embodiments 52 to 78, wherein according to (b) the mixture is subjected to the protection conditions for a period of time in the range of from 1 to 24 h, preferably in the range of from 2 to 20 h.
80. The process of any of embodiments 52 to 79, wherein prior to (b), the protecting agent and the compound of formula (IV) are present in a molar ratio of protecting agent relative to the compound of formula (IV) in the range of from 1:1 to 10:1, preferably in the range of from 2:1 to 9:1, more preferably in the range of from 2.5:1 to 7:1.
81. The process of any of embodiments 52 to 80, which further comprises
   (c) separating the compound of formula (I) from the mixture obtained in (b).
82. The process of embodiment 81, wherein the separating in (c) comprises
   (c-1) extracting the compound of formula (I) from the mixture obtained in (b), and
   (c-2) separating the compound of formula (I) from the mixture obtained in (c-1).
83. The process of embodiment 81 or 82, wherein the separating according to (c) or the separating according to (c-2) comprises filtration, centrifugation, drying, or a mixture of two or more thereof.
84. The process of any of embodiments 52 to 83, wherein the mixture provided in (a) is provided in an inert gas atmosphere, preferably an inert atmosphere comprising nitrogen.
85. A compound of formula (I) or a compound of formula II)

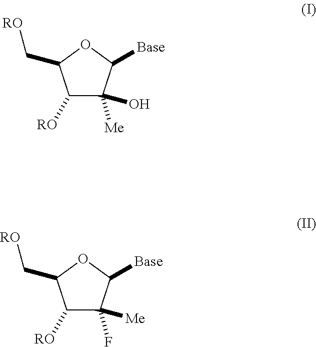

wherein at each occurrence
   R is an inert electron withdrawing OH protecting group; and
   Base is a purinyl residue or a pyrimidinyl residue linked to the furanose ring according to formulae (I) and (II)) through a carbon or a nitrogen atom.
86. The compound of formula (I) or the compound of formula (II) of embodiment 85, wherein
   R is selected from the group consisting of X₃₋ₙHₙCC(O)
      wherein X is halogen and n is 0, 1, or 2; or
   R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).
87. The compound of formula (I) or the compound of formula (II) of embodiment 85, wherein
   R is selected from SO₂Ph or SO₂-o-CF₃-Ph (ortho-trifluoromethylphenyl); or
   R is

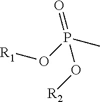

wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or
R is selected from CH═CH₂—CO₂R₃ or C(O)—CH₂—CO₂R₃ wherein R₃ is selected from the group consisting of alkyl, aryl and cycloalkyl; or
wherein the radical R attached to the oxygen in position 5′ of the sugar moiety taken together with the radical R attached to the oxygen in position 3′ of the sugar moiety forms
a group selected from C(O), C(O)—(CH₂)ₜ—CO or

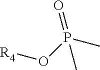

wherein R₄ is selected from the group consisting of alkyl, aryl and cycloalkyl and wherein t is 1 or 2.
88. The compound of embodiment 87, wherein R₁ is C₁-C₆ alkyl, preferably C₁-C₄ alkyl, more preferably C₁-C₂ alkyl, or R₁ is an aryl selected from phenyl or naphtyl.
89. The compound of embodiment 87 or 88, wherein R₂ is C₁-C₆ alkyl, preferably C₁-C₄ alkyl, more preferably C₁-C₂ alkyl, or R₂ is a C₃-C₆ cycloalkyl, preferably is a C₅-C₆ cycloalkyl or R₂ is an aryl selected from phenyl or naphtyl.
90. The compound of embodiments 87, wherein R₃ is C₁-C₆ alkyl, preferably C₁-C₄ alkyl, more preferably C₁-C₂ alkyl, or R₃ is a C₃-C₆ cycloalkyl, preferably is a C₅-C₆ cycloalkyl or R₃ is an aryl selected from phenyl or naphtyl.
91. The compound of embodiment 87, wherein R₄ is C₁-C₆ alkyl, preferably C₁-C₄ alkyl, more preferably C₁-C₂ alkyl, or R₄ is a C₃-C₆ cycloalkyl, preferably is a C₅-C₆ cycloalkyl or R₄ is an aryl selected from phenyl or naphtyl.
92. The compound of any of embodiments 87 to 89, wherein q is selected from 2, 3 and 4.
93. The compound of formula (I) or the compound of formula (II) of embodiment 85 or 86, wherein R is selected from the group consisting of F₃CC(O), Cl₃CC(O), ClH₂CC(O), Cl₂HCC(O), F₂HCC(O), FH₂CC(O) and SO₂Me.
94. The compound of formula (I) or the compound of formula (II) of any of embodiments 85 to 93, wherein Base is selected from the group consisting of uridine, protected uridine, thymine, protected thymine, cytidine, protected cytidine, adenosine, protected adenosine, guanine and protected guanine.
95. The compound of formula (I) or the compound of formula (II) of any of embodiments 85 to 94, wherein Base is uridine.
96. The compound of formula (I) of any of embodiments 85, 86, 93 or 94, wherein the compound of formula (I) is the compound of formula (I-1) or (I-2) or (I-3) or (I-4):

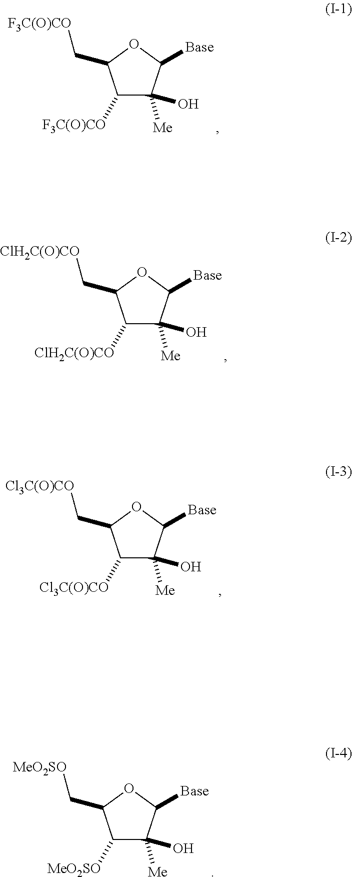

97. The compound of formula (I) of any of embodiments 85 to 92, 94, 95, wherein the compound of formula (I) is the compound of any of formula (I-5) to (I-13)

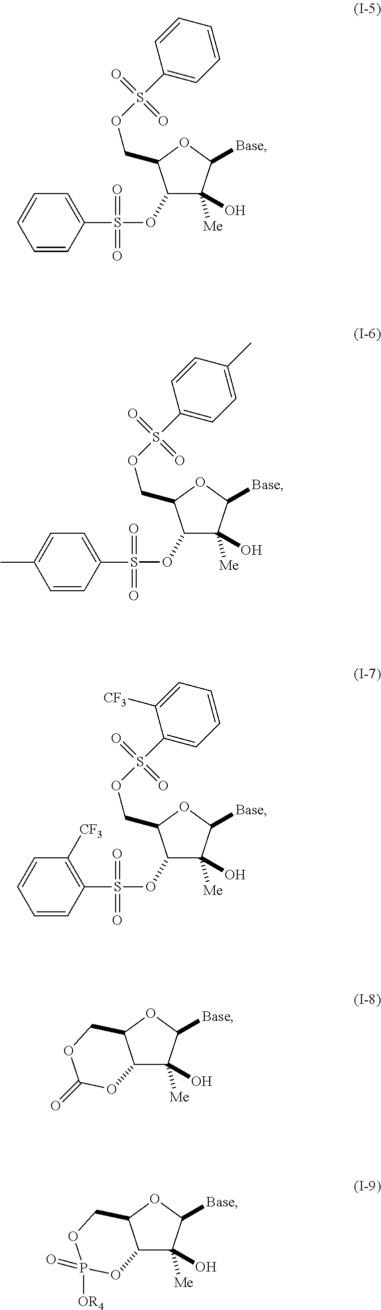

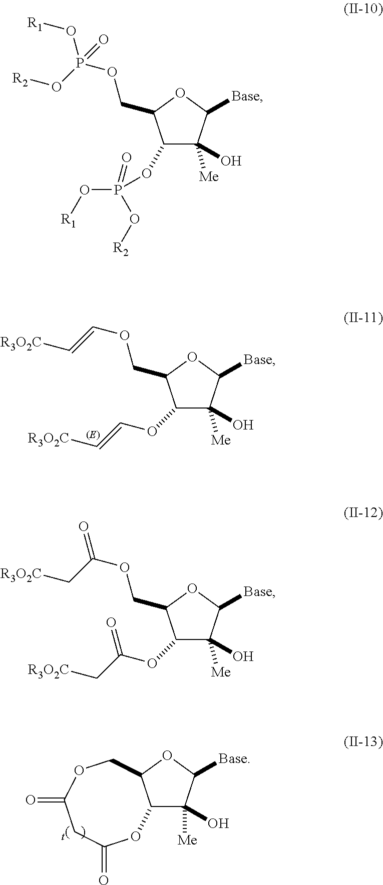

98. The compound of formula (II) of any of embodiments 85, 86, 93, 94, 95, wherein the compound of formula (II) is the compound of formula (II-1) or (II-2) or (II-3) or (II-4):

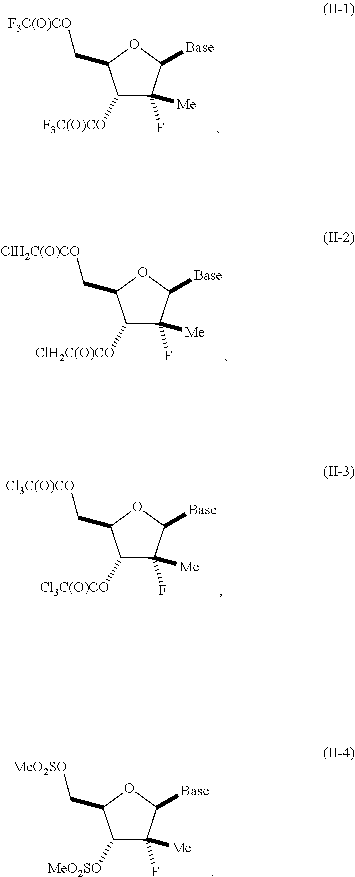

99. The compound of formula (II) of any of embodiments 85 to 92, 94, 95, wherein the compound of formula (II) is the compound of any of formulae (II-5) to (II-13)

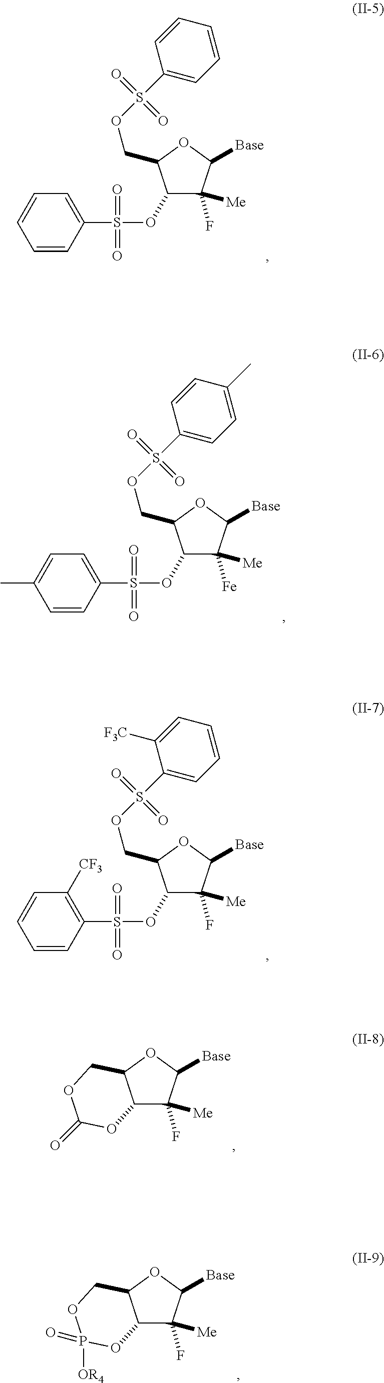

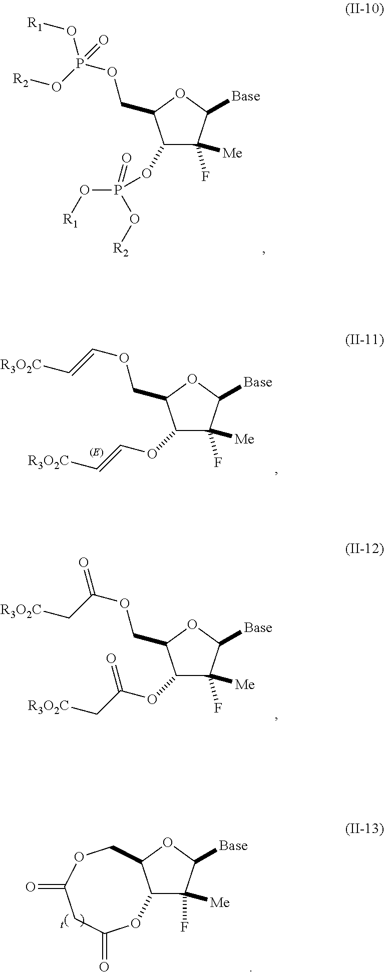

100. The compound of formula (I) of any of embodiments 85, 86, 93, 94, 95, 96, 98, wherein the compound of formula (I) is the compound of any of formula (I-1′) or (I-2′) or (I-3′) or (I-4′):

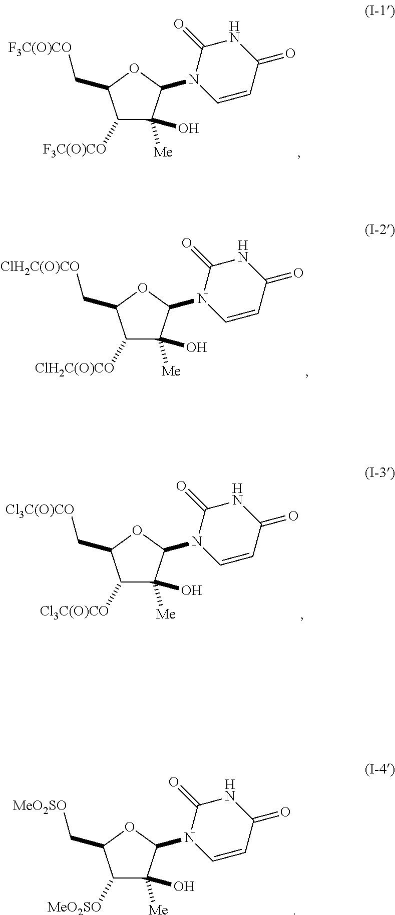

101. The compound of formula (I) of any of embodiments 85 to 92, 94, 95, 97, 99, wherein the compound of formula (I) is the compound of any of formulae (I-5′) to (I-13′)

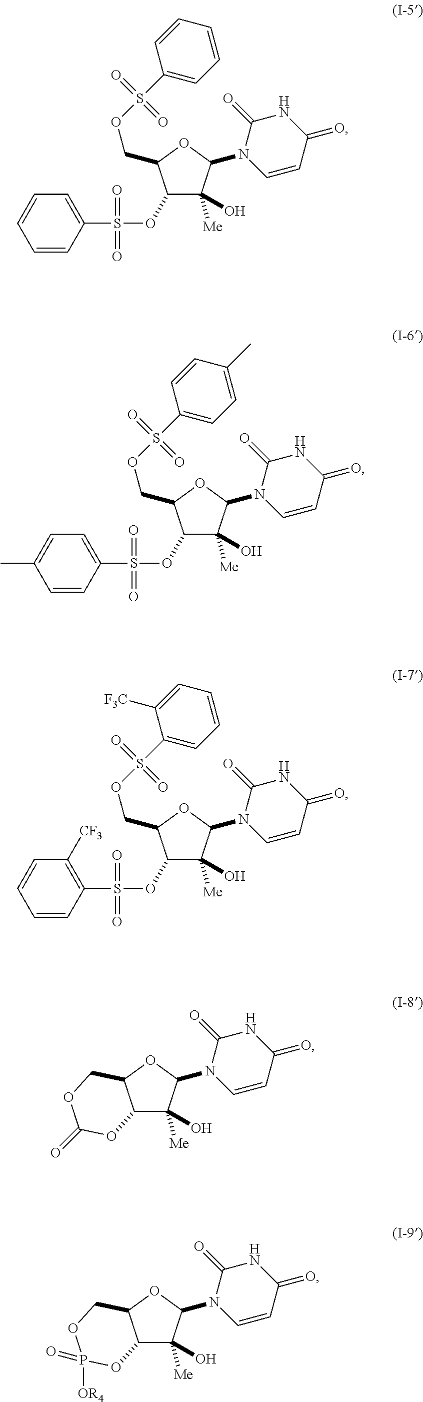

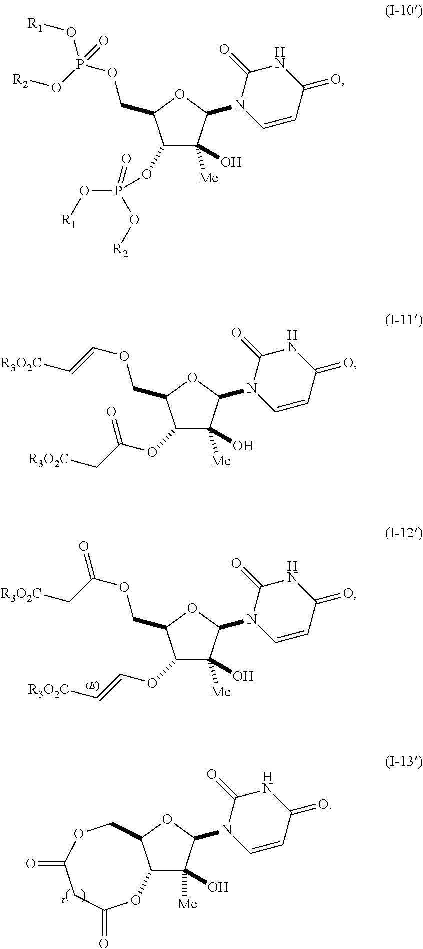

102. The compound of formula (II) of any of embodiments 85, 86, 93 to 96, 98, 100, wherein the compound of formula (II) is the compound of any of formula (II-1′) or (II-2′) or (II-3′) or (II-4′):

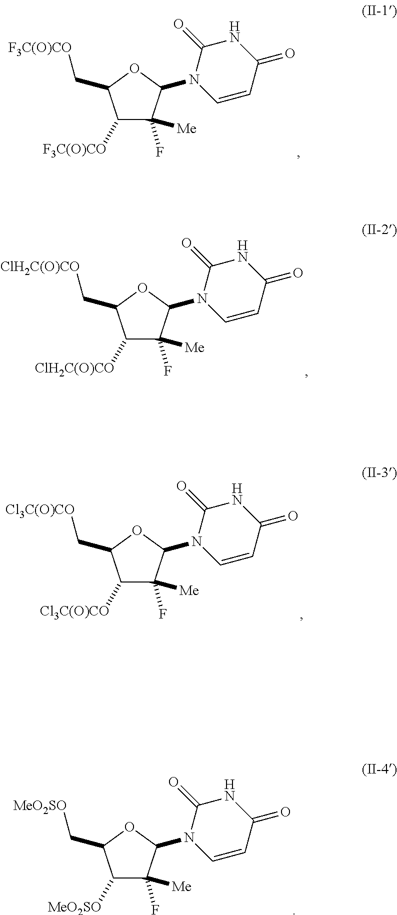

103. The compound of formula (II) of any of embodiments 85 to 92, 94 to 95, 97, 99, 101, wherein the compound of formula (II) is the compound of any of formula (II-5′) to (I-13′)

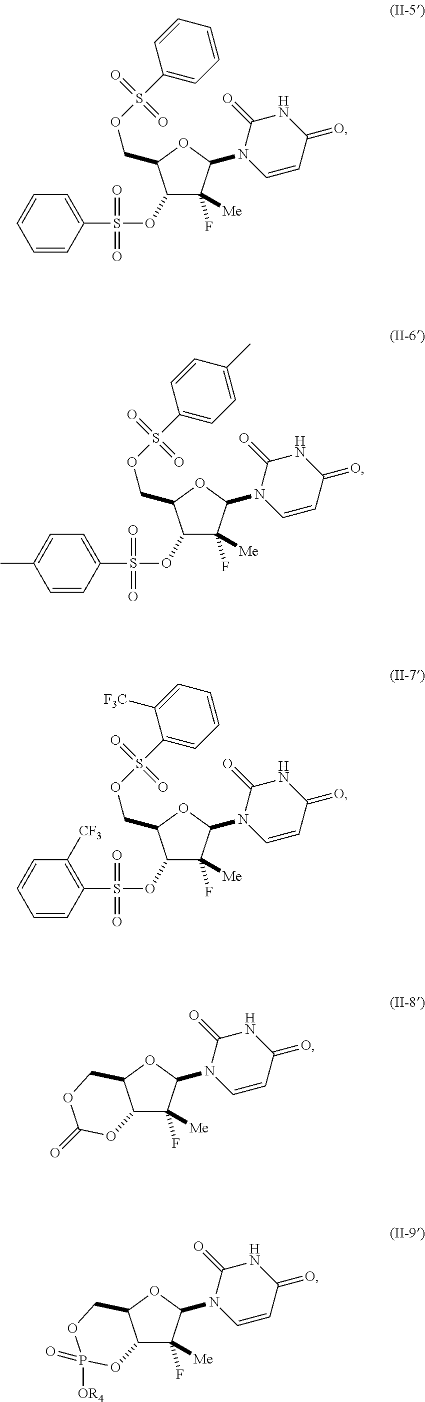

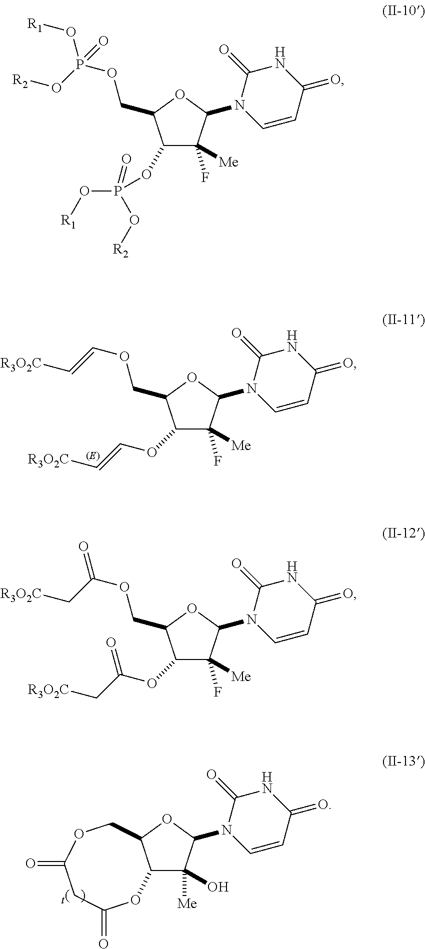

104. A mixture comprising a compound of formula (I) of any of embodiments 85 to 97, 100, 101, obtained or obtainable by a process according to any of embodiments 52 to 84.
105. A mixture comprising a compound of formula (II) of any of embodiments 85 to 95, 98, 99, 102, 103 obtained or obtainable by a process according to any of embodiments 1 to 84.
106. Use of a compound of formula (I) according to any of embodiments 85 to 97, 100, 101, as a reagent in the preparation of a 2′ fluoro-nucleoside phosphoramidate.
107. The use of embodiment 106, wherein the 2′ fluoro-nucleoside phosphoramidate is sofosbuvir of the formula (X′)

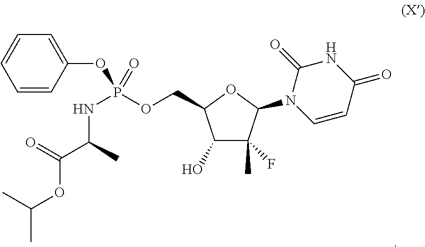 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

108. Use of a compound of formula (II) according to any of embodiments 85 to 95, 98, 99, 102 or 103, as a reagent in the preparation of a 2′ fluoro-nucleoside phosphoramidate.
109. The use of embodiment 108, wherein the 2′ fluoro-nucleoside phosphoramidate is sofosbuvir of the formula (X′)

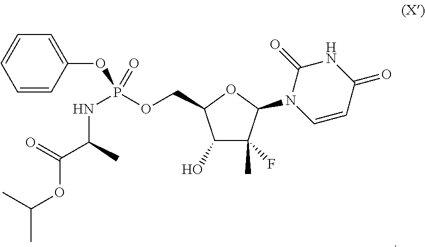 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

110. The use of embodiment 108 or 109, wherein the compound of formula (II) is comprised in a mixture obtainable or obtained by a process according to any of embodiments 1 to 84.
111. A process for the preparation of sofosbuvir of the formula (X′)

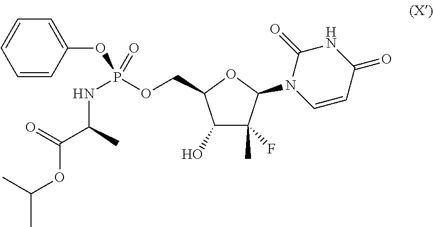 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

comprising
(x) reacting a compound of formula (II) according to any of embodiments 85 to 95, 98, 99, 102, 103 and obtaining a compound of formula (III); and
(xx) reacting the compound of formula (III) and obtaining sofosbuvir of formula (X′).
112. A process for the preparation of sofosbuvir of the formula

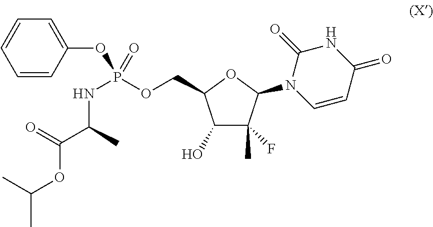 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

comprising
(xx′) reacting the compound of formula (III′)

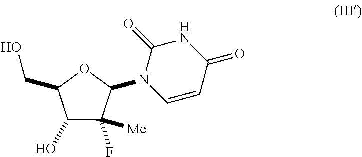 [SMILES: C[C@@]1(F)[C@H](O)[C@@H](CO)O[C@H]1n1ccc(=O)[nH]c1=O]

to sofosbuvir of formula (X′).
113. A mixture comprising a compound of formula (II) according to any of embodiments 85 to 95, 98, 99, 102, 103.
114. A mixture comprising a compound of formula (II) having a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

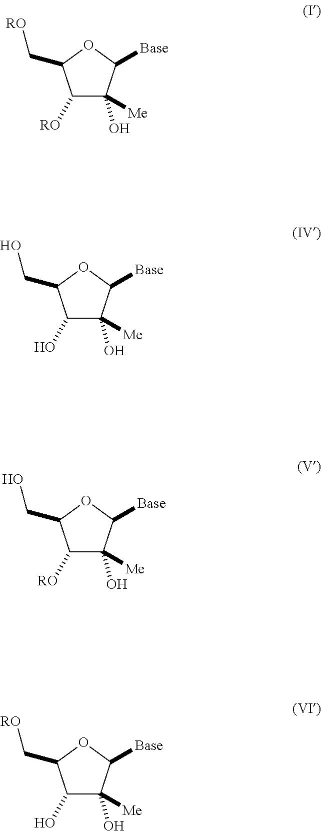

wherein at each occurrence
R is an inert electron withdrawing OH protecting group; and
Base is a purinyl residue or a pyrimidinyl residue linked to the furanose ring according to formulae (II), (I′), (IV′), (V′) and (VI′) through a carbon or a nitrogen atom; wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).
115. A mixture comprising a compound of formula (II) obtainable or obtained by a process according to any of embodiments 1 to 84 having a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

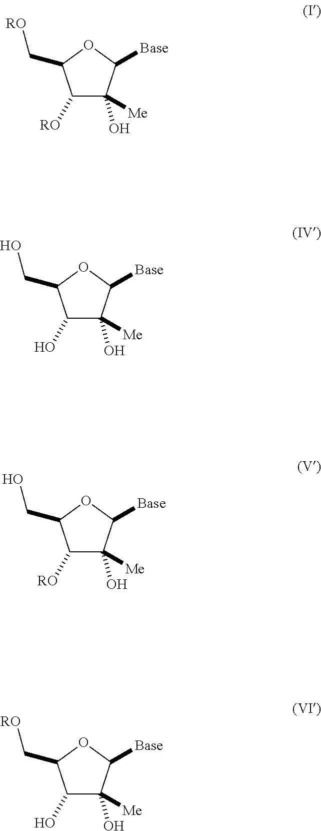

wherein at each occurrence
R is an inert electron withdrawing OH protecting group; and
Base is a purinyl residue or a pyrimidinyl residue linked to the furanose ring according to formulae (II), (I′), (IV′), (V′) and (VI′) through a carbon or a nitrogen atom, wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).
116. A mixture comprising a compound of formula (III) obtainable or obtained by a process according to any of embodiments 1 to 84 having a content based on the weight of the mixture, of at most 1000 weight-ppm, preferably less than 100 weight-ppm, more preferably free of one or more compounds of formula (I′) or one or more compounds of formula (IV′) or one or more compounds of formula (V′) or one or more compounds of formula (VI′) or mixtures of two or more thereof

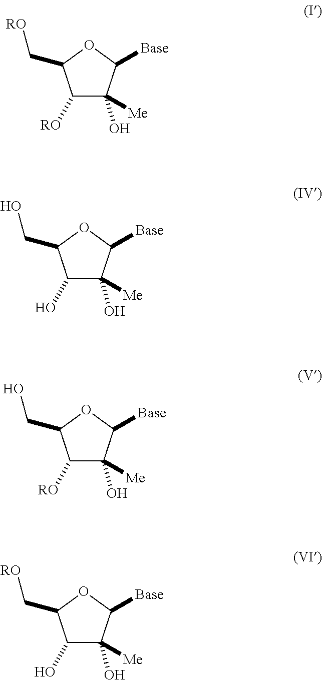

wherein at each occurrence
R is an inert electron withdrawing OH protecting group; and
Base is a purinyl residue or a pyrimidinyl residue linked to the furanose ring according to formulae (II), (I′), (IV′), (V′) and (VI′) through a carbon or nitrogen atom,
wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).
117. The mixture of any of embodiments 113 to 116, wherein
   R is selected from the group consisting of X₃₋ₙHₙCC(O)
      wherein X is halogen and n is 0, 1, or 2; or
   R is selected from the group consisting of SO₂Me, SO₂-p-Me-Ph (tosyl), SO₂-p-NO₂-Ph (paranosyl), SO₂-o-NO₂-Ph (ortho-nosyl) and SO₂CF₃ (triflyl).
118. The mixture of any of embodiments 113 to 116, wherein
   R is selected from SO₂Ph or SO₂-o-CF₃-Ph (ortho-trifluoromethylphenyl); or
   R is

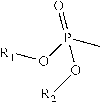

wherein R₁ and R₂ are independently selected from alkyl, aryl or R₁ and R₂ taken together are a —(CH₂—)_{q} group that forms a ring with the oxygen atoms to which R₁ and R₂ are bound and wherein q is 2, 3, 4, 5, 6, 7; or
R is selected from —CH═CH₂—CO₂R₃ or —C(O)—CH₂—CO₂R₃ wherein R₃ is selected from the group consisting of alkyl, aryl and cycloalkyl; or
wherein the radical R attached to the oxygen in position 5′ of the sugar moiety taken together with the radical R attached to the oxygen in position 3′ of the sugar moiety forms
a group selected from C(O), C(O)—(CH₂)ₜ—CO or

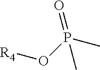

wherein R₄ is selected from the group consisting of alkyl, aryl and cycloalkyl and wherein t is 1 or 2.
119. The mixture of embodiment 118, wherein R₁ is C₁-C₆ alkyl, preferably C₁-C₄ alkyl, more preferably C₁-C₂ alkyl, or R₁ is an aryl selected from phenyl or naphtyl.
120. The mixture of embodiment 118 or 119, wherein R₂ is C₁-C₆ alkyl, preferably C₁-C₄ alkyl, more preferably C₁-C₂ alkyl, or R₂ is a C₃-C₆ cycloalkyl, preferably is a C₅-C₆ cycloalkyl or R₂ is an aryl selected from phenyl or naphtyl.
121. The mixture of any of embodiments 118, wherein R₃ is C₁-C₆ alkyl, preferably C₁-C₄ alkyl, more preferably C₁-C₂ alkyl, or R₃ is a C₃-C₆ cycloalkyl, preferably is a C₅-C₆ cycloalkyl or R₃ is an aryl selected from phenyl or naphtyl.
122. The mixture of any of embodiments 118, wherein R₄ is C₁-C₆ alkyl, preferably C₁-C₄ alkyl, more preferably C₁-C₂ alkyl, or R₄ is a C₃-C₆ cycloalkyl, preferably is a C₅-C₆ cycloalkyl or R₄ is an aryl selected from phenyl or naphtyl.
123. The mixture of any of embodiments 118 to 120 wherein q is selected from 2, 3 and 4.
124. The mixture of any of embodiments 113 to 117, wherein R is selected from the group consisting of F₃CC(O), Cl₃CC(O), ClH₂CC(O), Cl₂HCC(O), F₂HCC(O), FH₂CC(O) and SO₂Me.
125. The mixture of any of embodiments 113 to 124, wherein Base is selected from the group consisting of uridine, protected uridine, thymine, protected thymine, cytidine, protected cytidine adenosine, protected adenosine, guanine and protected guanine.
126. The mixture of any of embodiments 113 to 125, wherein Base is uridine.
127. The process of any of embodiments 1 to 3, 10 to 13, 15, 17, 19, 21 to 55, 62 to 84, wherein the compound of formula (I) is a compound of formula (I-1)

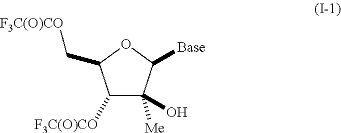

128. The process of any of embodiments 1 to 3, 10 to 13, 15, 17, 19, 21 to 55, 62 to 84, wherein the compound of formula (I) is a compound of formula (I-2)

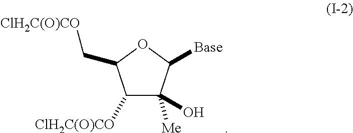

129. The process of any of embodiments 1 to 3, 10 to 13, 15, 17, 19, 21 to 55, 62 to 84, wherein the compound of formula (I) is a compound of formula (I-2)

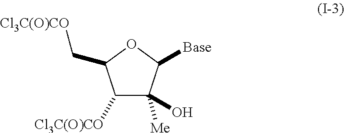

130. The process of any of embodiments 1 to 3, 10 to 13, 15, 17, 19, 21 to 55, 62 to 84, wherein the compound of formula (I) is a compound of formula (I-4)

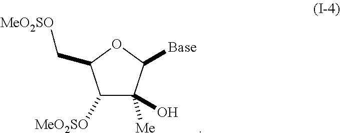

131. The process of any of embodiments 1, 2, 4, 11, 12, 14, 16, 18, 20, 21 to 54, 56, 60, 63 to 84, wherein the compound of formula (I) is a compound of formula (I-5)

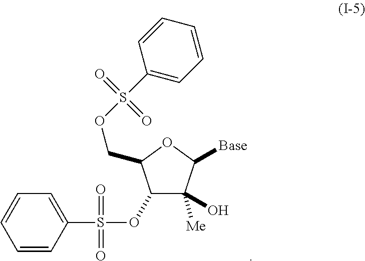

132. The process of any of embodiments 1 to 3, 11, 12, 14, 16, 18, 20, 21 to 55, 63 to 84, wherein the compound of formula (I) is a compound of formula (I-6)

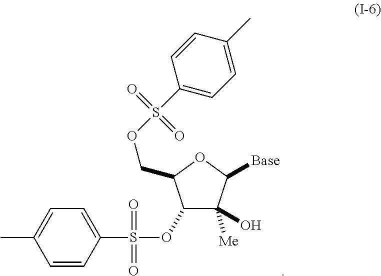

133. The process of any of embodiments 1, 2, 4 to 9, 11, 12, 14, 16, 18, 20, 21 to 54, 56, 60, 63 to 84, wherein the compound of formula (I) is a compound of formula (I-7)

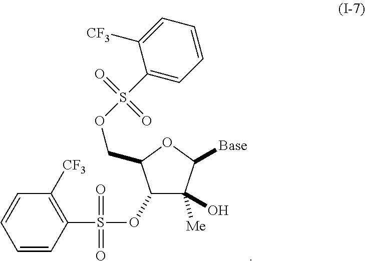

134. The process of any of embodiments 1, 2, 4 to 9, 11, 12, 14, 16, 18, 20, 21 to 54, 56, 57, 58, 61, 63 to 84 wherein the compound of formula (I) is a compound of formula (I-8)

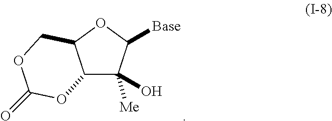

135. The process of any of embodiments 1, 2, 4 to 9, 11, 12, 14, 16, 18, 20, 21 to 54, 63 to 84 wherein the compound of formula (I) is a compound of formula (I-9)

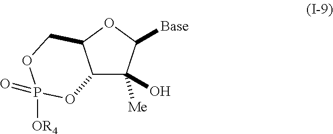

136. The process of any of embodiments 1, 2, 4 to 9, 11, 12, 14, 16, 18, 20, 21 to 54, 56, 60, 63 to 84 wherein the compound of formula (I) is a compound of formula (I-10)

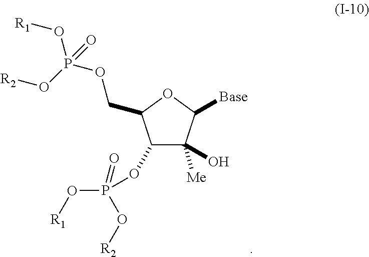

137. The process of any of embodiments 1, 2, 4 to 9, 11, 12, 14, 16, 18, 20, 21 to 54, 56, 59, 63 to 84, wherein the compound of formula (I) is a compound of formula (I-11)

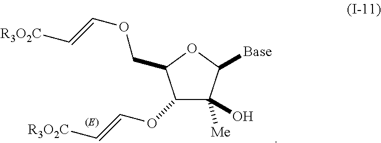

138. The process of any of embodiments 1, 2, 4 to 9, 11, 12, 14, 16, 18, 20, 21 to 54, 56, 59, 63 to 84, wherein the compound of formula (I) is a compound of formula (I-12)

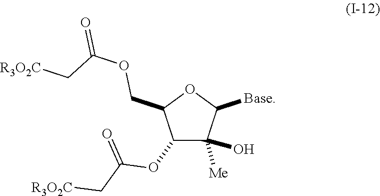

139. The process of any of embodiments 1, 2, 4 to 9, 11, 12, 14, 16, 18, 20, 21 to 54, 56, 59, 63 to 84, wherein the compound of formula (I) is a compound of formula (I-13)

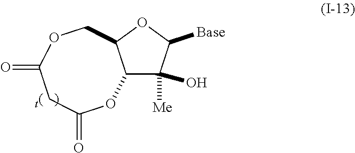

140. The process of any of embodiments 1 to 3, 10 to 13, 15, 17, 19, 21 to 52, 62 to 84, 127, wherein the compound of formula (II) is a compound of formula (II-1)

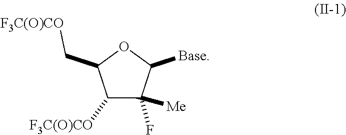

141. The process of any of embodiments 1 to 3, 10 to 13, 15, 17, 19, 21 to 52, 62 to 84, 128, wherein the compound of formula (II) is a compound of formula (II-2)

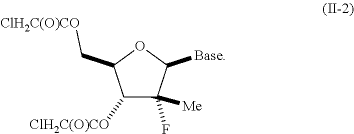

142. The process of any of embodiments 1 to 3, 10 to 13, 15, 17, 19, 21 to 52, 62 to 84, 129, wherein the compound of formula (II) is a compound of formula (II-3)

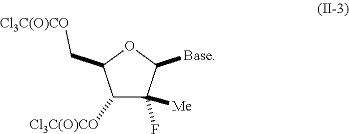

143. The process of any of embodiments 1 to 3, 10 to 13, 15, 17, 19, 21 to 52, 62 to 84, 130, wherein the compound of formula (II) is a compound of formula (II-4)

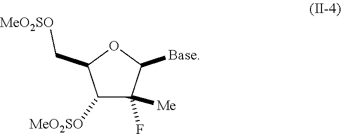

144. The process of any of embodiments 1, 2, 4 to 9, 11, 12, 14, 16, 18, 20, 21 to 52, 63 to 84, 131, wherein the compound of formula (II) is a compound of formula (II-5)

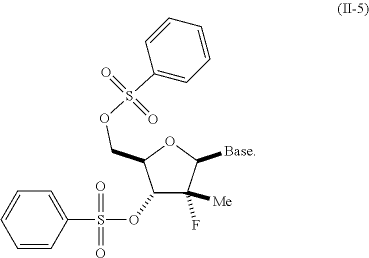

145. The process of any of embodiments 1 to 3, 11, 12, 14, 16, 18, 20, 21 to 52, 63 to 84, 132, wherein the compound of formula (II) is a compound of formula (II-6)

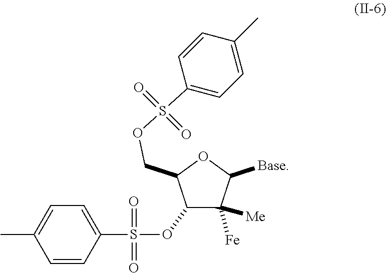

146. The process of any of embodiments 1, 2, 4 to 9, 11, 12, 14, 16, 18, 20, 21 to 52, 63 to 84, 133 wherein the compound of formula (II) is a compound of formula (II-7)

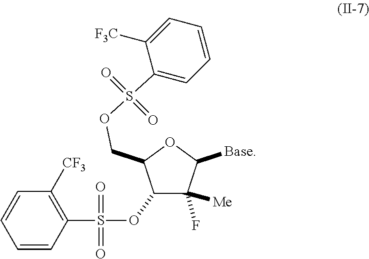

147. The process of any of embodiments 1, 2, 4 to 9, 11, 12, 14, 16, 18, 20, 21 to 52, 63 to 84, 134 wherein the compound of formula (II) is a compound of formula (II-8)

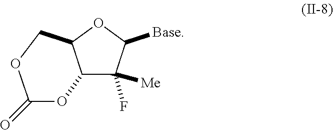

148. The process of any of embodiments 1, 2, 4 to 9, 11, 12, 14, 16, 18, 20, 21 to 52, 63 to 84, 135, wherein the compound of formula (II) is a compound of formula (II-9))

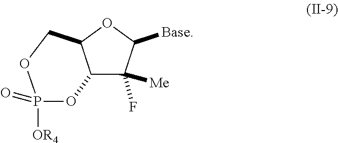

149. The process of any of embodiments 1, 2, 4 to 9, 11, 12, 14, 16, 18, 20, 21 to 52, 63 to 84, 136, wherein the compound of formula (II) is a compound of formula (II-10)

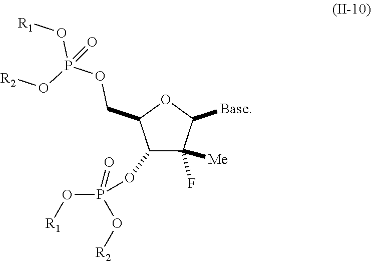

150. The process of any of embodiments 1, 2, 4 to 9, 11, 12, 14, 16, 18, 20, 21 to 52, 63 to 84, 137, wherein the compound of formula (II) is a compound of formula (II-11)

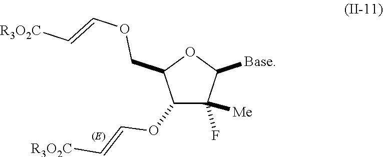

151. The process of any of embodiments 1, 2, 4 to 9, 11, 12, 14, 16, 18, 20, 21 to 52, 63 to 84, 138, wherein the compound of formula (II) is a compound of formula (II-12)

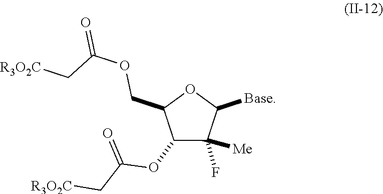

152. The process of any of embodiments 1, 2, 4 to 9, 11, 12, 14, 16, 18, 20, 21 to 52, 63 to 84, 139, wherein the compound of formula (II) is a compound of formula (II-13)

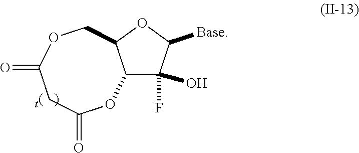

153. The process of any of embodiments 1 to 3, 10 to 13, 15, 17, 19, 21 to 55, 62 to 84, 127, 140, wherein the compound of formula (I) is a compound of formula (I-1′)

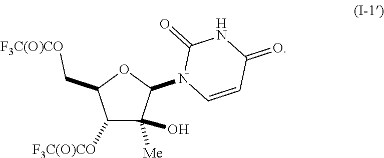

154. The process of any of embodiments 1 to 3, 10 to 13, 15, 17, 19, 21 to 55, 62 to 84, 128, 141, wherein the compound of formula (I) is a compound of formula (I-2′)

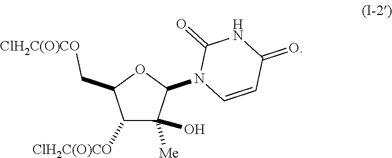

155. The process of any of embodiments 1 to 3, 10 to 13, 15, 17, 19, 21 to 55, 62 to 84, 129, 142, wherein the compound of formula (I) is a compound of formula (I-3′)

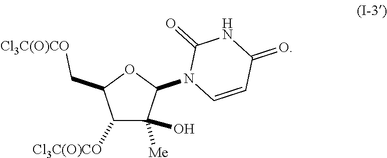

156. The process of any of embodiments 1 to 3, 10 to 13, 15, 17, 19, 21 to 55, 62 to 84, 130, 143, wherein the compound of formula (I) is a compound of formula (I-4′)

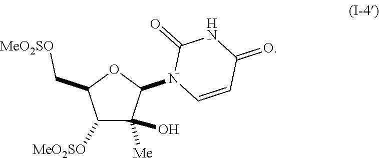

157. The process of any of embodiments 1, 2, 4 to 9, 11, 12, 14, 16, 18, 20, 21 to 54, 63 to 84, 131, 144, wherein the compound of formula (I) is a compound of formula (I-5′)

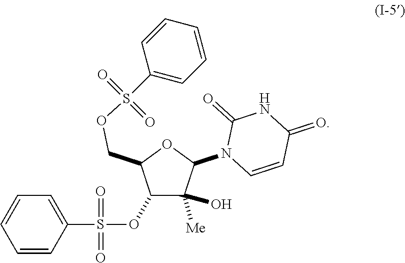 [SMILES: C[C@]1(O)[C@H](OS(=O)(=O)c2ccccc2)[C@@H](COS(=O)(=O)c2ccccc2)O[C@H]1n1ccc(=O)[nH]c1=O]

158. The process of any of embodiments 1 to 3, 11, 12, 14, 16, 18, 20, 21 to 55, 63 to 84, 132, 145 wherein the compound of formula (I) is a compound of formula (I-6′)

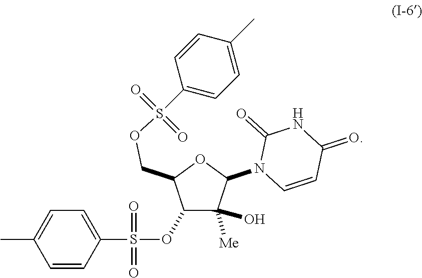 [SMILES: Cc1ccc(S(=O)(=O)OC[C@H]2O[C@@H](n3ccc(=O)[nH]c3=O)[C@@](C)(O)[C@@H]2OS(=O)(=O)c2ccc(C)cc2)cc1]

159. The process of any of embodiments 1, 2, 4 to 9, 11, 12, 14, 16, 18, 20, 21 to 54, 63 to 84, 133, 146, wherein the compound of formula (I) is a compound of formula (I-7′)

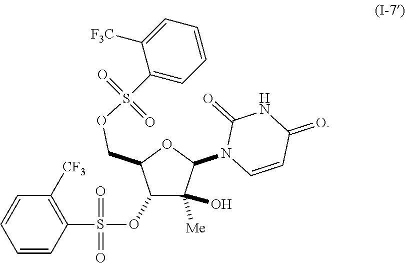 [SMILES: C[C@]1(O)[C@H](OS(=O)(=O)c2ccccc2C(F)(F)F)[C@@H](COS(=O)(=O)c2ccccc2C(F)(F)F)O[C@H]1n1ccc(=O)[nH]c1=O]

160. The process of any of embodiments 1 to 12, 14, 16, 18, 20, 21 to 54, 134, 147, wherein the compound of formula (I) is a compound of formula (I-8′)

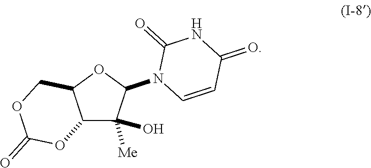

161. The process of any of embodiments 1, 2, 4 to 9, 11, 12, 14, 16, 18, 20, 21 to 54, 63 to 84, 135, 148, wherein the compound of formula (I) is a compound of formula (I-9′)

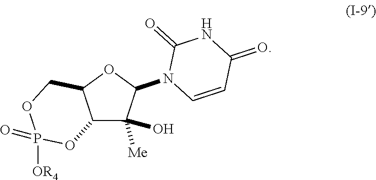

162. The process of any of embodiments 1, 2, 4 to 9, 11, 12, 14, 16, 18, 20, 21 to 54, 63 to 84, 136, 149, wherein the compound of formula (I) is a compound of formula (I-10′)

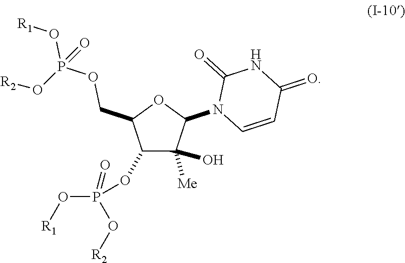

163. The process of any of embodiments 1, 2, 4 to 9, 11, 12, 14, 16, 18, 20, 21 to 54, 63 to 84, 137, 150, wherein the compound of formula (I) is a compound of formula (I-11′)

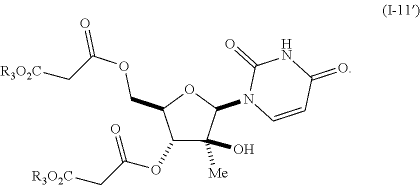

164. The process of any of embodiments 1, 2, 4 to 9, 11, 12, 14, 16, 18, 20, 21 to 54, 63 to 84, 138, 151, wherein the compound of formula (I) is a compound of formula (I-12′)

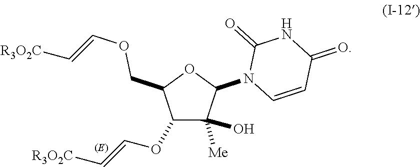

165. The process of any of embodiments 1, 2, 4 to 9, 11, 12, 14, 16, 18, 20, 21 to 54, 63 to 84, 139, 152, wherein the compound of formula (I) is a compound of formula (I-13′)

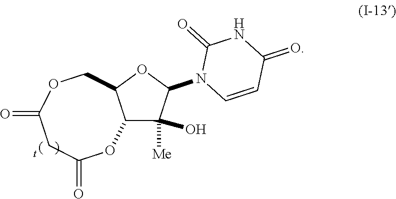

166. The process of any of embodiments 1 to 3, 10 to 13, 15, 17, 19, 21 to 54, 62 to 84, 127, 140, 153, wherein the compound of formula (II) is a compound of formula (II-1′)

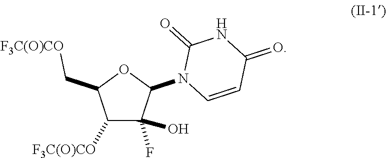

167. The process of any of embodiments 1 to 3, 10 to 13, 15, 17, 19, 21 to 52, 62 to 84, 128, 141, 154 wherein the compound of formula (II) is a compound of formula (II-2′)

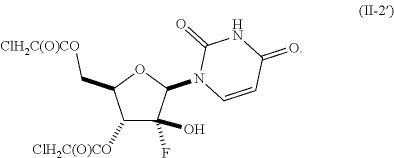

168. The process of any of embodiments 1 to 3, 10 to 13, 15, 17, 19, 21 to 52, 62 to 84, 129, 142, 155, wherein the compound of formula (II) is a compound of formula (II-3′)

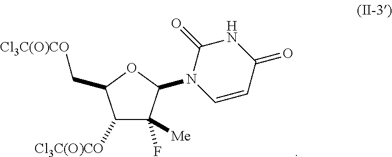

169. The process of any of embodiments 1 to 3, 10 to 13, 15, 17, 19, 21 to 52, 62 to 84, 130, 143, 156, wherein the compound of formula (II) is a compound of formula (II-4′)

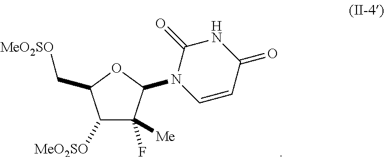

170. The process of any of embodiments 1 to 3, 11, 12, 14, 16, 18, 20, 21 to 52, 63 to 83, 131, 144, 157, wherein the compound of formula (II) is a compound of formula (II-5′)

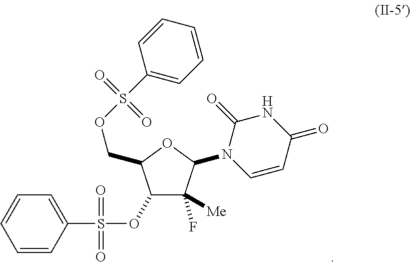 [SMILES: C[C@@]1(F)[C@H](OS(=O)(=O)c2ccccc2)[C@@H](COS(=O)(=O)c2ccccc2)O[C@H]1n1ccc(=O)[nH]c1=O]

171. The process of any of embodiments 1 to 3, 11, 12, 14, 16, 18, 20, 21 to 52, 63 to 84, 132, 145, 158, wherein the compound of formula (II) is a compound of formula (II-6′)

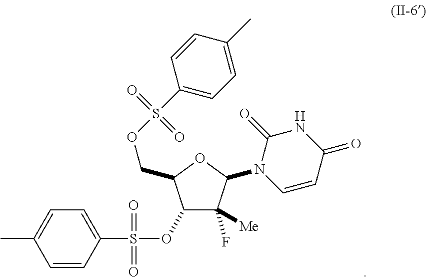 [SMILES: Cc1ccc(S(=O)(=O)OC[C@H]2O[C@@H](n3ccc(=O)[nH]c3=O)[C@](C)(F)[C@@H]2OS(=O)(=O)c2ccc(C)cc2)cc1]

172. The process of any of embodiments 1, 2, 4 to 9, 11, 12, 14, 16, 18, 20, 21 to 52, 63 to 84, 133, 146, 159, wherein the compound of formula (II) is a compound of formula (II-7′)

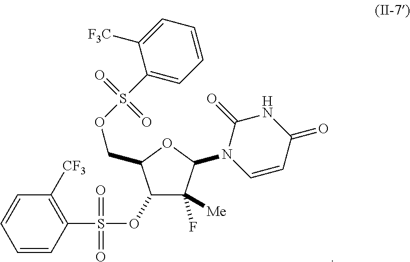 [SMILES: C[C@@]1(F)[C@H](OS(=O)(=O)c2ccccc2C(F)(F)F)[C@@H](COS(=O)(=O)c2ccccc2C(F)(F)F)O[C@H]1n1ccc(=O)[nH]c1=O]

173. The process of any of embodiments 1, 2, 4 to 9, 11, 12, 14, 16, 18, 20, 21 to 52, 63 to 84, 134, 147, 160, wherein the compound of formula (II) is a compound of formula (II-8′)

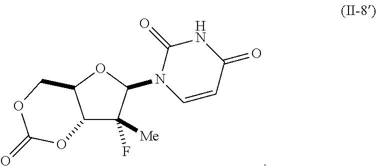

174. The process of any of embodiments 1, 2, 4 to 9, 11, 12, 14, 16, 18, 20, 21 to 52, 63 to 84, 135, 148, 161, wherein the compound of formula (II) is a compound of formula (II-9′)

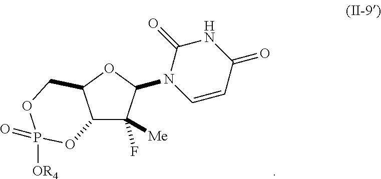

175. The process of any of embodiments 1, 2, 4 to 9, 11, 12, 14, 16, 18, 20, 21 to 52, 63 to 84, 136, 149, 162, wherein the compound of formula (II) is a compound of formula (II-10′)

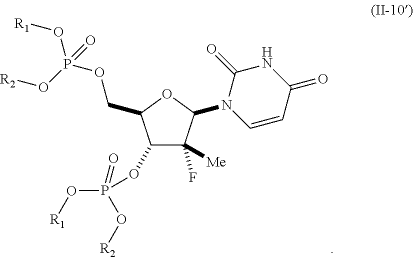

176. The process of any of embodiments 1, 2, 4 to 9, 11, 12, 14, 16, 18, 20, 21 to 52, 63 to 84, 137, 150, 163, wherein the compound of formula (II) is a compound of formula (II-11′)

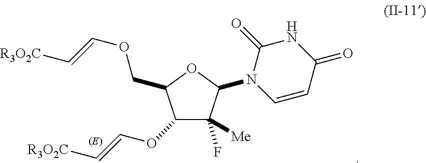

177. The process of any of embodiments 1, 2, 4 to 9, 11, 12, 14, 16, 18, 20, 21 to 52, 63 to 84, 138, 151, 164, wherein the compound of formula (II) is a compound of formula (II-12′)

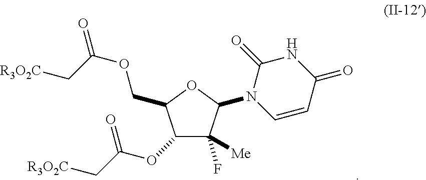

178. The process of any of embodiments 1, 2, 4 to 9, 11, 12, 14, 16, 18, 20, 21 to 52, 63 to 84, 139, 152, 165, wherein the compound of formula (II) is a compound of formula (II-13′)

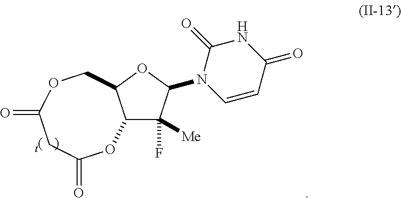

The present invention is further illustrated by the following examples and comparative examples.

### EXAMPLES

### Abbreviation List

Ac acetate
Bz benzoyl
DAST diethylaminosulfur trifluoride
DCM dichloromethane
DBU 1,8-Diazabicyclo[5.4.0]undec-7-ene
DMAP 4-dimethylamino pyridine
Et ethyl
EtOAc ethyl acetate
HPLC high pressure liquid chromatography
M molar, molarity
Me methyl
mM millimolar
MeOH methanol
Ms mesyl, methanesulfonyl
NMR nuclear magnetic resonance
r.t. room temperature
TEA triethylamine
THF tetrahydrofuran
TLC thin layer chromatography
TMS tetramethylsilane
UV ultraviolet
XTalFluor E (Diethylamino)difluorosulfonium tetrafluoroborate

General Analytical Methods

Reactions were monitored by HPLC on a C-18 reverse phase column with a gradient of acetonitrile in 10 mM ammonium sulfamate aqueous buffer at pH 5.6 or 40 mM aqueous sulfamic acid, or using thin layer chromatography (TLC) on silica gel pre-coated aluminum sheets (Silica gel 60 F₂₅₄, Merck). TLC visualization was accomplished by irradiation with UV light at 254 nm and/or a ceric ammonium molybdate stain. ¹H and ¹³C chemical shifts are reported in ppm relative to TMS (0 ppm) with the solvent resonance as the internal standard (CDCl₃, ¹H: 7.26 ppm, ¹³C: 77.16 ppm, (CD₃)₂O ¹H: 2.05 ppm, ¹³C: 29.84, 202.26 ppm).

### Example 1: Preparation of 2′-C-methyl arabino-uridine

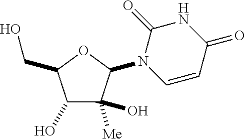 [SMILES: C[C@]1(O)[C@H](O)[C@@H](CO)O[C@H]1n1ccc(=O)[nH]c1=O]

The preparation of 2′-C-methyl arabino-uridine is described in (a) Tetrahedron Lett. 2007, 48, 4441; (b) Nucleosides, Nucleotides and Nucleic Acids, 2011, 30, 886 and (c) Chemical and Pharmaceutical Bulletin, 1988, 36, 945. 2′-C-methyl arabino-uridine was prepared according to reference (b) using non-deuterated MeMgBr.

### Example 2: Synthesis of (2R,3R,4S,5R)-5-(2,4-dioxo-3,4-dihydropyrimidin-1(2H)-yl)-4-hydroxy-4-methyl-2-((2,2,2-trichloroacetoxy)methyl)tetrahydrofuran-3-yl 2,2,2-trichloroacetate

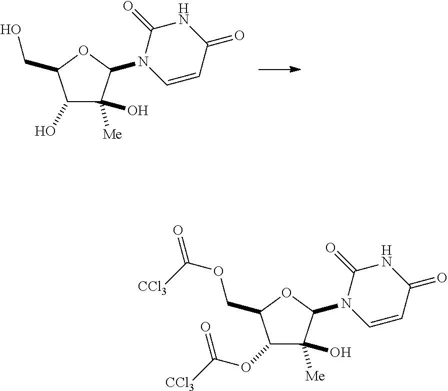

In a two-neck round bottom flask purged with nitrogen, 2′-C-methyl arabino-uridine prepared according to Example 1 (500 mg, 1.94 mmol, 1 equiv) was suspended in anhydrous DCM (10 mL). To this suspension was added pyridine (3.13 mL, 38.7 mmol, 20 equiv), DMAP (118 mg, 0.97 mmol, 0.5 equiv) and trichloroacetyl chloride (648 microL, 5.8 mmol, 3 equiv). The addition of trichloroacetyl chloride is highly exothermic. Hence, it was added dropwise. The homogeneous mixture was stirred at r.t. for 17 hours, and diluted with DCM (50 mL). The crude reaction mixture was washed with 2.5M HCl (50 mL×3), sat. aq. NaHCO₃ (50 mL×2), and the organic phase was dried over Na₂SO₄. Evaporation of volatiles under reduced pressure gave the title compound as a light orange foam (885 mg, 1.61 mmol, 83%) in spectroscopically pure form. The foam was further recrystallized from diethyl ether/pentane to give an off-white fine powder. Characterization data for the product:

¹H NMR (300 MHz, (CD₃)₂O): 10.14 (br s, 1H), 7.78 (d, J=8.2 Hz, 1H), 6.16 (s, 1H), 5.60 (d, J=8.2 Hz, 1H), 5.34 (d, J=2.4 Hz, 1H), 4.99 (dd, J=7.2 Hz, 11.6 Hz, 1H), 4.83 (dd, J=4.0 Hz, 11.6 Hz), 4.61-4.54 (m, 1H), 1.48 (s, 3H).

¹³C NMR (75 MHz, (CD₃)₂O): 163.6, 162.4, 161.5, 151.7, 142.9, 101.5, 90.4, 90.1, 89.3, 84.6, 80.0, 79.3, 69.1, 19.1.

### Example 3: Synthesis of (2R,3R,4S,5R)-5-(2,4-dioxo-3,4-dihydropyrimidin-1(2H)-yl)-4-hydroxy-4-methyl-2-((2,2,2-trifluoroacetoxy)methyl)tetrahydrofuran-3-yl 2,2,2-trifluoroacetate

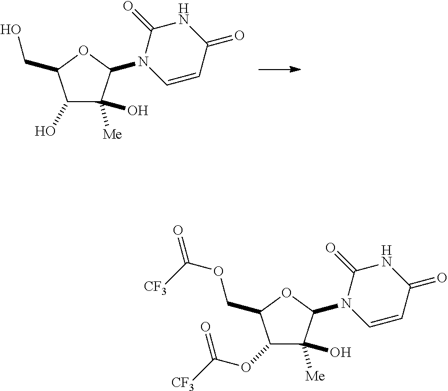 [SMILES: C[C@]1(O)[C@H](O)[C@@H](CO)O[C@H]1n1ccc(=O)[nH]c1=O ; C[C@]1(O)[C@H](OC(=O)C(F)(F)F)[C@@H](COC(=O)C(F)(F)F)O[C@H]1n1ccc(=O)[nH]c1=O]

In a two-neck round bottom flask purged with nitrogen, 2′-C-methyl arabino-uridine prepared according to Example 1 (100 mg, 0.387 mmol, 1 equiv) was suspended in anhydrous DCM (2 mL). To this suspension was added trifluoroacetic anhydride (109 microL, 0.775 mmol, 2 equiv), whereby starting material dissolution was observed within several minutes. The mixture was stirred at r.t. for 2.5 hours and evaporated to give the crude compound (200 mg). Evaporation of volatiles under high vacuum overnight furnished the title as a white solid in spectroscopically pure form and did not require further purification (157 mg, 0.349 mmol, 90%). Characterization data for the product:

¹H NMR (300 MHz, (CD₃)₂O): 10.28 (br s, 1H), 7.77 (d, J=8.2 Hz, 1H), 6.14 (s, 1H), 5.62 (d, J=8.2 Hz, 1H), 5.34 (d, J=2.3 Hz, 1H), 4.96 (dd, J=7.0 Hz, 11.7 Hz, 1H), 4.84 (dd, J=4.0 Hz, 11.7 Hz), 4.65-4.59 (m, 1H), 1.44 (s, 3H).

¹³C NMR (75 MHz, (CD₃)₂O): 164.1, 157.8 (q, J=43 Hz), 157.0 (q, J=43 Hz), 151.6, 143.2, 115.5 (q, J=285 Hz), 115.4 (q, J=285 Hz), 101.4, 89.2, 83.8, 79.9, 79.0, 67.9, 18.8.

### Example 4: Synthesis of ((2R,3R,4S,5R)-3-(2-chloroacetoxy)-5-(2,4-dioxo-3,4-dihydropyrimidin-1(2H)-yl)-4-hydroxy-4-methyltetrahydrofuran-2-yl)methyl 2-chloroacetate

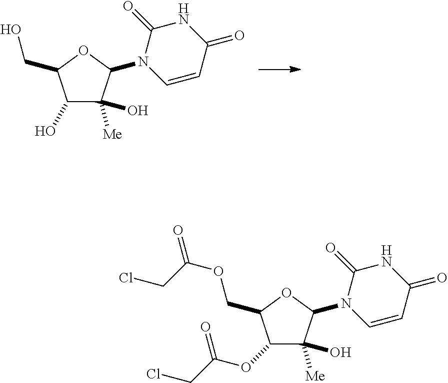 [SMILES: C[C@]1(O)[C@H](O)[C@@H](CO)O[C@H]1n1ccc(=O)[nH]c1=O ; C[C@]1(O)[C@H](OC(=O)CCl)[C@@H](COC(=O)CCl)O[C@H]1n1ccc(=O)[nH]c1=O]

In a two-neck round bottom flask purged with nitrogen, 2′-C-methyl arabino-uridine prepared according to Example 1 (200 mg, 0.77 mmol, 1 equiv) was dissolved in pyridine (4 mL) and the solution was cooled to 0° C. Chloroacetic anhydride (331 mg, 1.94 mmol, 2.5 equiv) was added. The clear dark-brown reaction mixture was removed from the ice bath and stirred for 2 h at r.t. The crude reaction mixture was cooled to r.t. in an ice bath, whereby white precipitate formed. The reaction was diluted with EtOAc (40 mL) and the organic phase was washed with 2.5M HCl (100 mL×3) and sat. aq. NaHCO₃ (100 mL×3). The combined aqueous phases were washed with EtOAc (50 mL) and the combined organic phases were dried over Na₂SO₄ and evaporated. The title product was obtained spectroscopically pure form as a colorless oil (106 mg, 0.256 mmol, 33%). Characterization data for the product:

¹H NMR (300 MHz, CDCl₃): 10.40 (br s, 1H), 7.66 (d, J=8.2 Hz, 1H), 6.01 (s, 1H), 5.62 (d, J=8.2 Hz, 1H), 5.01 (d, J=2.3 Hz, 1H), 4.64 (dd, J=6.8 Hz, 11.7 Hz, 1H), 4.48 (dd, J=4.1 Hz, 11.7 Hz), 4.24-4.12 (m, 5H), 1.42 (s, 3H).

¹³C NMR (75 MHz, CDCl₃): 167.3, 166.5, 164.6, 150.9, 142.9, 101.3, 89.1, 80.8, 80.5, 78.8, 64.6, 40.8, 40.5, 18.95.

### Example 5: Synthesis of (2R,3R,4S,5R)-5-(2,4-dioxo-3,4-dihydropyrimidin-1(2H)-yl)-4-hydroxy-4-methyl-2-(((methylsulfonyl)oxy)methyl)tetrahydrofuran-3-yl methanesulfonate

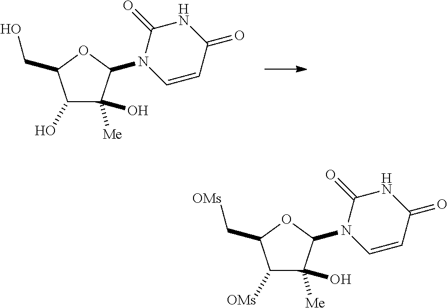 [SMILES: C[C@]1(O)[C@H](O)[C@@H](CO)O[C@H]1n1ccc(=O)[nH]c1=O ; C[C@]1(O)[C@H](OS(C)(=O)=O)[C@@H](COS(C)(=O)=O)O[C@H]1n1ccc(=O)[nH]c1=O]

In a two-neck round bottom flask purged with nitrogen, 2′-C-methyl arabino-uridine prepared according to Example 1 (300 mg, 1.16 mmol, 1 equiv) was suspended in dichloromethane (12 mL). To the suspension was added 2,6-lutidine (1.08 mL, 9.29 mmol, 8 equiv), methanesulfonyl chloride (540 microL, 7.02 mmol, 6 equiv) and DMAP (14.2 mg, 0.12 mmol, 0.1 equiv). The homogeneous mixture was stirred for 15 h, after which all of the starting material was consumed. Volatiles were removed under reduced pressure, the crude reaction was taken up in THF (5 mL) and the organic phase was washed with a mixture of 2.5M HCl (2 mL) and sat. aq. NaCl (2 mL) three times, followed by sat. aq. NaCl (5 mL) once. The organic phase was dried over Na₂SO₄, evaporated and dried under vacuum at 35° C. for 17 h. The title product was obtained spectroscopically pure form as an off-white solid (409 mg, 0.95 mmol, 82%). Characterization data for the product:

¹H NMR (300 MHz, (CD₃)₂O): 10.12 (br s, 1H), 7.77 (d, J=8.2 Hz, 1H), 6.08 (s, 1H), 5.59 (d, J=8.2 Hz, 1H), 4.90 (d, J=2.8 Hz, 1H), 4.67-4.62 (m, 2H), 4.54-4.47 (m, 1H), 3.33 (s, 3H), 3.21 (s, 3H), 1.47 (s, 3H).

### Example 6: Synthesis of (2R,3R,4S,5R)-5-(2,4-dioxo-3,4-dihydropyrimidin-1(2H)-yl)-4-hydroxy-4-methyl-2-((pivaloyloxy)methyl)tetrahydrofuran-3-yl pivalate

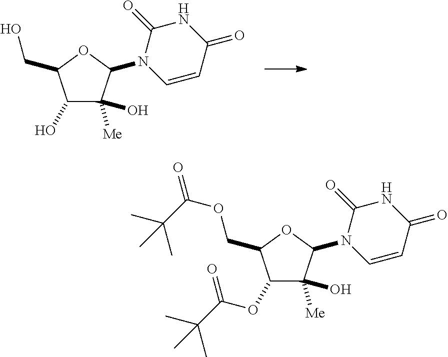 [SMILES: C[C@]1(O)[C@H](O)[C@@H](CO)O[C@H]1n1ccc(=O)[nH]c1=O ; CC(C)(C)C(=O)OC[C@H]1O[C@@H](n2ccc(=O)[nH]c2=O)[C@@](C)(O)[C@@H]1OC(=O)C(C)(C)C]

In a two-neck round bottom flask purged with nitrogen, 2′-C-methyl arabino-uridine prepared according to Example 1 (2.0 g, 7.75 mmol, 1 equiv) was dissolved in pyridine (40 mL) and the light-orange solution was cooled to 0° C. Pivaloyl chloride (3.05 mL, 24.8 mmol, 3.2 equiv) was added, whereby a white precipitate was observed. The reaction mixture was stirred for 10 min at 0° C., then 4 h at 50° C. and overnight at 80° C. The crude reaction mixture was cooled to r.t. in an ice bath, whereby white precipitate formed. The precipitate was filtered off and discarded. To the filtrate was added EtOAc (100 mL) and the organic phase was washed with 2.5M HCl (100 mL×3), sat. aq. NaHCO₃ (100 mL×1), dried over Na₂SO₄ and evaporated. The crude product was passed through a pad of silica washing with EtOAc to give the title product in spectroscopically pure form as an off-white foam (2.72 g, 6.38 mmol, 82%). Characterization data for the product:

¹H NMR (300 MHz, CDCl₃): 9.67 (br s, 1H), 7.67 (d, J=8.2 Hz, 1H), 6.03 (s, 1H), 5.61 (dd, J=1.7 Hz, 8.2 Hz, 1H), 4.88 (d, J=2.8 Hz, 1H), 4.59 (dd, J=7.1 Hz, 11.8 Hz, 1H), 4.30 (dd, J=3.9 Hz, 11.8 Hz), 4.14-4.05 (m, 1H), 1.26 (s, 3H), 1.24 (s, 9H), 1.22 (s, 9H).

¹³C NMR (75 MHz, CDCl₃): 178.4, 177.6, 164.5, 150.9, 142.9, 101.3, 89.6, 81.4, 79.7, 79.2, 63.5, 39.0, 38.9, 27.3, 27.2, 19.4.

### Example 7: Synthesis of ((2R,3R,4S,5R)-3-(benzoyloxy)-5-(2,4-dioxo-3,4-dihydropyrimidin-1(2H)-yl)-4-hydroxy-4-methyltetrahydrofuran-2-yl)methyl benzoate

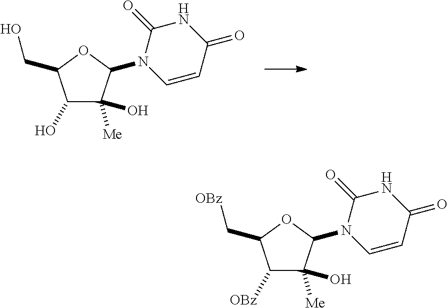

In a two-neck round bottom flask purged with nitrogen, 2′-C-methyl arabino-uridine prepared according to Example 1 (5.00 g, 19.5 mmol, 1 equiv) was dissolved in pyridine (100 mL) and the solution was cooled to 0° C. Benzoyl chloride (6.4 mL, 55.1 mmol, 2.82 equiv) was added. The reaction mixture was removed from the ice bath and stirred for 19.5 h at r.t. The crude reaction mixture was cooled to r.t. in an ice bath, whereby white precipitate formed. The reaction was diluted with EtOAc (250 mL) and the organic phase was washed with 2.5M HCl (250 mL×3) and sat. aq. NaHCO₃ (250 mL), whereby the precipitate dissolved. The organic phase was dried over Na₂SO₄, evaporated and dry-loaded onto a silica gel column packed with EtOAc/heptane (1:1). Elution with EtOAc/heptane gradient (1:1 to 7:3) afforded the title compound as a white solid (5.4 g, 11.6 mmol, 59%). Characterization data for the product:

¹H NMR (300 MHz, CDCl₃): 9.55 (br s, 1H), 8.12-8.00 (m, 4H), 7.74 (d, J=8.2 Hz, 1H), 7.64-7.53 (m, 2H), 7.50-7.40 (m, 4H), 6.18 (s, 1H), 5.55 (br d, J=8.2 Hz, 1H), 5.30 (d, J=3.3 Hz, 1H), 4.85 (dd, J=6.6 Hz, 12.0 Hz, 1H), 4.75 (dd, J=3.9 Hz, 12.0 Hz), 4.48-4.40 (m, 1H), 1.54 (s, 3H).

¹³C NMR (75 MHz, CDCl₃): 166.5, 165.8, 164.7, 151.0, 143.1, 134.0, 133.5, 129.96, 129.89, 129.7, 128.84, 128.79, 128.6, 101.3, 89.4, 81.2, 80.5, 79.2, 64.1, 19.7.

### Fluorination of the 3′,5′-protected nucleosides

### Example 8: Preparation of 2′-deoxy-2′-fluoro-2′-C-methyl uridine via fluorination of (2R,3R,4S,5R)-5-(2,4-dioxo-3,4-dihydropyrimidin-1(2H)-yl)-4-hydroxy-4-methyl-2-((2,2,2-trifluoroacetoxy)methyl)tetrahydrofuran-3-yl 2,2,2-trifluoroacetate with XTalFluor E and deprotection

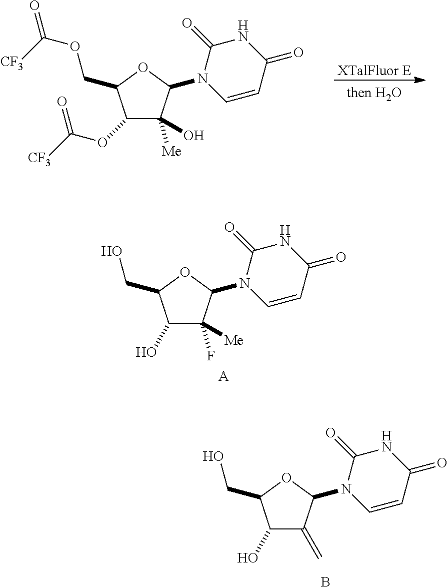

In a two-neck round bottom flask purged with nitrogen, triethylamine (37 microL, 0.26 mmol, 1 equiv) was dissolved in anhydrous DCM (3 mL) and TEA.3HF (86 microL, 0.53 mmol, 2 equiv) was added at room temperature. To this solution, XTalFluor E (91 mg, 0.40 mmol, 1.5 equiv) was added at r.t., followed by 3′,5′-di-O-trifluoroacetyl-2′-C-methyl arabino-uridine prepared according to Example 3 (119 mg, 0.26 mmol, 1 equiv) in DCM (0.5 mL, partially soluble). The homogeneous, light brown reaction mixture was stirred at r.t. for 4 h and evaporated. The crude mixture was dissolved in D₂O (results in loss oftrifluoroacetyl group within seconds) and filtered. NMR spectroscopy indicated a mixture of 2′-deoxy-2′-fluoro-2′-C-methyl uridine A (42%), the unsaturated compound B (37%), 2′-C-methyl arabino-uridine (8%) and an unknown side product (13%). Characterization data for the products:

### Compound A

¹H NMR (300 MHz, (CD₃)₂O): 10.23 (br s, 1H), 8.12 (d, J=8.2 Hz, 1H), 6.13 (d, J=18.6 Hz, 1H), 5.62 (d, J=8.2 Hz, 1H), 4.19-3.80 (m, 4H), 1.39 (d, J=22.3 Hz, 3H).

¹H NMR (300 MHz, (D₂O): 7.74 (d, J=8.2 Hz, 1H), 6.10 (d, J=19.4 Hz, 1H), 5.81 (d, J=8.2 Hz, 1H), 4.02-3.85 (m, 3H), 3.79-3.71 (m, 1H), 1.29 (d, J=23.3 Hz, 3H).

¹³C NMR (75 MHz, (D₂O): 165.9, 151.5, 141.2, 102.6, 100.9 (d, J=179.7 Hz), 89.4 (d, J=39.2 Hz), 81.2, 71.2 (d, J=17.9 Hz), 59.0, 15.8 (d, J=25.2 Hz).

### Compound B

¹H NMR (300 MHz, (CD₃)₂O): 10.18 (br s, 1H), 7.62 (d, J=8.1 Hz, 1H), 6.60 (br s, 1H), 5.63 (d, J=8.1 Hz, 1H), 5.51 (apparent t, J=2.0 Hz, 1H), 5.38 (apparent t, J=2.0 Hz, 1H), 4.88 (br s, 1H), 4.76 (br d, J=4.9 Hz, 1H), 3.95-3.74 (m, 3H).

¹³C NMR (75 MHz, (CD₃)₂O): 163.8, 151.7, 151.1, 142.2, 112.8, 103.0, 86.0, 85.0, 71.1, 61.6.

### Example 9: Fluorination of ((2R,3R,4S,5R)-3-(2-chloroacetoxy)-5-(2,4-dioxo-3,4-dihydropyrimidin-1(2H)-yl)-4-hydroxy-4-methyltetrahydrofuran-2-yl)methyl 2-chloroacetate with XTAlFluor E

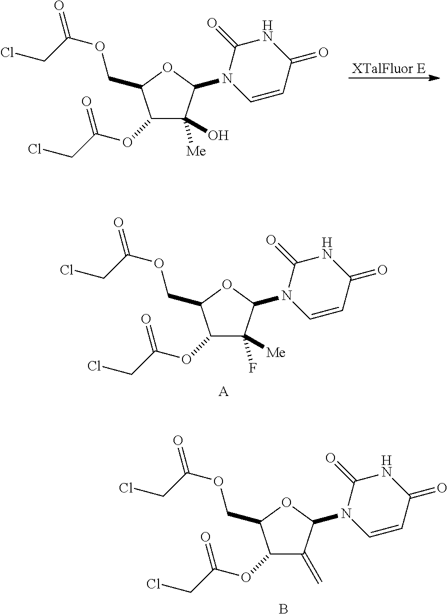

In a two-neck round bottom flask purged with nitrogen, triethylamine (17 microL, 0.12 mmol, 1 equiv) was dissolved in anhydrous DCM (1 mL) and TEA.3HF (40 microL, 0.24 mmol, 2 equiv) was added at room temperature. To this solution, XTalFluor E (42 mg, 0.18 mmol, 1.5 equiv) was added at r.t., followed by 3′,5′-di-O-chloroacetyl-2′-C-methyl arabino-uridine prepared according to Example 4 (50 mg, 0.12 mmol, 1 equiv) in DCM (0.5 mL). The homogeneous reaction mixture was stirred at r.t. for 50 min, after which a fresh portion of XTalFluor E (28 mg, 0.12 mmol, 1 equiv) was added and the reaction was stirred for a further 2 h. The reaction mixture was diluted with DCM (50 mL), and the organic phase was washed with sat. aq. NaHCO₃ (100 mL×3) and sat. aq. NaCl (100 mL×1), dried over Na₂SO₄ and evaporated. NMR spectroscopy indicated a mixture of the title compound A (47%), the unsaturated compound B (24%), 2′-C-methyl arabino-uridine (8%) and an unknown side product (29%). Characterization data for product A:

¹H NMR (300 MHz, (CD₃)₂O): 10.35 (br s, 1H), 7.78 (d, J=8.2 Hz, 1H), 6.12 (br d, J=19.9 Hz, 1H), 5.75 (d, J=8.2 Hz, 1H), 5.63 (s, 1H), 5.50 (br d, J=12.5 Hz, 1H), 4.63-4.36 (m, 6H), 1.48 (d, J=22.8 Hz, 3H).

¹³C NMR (75 MHz, CDCl₃): 167.8, 167.7, 163.2, 151.3, 141.3, 103.6, 100.9 (d, J=184.3 Hz), 91.4 (br), 78.0, 74.4 (d, J=15.9 Hz), 64.2, 41.50, 41.48, 17.6 (d, J=24.7 Hz).

### Example 10: Preparation of 2′-deoxy-2′-fluoro-2′-C-methyl uridine via fluorination of (2R,3R,4S,5R)-5-(2,4-dioxo-3,4-dihydropyrimidin-1(2H)-yl)-4-hydroxy-4-methyl-2-((2,2,2-trichloroacetoxy)methyl)tetrahydrofuran-3-yl 2,2,2-trichloroacetate with XTalFluor followed by deprotection

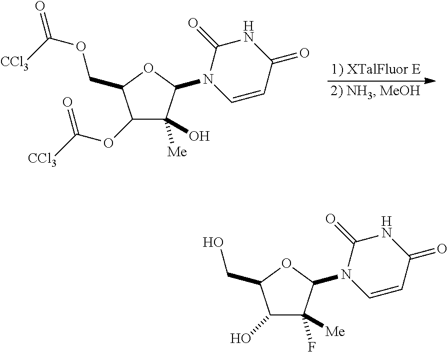

### 2′-deoxy-2′-fluoro-2′-C-methyl uridine

In a two-neck round bottom flask purged with nitrogen, 3′,5′-di-O-trichloroacetyl-2′-C-methyl arabino-uridine prepared according to Example 2 (634.5 mg, 1.156 mmol, 1 equiv) was dissolved in DCM (15 mL). To this solution XTalFluor E (450 mg, 1.965 mmol, 1.7 equiv) was added at r.t, followed by a 0.36 M TEA.2HF solution in DCM¹ Prepared the following way: in a 10 mL graduated cylinder filled with ca. 5 mL DCM, 400 μL TEA.3HF (2 equiv, Aldrich) was added, followed by triethylamine (171 microL, 1 equiv). The graduated cylinder was filled to the 10 mL mark and shaken. This solution is hygroscopic and should be used within a day. (4.765 mL, 1.734 mmol, 1.5 equiv). The homogeneous reaction mixture was stirred at r.t. for 1 h, after which in-process control indicated full consumption of the starting material. The crude mixture was diluted with DCM (35 mL), extracted with a pH 7 buffer (14 mL×3), dried over Na₂SO₄ and evaporated. The yield of the reaction was determined to be 58% by ¹H NMR. The crude reaction mixture was dissolved MeOH (23 mL), charged with methanolic ammonium (7M, 826 microL, 5.78 mmol) and stirred at r.t. for 45 min, at which point in-process control indicated full conversion of the starting material. The crude reaction mixture was evaporated to dryness, and dry-loaded onto a silica gel column packed with cyclohexane. Elution with EtOAc afforded the title compound as a white solid (139 mg, 0.53 mmol, 46% over 2 steps). Characterization data for the product:

¹H NMR (300 MHz, D₂O): 7.74 (d, J=8.2 Hz, 1H), 6.10 (d, J=19.4 Hz, 1H), 5.81 (d, J=8.2 Hz, 1H), 4.02-3.85 (m, 3H), 3.79-3.71 (m, 1H), 1.29 (d, J=23.3 Hz, 3H).

¹³C NMR (75 MHz, D₂O): 165.9, 151.5, 141.2, 102.6, 100.9 (d, J=179.7 Hz), 89.4 (d, J=39.2 Hz), 81.2, 71.2 (d, J=17.9 Hz), 59.0, 15.8 (d, J=25.2 Hz).

### Example 11: Fluorination of ((2R,3R,4S,5R)-5-(2,4-dioxo-3,4-dihydropyrimidin-1(2H)-yl)-4-hydroxy-4-methyl-2-(((methylsulfonyl)oxy)methyl)tetrahydrofuran-3-yl methanesulfonate with XTalFluor E

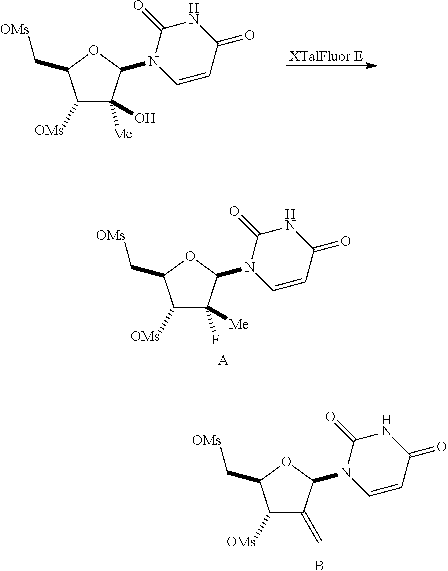

In a two-neck round bottom flask purged with nitrogen, 3′,5′-di-O-methanesulfonyl-2′-C-methyl arabino-uridine prepared according to Example 5 (61 mg, 0.14 mmol, 1 equiv) was dissolved in a mixture of DCM (0.5 mL) and THF (1 mL). To this solution XTalFluor E (55 mg, 0.24 mmol, 1.7 equiv) was added at r.t, followed by a 0.37 M TEA.2HF solution in DCM (0.58 mL, 0.21 mmol, 1.5 equiv). The homogeneous reaction mixture was stirred at r.t. for 1.5 h, after which in-process control indicated incomplete consumption of the starting material. XTalFluor E (10 mg, 0.04 mmol, 0.3 equiv) was added and the reaction was stirred for an additional 50 min, after which all of the starting material was consumed. Volatiles were removed under reduced pressure, the crude reaction was taken up in THF (5 mL) and the organic phase was washed with a mixture of sat. aq. NaCl (1 mL) and aq. pH7 buffer (1 mL) three times, dried over Na₂SO₄ and evaporated. NMR spectroscopy indicated a mixture of the title compound A (26%), the unsaturated compound B (63%), and an unknown side product (21%). Characterization data for product A:

¹H NMR (300 MHz, (CD₃)₂O): 10.40 (br s, 1H), 7.76 (d, J=8.1 Hz, 1H), 6.11 (br d, J=19.6 Hz, 1H), 5.69 (d, J=8.1 Hz, 1H), 5.32 (br d, J=18.6 Hz, 1H), 4.79-4.58 (m, 2H), 4.53-4.41 (m, 1H), 3.40 (s, 3H), 3.21 (s, 3H), 1.58 (d, J=22.9 Hz, 3H).

### Comparative Example 1: Fluorination of (2R,3R,4S,5R)-5-(2,4-dioxo-3,4-dihydropyrimidin-1(2H)-yl)-4-hydroxy-4-methyl-2-((pivaloyloxy)methyl)tetrahydrofuran-3-yl pivalate with XTalFluor E

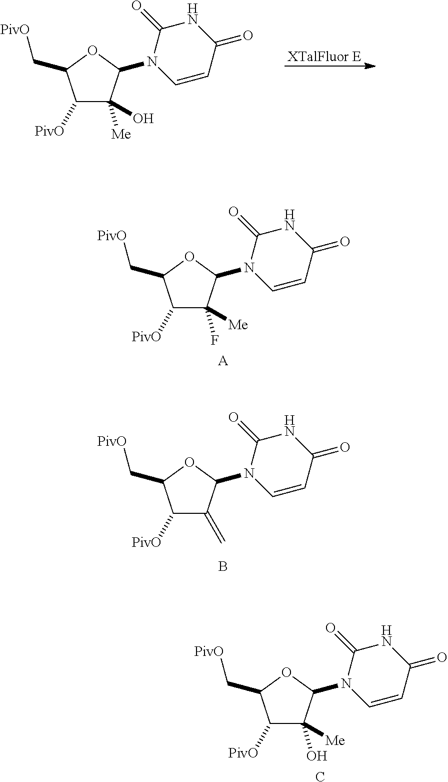

In a two-neck round bottom flask purged with nitrogen, triethylamine (163 μL, 1.17 mmol, 1 equiv) was dissolved in anhydrous DCM (7.5 mL) and TEA.3HF (385 μL, 2.36 mmol, 2 equiv) was added at room temperature. To this solution, XTalFluor E (405 mg, 1.77 mmol, 1.5 equiv) was added at r.t., followed by 3′,5′-di-O-pivaloyl-2′-C-methyl arabino-uridine (500 mg, 1.17 mmol, 1 equiv) prepared according to Example 6. The reaction mixture was stirred at r.t. for 1 h and dissolved in DCM (15 mL). The organic phase was washed with sat. aq. NaHCO₃ (100 mL×3) and sat. aq. NH₄Cl (100 mL×1), dried over Na₂SO₄ and evaporated. Purification by flash column chromatography eluting with a gradient of EtOAc/heptane (1:2 to 2:1) afforded the title product A as white solid (82.9 mg, 0.21 mmol, 18%) along with the unsaturated product B (32.1 mg, 0.078 mmol, 7%), the starting material epimer C (158.4 mg, 0.37 mmol, 32%) and an unknown side product (82.7 mg).

Characterization Data for the Products:

Product A: ¹H NMR (300 MHz, CDCl₃): 8.82 (br s, 1H), 7.59 (d, J=8.2 Hz, 1H), 6.23 (d, J=18.4 Hz, 1H), 5.78 (d, J=8.2 Hz, 1H), 5.06 (dd, J=9.4, 21.5 Hz, 1H), 4.46-4.29 (m, 3H), 1.35 (d, J=22.2 Hz, 3H), 1.26 (s, 9H), 1.24 (s, 9H).

¹³C NMR (75 MHz, CDCl₃): 177.7, 177.4, 162.9, 150.2, 138.8, 103.1, 99.4 (d, J=187.0 Hz), 89.6 (d, J=40.6 Hz), 77.2, 70.7 (d, J=16.6 Hz), 61.3, 39.0, 38.9, 27.1, 27.0, 17.2 (d, J=25.2 Hz).

Unsaturated product B: ¹H NMR (300 MHz, CDCl₃): 9.06 (br s, 1H), 7.20 (d, J=8.1 Hz, 1H), 6.67 (br s, 1H), 5.69 (d, J=8.1 Hz, 1H), 5.58 (br s, 1H), 5.51 (br s, 1H), 5.29 (br s, 1H), 4.38-4.07 (m, 3H), 1.17 (s, 9H), 1.15 (s, 9H).

¹³C NMR (75 MHz, CDCl₃): 177.9, 177.8, 162.8, 150.6, 143.7, 140.2, 116.6, 103.5, 84.2, 80.0, 71.7, 63.1, 38.9, 38.7, 27.1, 27.0.

Starting material epimer C: ¹H NMR (300 MHz, CDCl₃): 9.55 (br s, 1H), 7.67 (d, J=8.2 Hz, 1H), 6.03 (s, 1H), 5.80 (d, J=8.2 Hz, 1H), 4.94 (d, J=7.7 Hz, 1H), 4.45-4.27 (m, 3H), 1.27 (s, 18H), 1.25 (s, 3H).

¹³C NMR (75 MHz, CDCl₃): 177.9, 177.5, 162.8, 150.8, 139.2, 102.7, 91.7, 78.5, 78.2, 73.3, 62.0, 39.0, 38.9, 27.2, 27.1, 21.0.

### Comparative Example 2: Fluorination of ((2R,3R,4R,5R)-3-(benzoyloxy)-5-(2,4-dioxo-3,4-dihydropyrimidin-1(2H)-yl)-4-hydroxy-4-methyltetrahydrofuran-2-yl)methyl benzoate with XTalFluor E

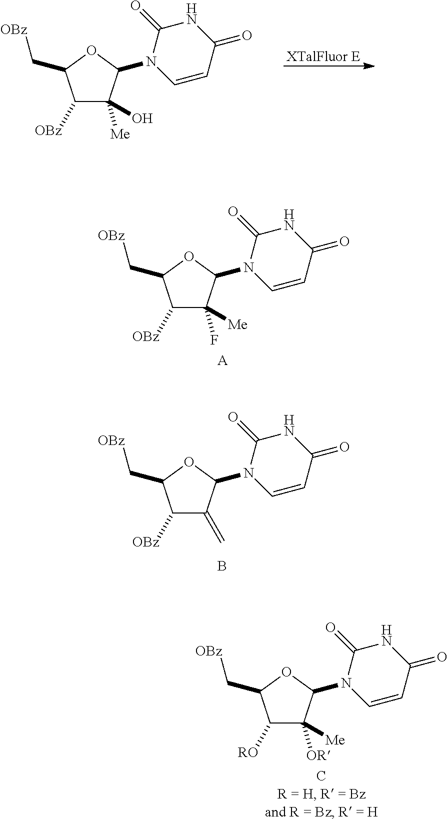

In a two-neck round bottom flask purged with nitrogen, triethylamine (30 microL, 0.21 mmol, 1 equiv) was dissolved in DCM (1.5 mL), and TEA.3HF (69 microL, 0.43 mmol, 2 equiv) was added. To this solution XTalFluor E (73 mg, 0.32 mmol, 1.5 equiv) was added at r.t, and the solution was cooled to −80° C. To this solution was added 3′,5′-di-O-benzoyl-2′-C-methyl arabino-uridine prepared according to Example 7 (61 mg, 0.14 mmol, 1 equiv) and the reaction mixture was stirred for 15 min, after which the cooling was removed. The reaction was stirred for 3 h at r.t. and 17 h at 40° C. HPLC control indicated a mixture of the starting material (57%), title compound A (12%), the unsaturated compound B (2%), and the starting material epimer C as a mixture of regio-isomers (29% total). Characterization data for product A:

¹H NMR (300 MHz, (CD₃)₂O) of A: 8.74 (br s, 1H), 8.09 (d, J=7.5 Hz, 2H), 8.02 (d, J=7.5 Hz, 2H), 7.70-7.41 (m, 7H), 6.28 (br d, J=18.6 Hz, 1H), 5.54 (dd, J=9.5 Hz, 21.9 Hz, 1H), 5.43 (d, J=8.1 Hz, 1H), 4.97-4.87 (m, 1H), 4.69-4.60 (m, 1H), 4.57-4.46 (m, 1H), 1.46 (d, J=22.3 Hz, 3H).

The yields of the fluorination processes of examples 8 to 11 and of comparative examples 1 and 2 are reported in Table 1.

TABLE 1

| Example | Protecting group | Deoxyfl. reagent | Yield |
|---|---|---|---|
| Comparative | Pivalate | XTalFluor E | 18% |
| Example 1 | C(O)C(CH₃)₃ | | |
| Comparative | Benzyl | XTalFluor E | 12% |
| Example 2 | | | |
| Example 8 | C(O)CF₃ | XTalFluor E | 42% (including |
| | | | deprotection step) |
| Example 9 | C(O)CH₂Cl | XTalFluor E | 47% |
| Example 10 | C(O)CCl₃ | XTalFluor E | 58% (no |
| | | | deprotection) |
| | | | 46% (including |
| | | | deprotection step) |
| Example 11 | Mesylate | XTalFluor E | 26% |

The processes carried out according to the present invention lead to a far higher yield with respect to the synthesis carried out with DAST and the nucleoside derivatives protected with protecting groups generically used in the prior art processes.

### Comparative Example 3

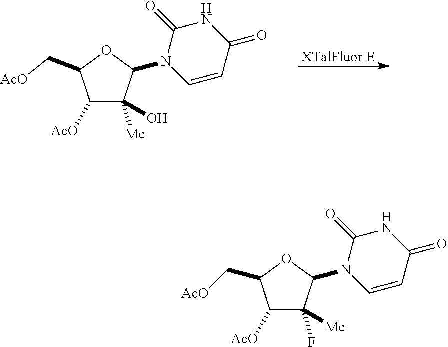 [SMILES: CC(=O)OC[C@H]1O[C@@H](n2ccc(=O)[nH]c2=O)[C@@](C)(O)[C@@H]1OC(C)=O ; "XTalFluor E" ; CC(=O)OC[C@H]1O[C@@H](n2ccc(=O)[nH]c2=O)[C@](C)(F)[C@@H]1OC(C)=O]

The acetyl-protected starting material was prepared according to the procedure described in: Nucleosides, Nucleotides and Nucleic Acids, 2011, 30, 886. The fluorination reaction was carried out according to the procedure described in comparative example 2 Yield: <26% (yield determined by NMR).

### Comparative Example 4

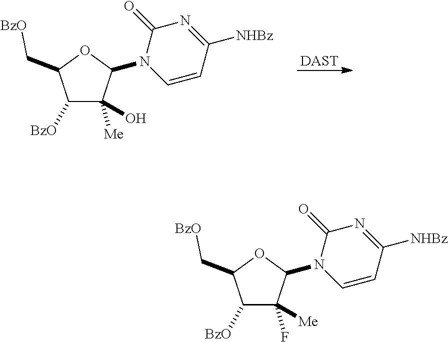

The substrate in this example is the Bz-protected cytidine-analogue. The fluorination reaction was described in: J. Med. Chem. 2005, 48, 5504, yield: 19% and WO 2005/003147, yield: 16%.

### Comparative Example 4a

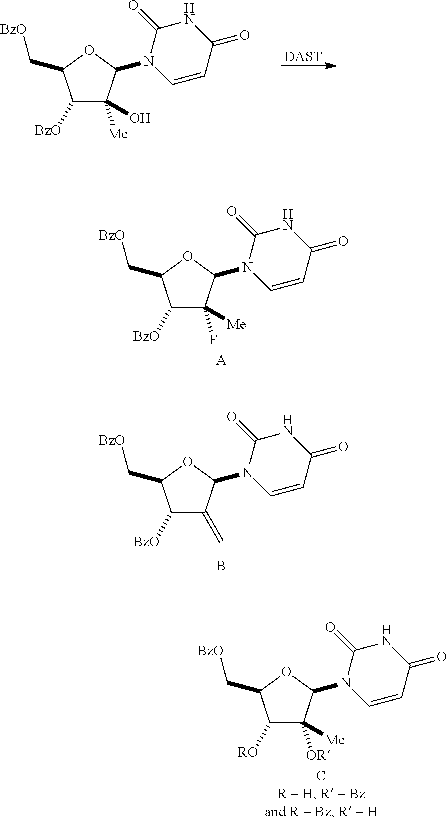

In a two-neck round bottom flask purged with nitrogen, 3′,5′-di-O-benzoyl-2′-C-methyl arabino-uridine prepared according to Example 7 (100 mg, 0.22 mmol, 1 equiv) was dissolved in DCM (2 mL) and the solution was cooled to −78° C. DAST (44 microL, 0.34 mmol, 1.5 equiv) was added and the cooling was removed. The reaction was stirred for 1 h after which all of the starting material was consumed. HPLC control indicated a mixture of the title compound A (20%), the unsaturated compound B (42%), and the starting material epimer C as a mixture of regio-isomers (38% total). Characterization data for product A:

¹H NMR (300 MHz, (CD₃)₂O) of A: 8.74 (br s, 1H), 8.09 (d, J=7.5 Hz, 2H), 8.02 (d, J=7.5 Hz, 2H), 7.70-7.41 (m, 7H), 6.28 (br d, J=18.6 Hz, 1H), 5.54 (dd, J=9.5 Hz, 21.9 Hz, 1H), 5.43 (d, J=8.1 Hz, 1H), 4.97-4.87 (m, 1H), 4.69-4.60 (m, 1H), 4.57-4.46 (m, 1H), 1.46 (d, J=22.3 Hz, 3H).

### Comparative Example 5

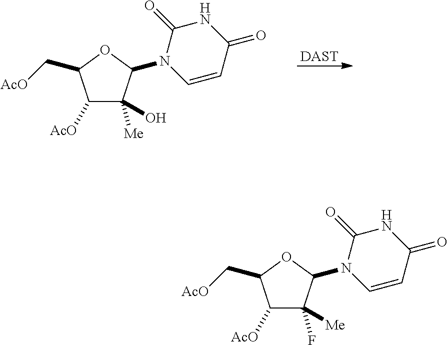

This reaction is described in: Nucleosides, Nucleotides and Nucleic Acids, 2011, 30, 886. Yield: 24%

### Comparative Example 6

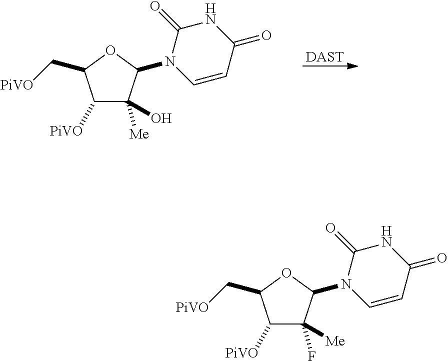

This reaction was carried out according to the procedure described in: J. Med. Chem. 2005, 48, 5504. Yield: <26% (yield determined by NMR).

### Comparative Example 7

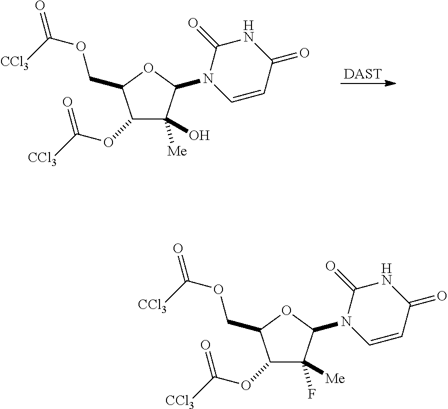

This reaction was carried out according to the procedure described in: J. Med. Chem. 2005, 48, 5504. Yield: <20% (yield determined by NMR).

The yield of example 10 was compared with the yields of the fluorination processes according to the prior art carried out with DAST as the fluorinating agent and the protecting groups commonly used in the prior art benzyl (Bz), acetyl (Ac) and pivaloyl (Piv) of comparative examples 4 to 6, with the yields of the comparative examples 1 to 3 wherein the very same protecting groups were used and XtalFluor E was used as fluorinating agent and with the yield of comparative example 7 wherein the (CCl₃CO) protecting group was used in combination with the prior art fluorinating agent DAST. The yields are reported in below Table 2.

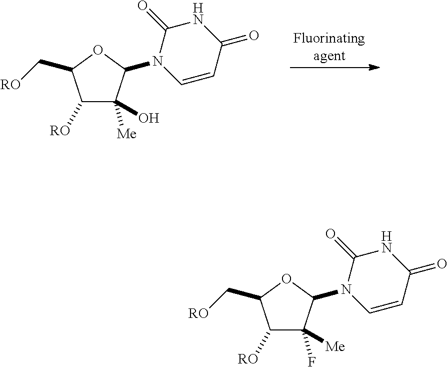

TABLE 2

| | | | | Protecting |
|---|---|---|---|---|
| | | | | group according |
| Fluorinating | Prior Art Protecting groups R | | | to the invention |
| agent | Bz | Ac | Piv | (CCl₃CO) |
| DAST | Comparative | Comparative | Comparative | Comparative |
| | example 4 | example 5 | example 6 | example 7 |
| | Nucleobase = | Nucleobase = | Nucleobase = | Nucleobase = |
| | uridine: <20% | uridine: 24% | uridine: <26% | uridine: <20% |
| | Comparative | | | |
| | example 4 | | | |
| | (Nucleobase = | | | |
| | N-Bz-Cytidine) | | | |
| | 16-19% | | | |
| (XtalFluor E) | Comparative | Comparative | Comparative | Example 10 |
| | example 2 | example | example 1 | 58% (without |
| | 12% | 3 <26% | 18% | deprotection) |

The yield of 58% of the process of example 10 is far higher than the yield obtained with the fluorination processes of comparative examples 1 to 3 and of comparative examples 4 to 7, showing that the combination of the fluorinating agent of the invention and the protecting groups of the invention lead to an improved process.

### CITED PRIOR ART

WO 2005/003147 A
WO 2006031725 A
WO 2008/045419 A
J. Org. Chem 2009, 74, pp. 6819
WO 2008/090046 A
WO 2007/075876 A
J. Am. Chem. Soc, 2014, 136, pp. 5900
WO 2013/096680 A
Nucleosides, Nucleotides and Nucleic Acids, 2012, 31, pp. 277
Carbohydr. Chem. 2006, 25, pp. 461
J. Med. Chem. 2005, 48, pp. 5504
Nucleosides, Nucleotides and Nucleic Acids, 2011, 30, pp. 886
Tetrahedron Lett. 2007, 48, pp. 4441
Chemical and Pharmaceutical Bulletin, 1988, 36, pp. 945
J. Org. Chem. 2003, 68, pp. 6799
WO 2008/121634 A

1. A process comprising
(i) providing a mixture comprising a compound of formula (I) or isomers, stereoisomers, diastereomers, enantiomers, or salts thereof 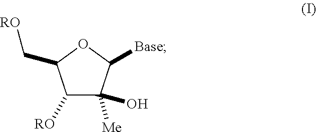
(ii) subjecting the mixture provided in (i) to fluorinating conditions in the presence of a fluorination agent selected from the group consisting of diethylamino(difluoro)sulfonium tetrafluoroborate and difluoro(morpholino) sulfonium tetrafluoroborate obtaining a mixture comprising a compound of formula (II) 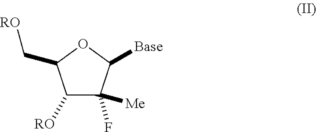
or isomers, stereoisomers, diastereomers, enantiomers or salts thereof,
(iii) optionally subjecting the mixture obtained in (ii) to deprotection conditions, obtaining a mixture comprising the compound of formula (III) or isomers, stereoisomers diastereomers, enantiomersor salts thereof 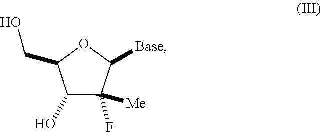
wherein at each occurrence,
R is an inert electron withdrawing OH protecting group; and
Base is a purinyl residue or a pyrimidinyl residue linked to the furanose ring according to formulae (I), (II) and (III) through a carbon or a nitrogen atom.

2. The process of claim 1, wherein
R is selected from the group consisting of X₃₋ₙHₙCC(O) wherein X is halogen and n is 0, 1, or 2; or
R is selected from the group consisting of SO₂Me, SO₂-p-Me-Ph (tosyl), SO₂-p-NO₂-Ph (para-nosyl), SO₂-o-NO₂-Ph (ortho-nosyl) and SO₂CF₃ (triflyl).

3. The process of claim 1, wherein
R is selected from SO₂Ph or SO₂-o-CF₃-Ph (ortho-trifluoromethylphenyl); or
R is 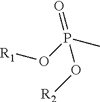
wherein R₁ and R₂ are independently selected from alkyl, aryl or R₁ and R₂ taken together are a (CH₂)_{q} group that forms a ring with the oxygen atoms to which R₁ and R₂ are bound and wherein q is 2, 3, 4, 5, 6, 7; or
R is selected from CH═CH₂—CO₂R₃ or C(O)—CH₂—CO₂R₃ wherein R₃ is selected from the group consisting of alkyl, aryl and cycloalkyl; or
wherein the radical R attached to the oxygen in position 5′ of the sugar moiety taken together with the radical R attached to the oxygen in position 3′ of the sugar moiety forms a group selected from C(O), C(O)—(CH₂)ₜ—CO or 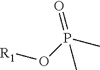
wherein R₄ is selected from the group consisting of alkyl, aryl and cycloalkyl and wherein t is 1 or 2.

4. The process of claim 1, wherein Base is selected from the group consisting of uridine, protected uridine, thymine, protected thymine, cytidine, protected cytidine, adenosine, protected adenosine, guanine and protected guanine.

5. The process of claim 1, wherein the compound of formula (III) is the compound of formula (III)

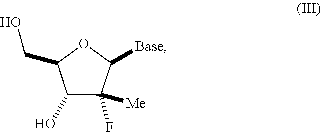

6. The process of claim 1, wherein the mixture provided in (i) further comprises one or more organic solvents, wherein the mixture provided in (i) further comprises one or more organic bases.

7. The process of claim 6, wherein in the mixture provided in (i), the one or more bases and the compound of formula (I) are present in a molar ratio of the one or more bases relative to the compound of formula (I) in the range of from 0.1:1 to 3:1, wherein, if more than one base is comprised in the mixture, the molar ratios relate to the total molar amount of all bases.

8. The process of claim 1, wherein the mixture provided in (i) further comprises an agent selected from the group consisting of triethylamine trihydrofluoride (TEA 3HF), triethylamine dihydrofluoride (TEA 2HF), diazabicycloundec-7-ene (DBU), and a mixture of two or more thereof.

9. The process of claim 1, wherein prior to (ii), the diethylamino(difluoro)sulfonium tetrafluoroborate or the difluoro(morpholino) sulfonium tetrafluoroborate is present in a molar ratio of the diethylamino(difluoro) sulfonium tetrafluoroborate or the difluoro(morpholino)sulfonium tetrafluoroborate relative to the compound of formula (I) in the range of from 0.1:1 to 3:1.

10. The process of claim 1, wherein the temperature during (ii) is in the range of from −80 to 40° C.

11. The process of claim 1, wherein prior to (iii), the process further comprises
(ii′) separating the compound of formula (II) from the mixture obtained in (ii).

12. The process of claim 1, comprising (iii), wherein (iii) further comprises adding to the mixture obtained in (ii) one or more deprotection reagents selected from the group consisting of water, a mixture of NH₃ and MeOH, and a mixture of NaOMe and MeOH.

13. The process of claim 1, which further comprises
(iv) separating the compound of formula (III) from the mixture obtained in step (iii).

14. The process of claim 1, further comprising providing the mixture according to (i) by a process comprising
(a) providing a mixture comprising a compound of formula (IV) 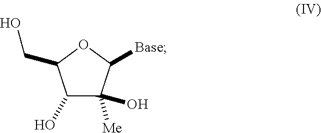
(b) subjecting the mixture provided in (a) to protection conditions in the presence of an OH-protecting agent comprising an inert electron-withdrawing OH-protecting group R, obtaining a mixture comprising the compound of formula (I)

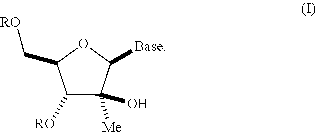

15. The process of claim 14, wherein the mixture provided in (a) comprises one or more organic solvents.

16. The process of claim 14, wherein the mixture provided in (a) further comprises one or more organic or one or more inorganic bases or mixtures of two or more thereof.

17. The process of claim 14, wherein the mixture provided in (a) further comprises a reagent selected from the group consisting of N,N-dialkylaminopyridines and pyridine, wherein the reagent selected from the group consisting of N,N-dialkylaminopyridines and pyridine and the compound of formula (IV) are present in a molar ratio of reagent selected from the group consisting of N,N-dialkylaminopyridines and pyridine relative to the compound of formula (IV) in the range of from 0.1:1 to 0.6:1, wherein the process optionally further comprises (c) separating the compound of formula (I) from the mixture obtained in (b).

18. A compound of formula (I) or a compound of formula (II) 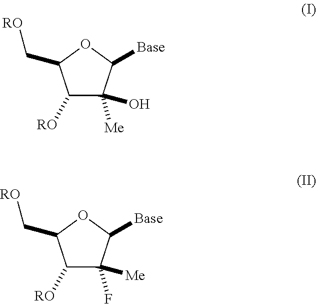
wherein at each occurrence,
R is an inert electron withdrawing OH protecting group or R is 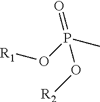
wherein R₁ and R₂ are independently selected from alkyl, aryl or R₁ and R₂ taken together are a (CH₂)ₙ group that forms a ring with the oxygen atoms to which R₁ and R₂ are bound and wherein q is 2, 3, 4, 5, 6, 7; or R is selected from CH═CH₂—CO₂R₃ or C(O)—CH₂—CO₂R₃ wherein R₃ is selected from the group consisting of alkyl, aryl and cycloalkyl; or wherein the radical R attached to the oxygen in position 5′ of the sugar moiety taken together with the radical R attached to the oxygen in position 3′ of the sugar moiety forms a group selected from C(O), C(O)—(CH₂)ₜ—CO or 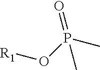
wherein R₄ is selected from the group consisting of alkyl, aryl and cycloalkyl and wherein t is 1 or 2; and
Base is a purinyl residue or a pyrimidinyl residue linked to the furanose ring according to formulae (I) and (II)) through a carbon or a nitrogen atom.

19. (canceled)

20. A mixture comprising a compound of formula (I) or a compound of formula (II) according to claim 18.

21. (canceled)

22. A mixture comprising a compound of formula (II) or a compound of formula (III), obtainable or obtained by a process according to claim 1, the mixture having a content, based on the weight of the mixture, of at most 1000 weight-ppm of one or more compounds of formula (I′) or one or more compounds of formula (IV′) or one or more compounds of formula (V′) or one or more compounds of formula (VI′) or mixtures of two or more thereof 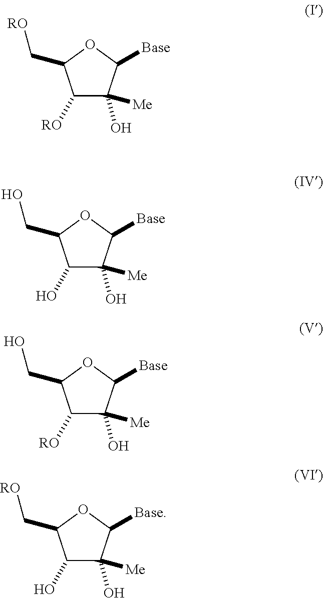
wherein at each occurrence,
R is an inert electron withdrawing OH protecting group; and
Base is a purinyl residue or a pyrimidinyl residue linked to the furanose ring according to formulae (II), (I′), (IV′), (V′) and (VI′) through a carbon or a nitrogen atom,
wherein, in case the mixture comprises more than one compound of formula (I′) or of formula (IV′) or of formula (V′) or of formula (VI′) said weight-ppm values relate to each individual compound of formula (I′) or of formula (IV′) or of formula (V′) or of formula (VI′).
